SEQUENCE LISTING (357 sequences)

SEQ ID NO: 1 - DNA, 127 bp, Homo sapiens
aacaaatcttcatcttggaatagcctgtgagaatgcctaatcatctacgaatgttactttggcaccatctactggacagattaaataacaaccaactcactgtggattagacctacttctatttcag

SEQ ID NO: 2 - DNA, 20 bp, Homo sapiens
atagcctgtgagaatgccta

SEQ ID NO: 3 - DNA, 20 bp, Homo sapiens
atccacagtgagttggttgt

SEQ ID NO: 4 - DNA, 84 bp, Homo sapiens
ttcctggaaaacaaaagtatttctttcatagcccagctagcatgataaatcagcgagtcagaattctagctttgttgtaaggtt

SEQ ID NO: 5 - DNA, 20 bp, Homo sapiens
tcctggaaaacaaaagtatt

SEQ ID NO: 6 - DNA, 21 bp, Homo sapiens
aaccttacaacaaagctagaa

SEQ ID NO: 7 - DNA, 71 bp, Homo sapiens
cactaagccttggggatccagctgcttaaggactaagaccgtatctagctccttttagtatttccacagca

SEQ ID NO: 8 - DNA, 20 bp, Homo sapiens
actaagccttggggatccag

SEQ ID NO: 9 - DNA, 21 bp, Homo sapiens
tgctgtggaaatactaaaagg

SEQ ID NO: 10 - DNA, 155 bp, Homo sapiens
tcctccagaggtaatcctgtgatcagcactaacaccacataccagccctttcatcagcttgttggagaagcatctttacttcccgccaagcagtgacctagataccatctcacaccagttagaatcaggatcattaaaaagtcaagaaaaaacag

SEQ ID NO: 11 - DNA, 20 bp, Homo sapiens
ctccagaggtaatcctgtga

SEQ ID NO: 12 - DNA, 21 bp, Homo sapiens
tggtgtgagatggtatctagg

SEQ ID NO: 13 - DNA, 146 bp, Homo sapiens
tccaagtataatccatgaatcttgtttaaatatagatcaaataaaccactataccaaaaacatcaaaagacaactgggtaaattttttaaatgactagctatttgatgttaaggaagtaatgttactctcttatatacaatttgaa

SEQ ID NO: 14 - DNA, 22 bp, Homo sapiens
gtataatccatgaatcttgttt

SEQ ID NO: 15 - DNA, 22 bp, Homo sapiens
ttcaaattgtatataagagagt

SEQ ID NO: 16 - DNA, 132 bp, Homo sapiens
atggaaccgaaacttcaagtagtttcatacgtatcacattgacagttttctctaagttttctggtcttatgactcgttgtttcattattaaaactgtgccagtgtatgcatagggcttagaaattttttaat

SEQ ID NO: 17 - DNA, 18 bp, Homo sapiens
atggaaccgaaacttcaa

SEQ ID NO: 18 - DNA, 22 bp, Homo sapiens
ttaataatgaaacaacgagtca

SEQ ID NO: 19 - DNA, 157 bp, Homo sapiens
acaggatccttcctgaagacaccaccttggggagggtgaaggataaagaatttgatcagaaatcaagggtggtgagatacatgttaaggatgaataaactggccttttaggattcttgctaaaattagacaatgcagaggcaaccacagagtccaag

SEQ ID NO: 20 - DNA, 19 bp, Homo sapiens
ttcctgaagacaccacctt

SEQ ID NO: 21 - DNA, 18 bp, Homo sapiens
cttggactctgtggttgc

SEQ ID NO: 22 - DNA, 173 bp, Homo sapiens
aatttccattaaatcttgttcgttgctttactgaggcactgaagttaccaatgttccactggttgacctgcggggctatctctaggttatgttactccagaaaatgaattgtgtataaaagaggccttggaggaaggcgttttattcacatcagttgttttgcacattgctta

SEQ ID NO: 23 - DNA, 20 bp, Homo sapiens
actgaggcactgaagttacc

SEQ ID NO: 24 - DNA, 20 bp, Homo sapiens
taagcaatgtgcaaaacaac

SEQ ID NO: 25 - DNA, 191 bp, Homo sapiens
tcggtttcagcaggaaagttatttttaataacttccctgtatttcttggtttcagttattaattaactcattaatgctaaactttgtgatcctaggttaaaaaacatattcaagatagcttcagaatgtttggtatacaagtaggtctggctaaatataagtgttagctttctcaagcatctaaatgctgg

SEQ ID NO: 26 - DNA, 20 bp, Homo sapiens
gcaggaaagttatttttaat

SEQ ID NO: 27 - DNA, 21 bp, Homo sapiens
tgcttgagaaagctaacactt

SEQ ID NO: 28 - DNA, 166 bp, Homo sapiens
aagttatttttaataacttccctgtatttcttggtttcagttattaattaactcattaatgctaaactttgtgatcctaggttaaaaaacatattcaagatagcttcagaatgtttggtatacaagtaggtctggctaaatataagtgttagctttctcaagcatc

SEQ ID NO: 29 - DNA, 20 bp, Homo sapiens
atttttaataacttccctgt

SEQ ID NO: 30 - DNA, 20 bp, Homo sapiens
cacttatatttagccagacc

SEQ ID NO: 31 - DNA, 152 bp, Homo sapiens
attcattgtgtagaaagtgcctgactcagtgtttggaaattgtctgacttttcctcatatatagtgtggtttcatgttattgtatataagaactgacatgaactctgtttacaataatctcccagtgccataaagaccataataaataatat

SEQ ID NO: 32 - DNA, 20 bp, Homo sapiens
cagtgtttggaaattgtctg

SEQ ID NO: 33 - DNA, 20 bp, Homo sapiens
ggcactgggagattattgta

SEQ ID NO: 34 - DNA, 163 bp, Homo sapiens
cactgggtcctgttgttaagtacacataataccacacaggagaaaatcaggctaattgtaaatgggcaacctacttaattgtttcattaaaaagcatacagattacatttacactatagctagtcttgtttgtttttttattttgcaaaagtaattacggccc

SEQ ID NO: 35 - DNA, 20 bp, Homo sapiens
tcctgttgttaagtacacat

SEQ ID NO: 36 - DNA, 18 bp, Homo sapiens
gggccgtaattacttttg

SEQ ID NO: 37 - DNA, 108 bp, Homo sapiens
ctactcagtaggcactttgtgtctagaaacttctgtgtcaacggttttccctctctctggaattcatcaggacagaagtgattggtgtggtggaagagggttgtgsta

SEQ ID NO: 38 - DNA, 21 bp, Homo sapiens
actcagtaggcactttgtgtc

SEQ ID NO: 39 - DNA, 18 bp, Homo sapiens
tcttccaccacaccaatc

SEQ ID NO: 40 - DNA, 144 bp, Homo sapiens
tggcttttcaaaggtaaaatttactaagtgtattaatattttaccaatttccagccaggagagtatgaatgttgcattattacattgctttgaaacaaagcattagtcttaattcagaagtttaaattcagatgttaacgttgc

SEQ ID NO: 41 - DNA, 19 bp, Homo sapiens
tggcttttcaaaggtaaaa

SEQ ID NO: 42 - DNA, 21 bp, Homo sapiens
gcaacgttaacatctgaattt

SEQ ID NO: 43 - DNA, 110 bp, Homo sapiens
taagtattgaagaaaggagaatttaaattacttcatatacctgataaaggaaaacatatacaaggcaaataaacatcttagatcatgacatataaaataatagattatta

SEQ ID NO: 44 - DNA, 20 bp, Homo sapiens
ttgaagaaaggagaatttaa

SEQ ID NO: 45 - DNA, 22 bp, Homo sapiens
attttatatgtcatgatctaag

SEQ ID NO: 46 - DNA, 165 bp, Homo sapiens
tgcagagattacaggtgtgagccaccgtgcccagcctcataaccgtttcaactactttttcacttgacaagcagatgtgaagttaacaaagtcacccatatttgaaataaagatagtatattcctggggtaggcagaggcagttgaggatcatgaaataactatg

SEQ ID NO: 47 - DNA, 19 bp, Homo sapiens
agagattacaggtgtgagc

SEQ ID NO: 48 - DNA, 19 bp, Homo sapiens
atgatcctcaactgcctct

SEQ ID NO: 49 - DNA, 178 bp, Homo sapiens
tgcaatgaaactcaaaagagaaaagttaacaggtgcaaaaggtagttttattataaaaggagggtaggcaacaagaatatgtttaatttttcttccttttcatgagtaaggacaagagtgtcatatatgtgaatatttttatttaattttaagtagaaatctgtttttaaaatatggg

SEQ ID NO: 50 - DNA, 20 bp, Homo sapiens
tgaaactcaaaagagaaaag

SEQ ID NO: 51 - DNA, 20 bp, Homo sapiens
acagatttctacttaaaatt

SEQ ID NO: 52 - DNA, 102 bp, Homo sapiens
ccaccattcatcaaaactttgatactggactcaattgtgaatttgacttgaaatttgataatgcttttgttttactgttctgctcagcaaaatagtacatgt

SEQ ID NO: 53 - DNA, 20 bp, Homo sapiens
caaaactttgatactggact

SEQ ID NO: 54 - DNA, 19 bp, Homo sapiens
acatgtactattttgctga

SEQ ID NO: 55 - DNA, 166 bp, Homo sapiens
gcctgcataaagtgaggatggtgtagtaattgggtatctccagttataaacacaaaaagcatgatagagctgggactgtgattgcaggaaagcaatagtcactccaaaaggagatcctcatgatatgaatacggaagaaacaatatttcctgctaatgtagtagcc

SEQ ID NO: 56 - DNA, 20 bp, Homo sapiens
cctgcataaagtgaggatgg

SEQ ID NO: 57 - DNA, 21 bp, Homo sapiens
tgaggatctccttttggagtg

SEQ ID NO: 58 - DNA, 154 bp, Homo sapiens
gcaaaggggtactctatgtaatgaacatgacctggcagtactgacatctcctgagggactgttagaagtgcagactcttgtatcttttctcaagtctatgaaatctagacttcattttaacaagatgacccgatatttacatacacattaaagt

SEQ ID NO: 59 - DNA, 20 bp, Homo sapiens
gcaaaggggtactctatgta

SEQ ID NO: 60 - DNA, 20 bp, Homo sapiens
tatcgggtcatcttgttaaa

SEQ ID NO: 61 - DNA, 174 bp, Homo sapiens
gtatctaacaaagctctgtccaaaattttgaatttctcgttaaaagcatcatgattatagaacagaggttacaatcaattattcagtcacacaatcactctcatcagtcattaaggtgcgtacctggtgttccagttattcagtgtggtataacaaactacctggaacttaatg

SEQ ID NO: 62 - DNA, 22 bp, Homo sapiens
tctaacaaagctctgtccaaaa

SEQ ID NO: 63 - DNA, 21 bp, Homo sapiens
ccacactgaataactggaaca

SEQ ID NO: 64 - DNA, 202 bp, Homo sapiens
agagtggttaagtgacttgatcaattcctcaggtggggattcaagctcttaaagctgtagactatgtcgtccaaacaaacactgacatgaatatgacttccaataggcaagaaaagaggcctaggtcgagatactgcaagacatgcaagcaatctagtaatggcataaaacctgctatccgaattggctaaaattatgtatt

SEQ ID NO: 65 - DNA, 20 bp, Homo sapiens
ggtggggattcaagctctta

SEQ ID NO: 66 - DNA, 22 bp, Homo sapiens
ggatagcaggttttatgccatt

SEQ ID NO: 67 - DNA, 156 bp, Homo sapiens
ttctttctcacacaatgggttccattcccactactactccattcaaattgaagtgccttcaatgattattaaaaaactctctttaaaatagctcacgtaaccttacatcctttgactgaggctcaactcatgtcaatgcttcagtatcaacttttc

SEQ ID NO: 68 - DNA, 21 bp, Homo sapiens
aatgggttccattcccactac

SEQ ID NO: 69 - DNA, 20 bp, Homo sapiens
tgagcctcagtcaaaggatg

SEQ ID NO: 70 - DNA, 202 bp, Homo sapiens
atttgtaataacatttagtaagtatttatttgaggagtttgaattttgttcttgtttatcttgttctctttcttcgtagattagttggtgttaacatcaataggataaccctttctttcagcatatgtgaatgaaataaaccaattattgccactttccaggttaaccagaatatacatagatacgaggacagtggactgtt

SEQ ID NO: 71 - DNA, 22 bp, Homo sapiens
ttgaggagtttgaattttgttc

SEQ ID NO: 72 - DNA, 20 bp, Homo sapiens
aacctggaaagtggcaataa

SEQ ID NO: 73 - DNA, 171 bp, Homo sapiens
tagggcagagagagcaagcaagctctctaccttctcatataagggcactaatcccaccatgaaggcgccactgtcatgacctgattatgtcacaaagaccccggggcaaatattaccactgtgaggagtacagttttagcatgtgaattttggaagaacacaaacatttag

SEQ ID NO: 74 - DNA, 21 bp, Homo sapiens
gcaagcaagctctctaccttc

SEQ ID NO: 75 - DNA, 22 bp, Homo sapiens
tgttcttccaaaattcacatgc

SEQ ID NO: 76 - DNA, 195 bp, Homo sapiens
attctaattttaaatatcattgatgtagaacattctatttcactattccttcattttattattatgggaaattatatacagttctccagatttttaaagccttgctaacatgttttaagtcacacaaatattctcctgtgggaaaatgacagtaatttagtgtgcaacaattatatagaactatttttcaaactt

SEQ ID NO: 77 - DNA, 21 bp, Homo sapiens
atttcactattccttcatttt

SEQ ID NO: 78 - DNA, 22 bp, Homo sapiens
taattgttgcacactaaattac

SEQ ID NO: 79 - DNA, 138 bp, Homo sapiens
actgtcatggacttaaacaattgtctttgaattgtcttttttcatacttttatttgcatctttccactaaaaagatggcacaaagtaatcctagtttacattttttaccatgtaattccatattactttttcctgaaa

SEQ ID NO: 80 - DNA, 20 bp, Homo sapiens
actgtcatggacttaaacaa

SEQ ID NO: 81 - DNA, 22 bp, Homo sapiens
ttcaggaaaaagtaatatggaa

SEQ ID NO: 82 - DNA, 119 bp, Homo sapiens
aaagaaaaaaaagccacagaaatcagtcctagagaaaaccgatctatgagctgcctgaaaataattataaaataactatcataaaaatgcccagtgagatataagaaaacacagacaac

SEQ ID NO: 83 - DNA, 21 bp, Homo sapiens
aaaaagccacagaaatcagtc

SEQ ID NO: 84 - DNA, 22 bp, Homo sapiens
ttcttatatctcactgggcatt

SEQ ID NO: 85 - DNA, 143 bp, Homo sapiens
caaggtcagagaagttatcttggatggtagaagagaagaaaggagaagaaaggataagcagaaaatcaaaaagggcataaaaaaattactggggaaaataattcttagtcactcaccatttcttatgtttgtgaaaacagaaa

SEQ ID NO: 86 - DNA, 22 bp, Homo sapiens
ggatggtagaagagaagaaagg

SEQ ID NO: 87 - DNA, 22 bp, Homo sapiens
tcacaaacataagaaatggtga

SEQ ID NO: 88 - DNA, 136 bp, Homo sapiens
gaccacaattcacaaatgcaaagatgcagaaccaacctaagtggccactgactaatgagaggataaagaagatgtggcatatatatatcagggactactactcagccattacaaggaacaaaataatgtcttttgc

SEQ ID NO: 89 - DNA, 20 bp, Homo sapiens
tgcaaagatgcagaaccaac

SEQ ID NO: 90 - DNA, 22 bp, Homo sapiens
ttttgttccttgtaatggctga

SEQ ID NO: 91 - DNA, 160 bp, Homo sapiens
gaccacaattcacaaatgcaaagatgcagaaccaacctaagtggccactgactaatgagaggataaagaagatgtggcatatatacatcagggactacttctcagccattacaaggaacaaaataatgtcttttgcaacaacttggatagagctggaggc

SEQ ID NO: 92 - DNA, 20 bp, Homo sapiens
tgcaaagatgcagaaccaac

SEQ ID NO: 93 - DNA, 21 bp, Homo sapiens
gcctccagctctatccaagtt

SEQ ID NO: 94 - DNA, 160 bp, Homo sapiens
aatcctagaccttggattgcaagagactccttaatatcttcccatgtccacatttccttcacatagtttgaatgtggcttctattatatacagatacaagattcaaatccaacctctatgatgactggtcttgtgaataagcagaagaggcactaacaat

SEQ ID NO: 95 - DNA, 22 bp, Homo sapiens
ccttaatatcttcccatgtcca

SEQ ID NO: 96 - DNA, 22 bp, Homo sapiens
attgttagtgcctcttctgctt

SEQ ID NO: 97 - DNA, 162 bp, Homo sapiens
aagagaagtgaggtcagcagctgcaagccacctccgtcatttagaaaagcttcatgatgtagtgtgtcgtttcgatgtgacactgtctcacagagttaaaatgatgttaaggaactgttcaatggaaatttagaaatttctctttttctcaattttagtgta

SEQ ID NO: 98 - DNA, 20 bp, Homo sapiens
gagaagtgaggtcagcagct

SEQ ID NO: 99 - DNA, 22 bp, Homo sapiens
tttctaaatttccattgaacag

SEQ ID NO: 100 - DNA, 160 bp, Homo sapiens
atggctgaatagtattcccttgtgtatatatctatttatccttttattcattgatggacacttaggctgattttctctcttctcatggctggcttctcatcaccctttggtcctcctgtatcctcgtgtaataaagctcttccccaatatctcgatagat

SEQ ID NO: 101 - DNA, 22 bp, Homo sapiens
ggctgaatagtattcccttgtg

SEQ ID NO: 102 - DNA, 20 bp, Homo sapiens
tcgagatattggggaagagc

SEQ ID NO: 103 - DNA, 199 bp, Homo sapiens
cattttaacttgattacctccacaaagactattccagaataaggttatgttctgaggtattaggggttacaacttcaacatatgaattttgagtggacacaattcaacccatagcacctccgtgtaagagctgggaagggaaagtggctaagttgtgcaaatgtgcacattggttggagatgattaacttctggcatgt

SEQ ID NO: 104 - DNA, 22 bp, Homo sapiens
cctccacaaagactattccaga

SEQ ID NO: 105 - DNA, 20 bp, Homo sapiens
cactttcccttcccagctct

SEQ ID NO: 106 - DNA, 157 bp, Homo sapiens
agggggaaattggcaatctgattctaaaattcatacggaaaaaaacaatggagttagaataactaaaacaagtccgaaaaagaaaaagaaatggaggactaatgctacctgatttcaagtcttatcttataaatctacatcaataaaggacaagttg

SEQ ID NO: 107 - DNA, 20 bp, Homo sapiens
gaaattggcaatctgattct

SEQ ID NO: 108 - DNA, 21 bp, Homo sapiens
caacttgtcctttattgatgt

SEQ ID NO: 109 - DNA, 173 bp, Homo sapiens
tctgtgtttgtctatgttgataaaacattgaaatgccaaatagctcaaaggtcattcacttaagaaatctaagtactgataacatcttagccccgattcttcataggcattgttaagcctattataattttggttcagagagaaggtaaactatattccagacaggcatataa

SEQ ID NO: 110 - DNA, 22 bp, Homo sapiens
ctatgttgataaaacattgaaa

SEQ ID NO: 111 - DNA, 20 bp, Homo sapiens
gcctgtctggaatatagttt

SEQ ID NO: 112 - DNA, 126 bp, Homo sapiens
tgcagggcatataatctaagctgtaaacgtcctgtcagaagacaacatattcatcttgctaaggtttaagctatatgactggcactgtgctcaactcagagtcattgaatgaacagtatttattta

SEQ ID NO: 113 - DNA, 22 bp, Homo sapiens
cagggcatataatctaagctgt

SEQ ID NO: 114 - DNA, 21 bp, Homo sapiens
caatgactctgagttgagcac

SEQ ID NO: 115 - DNA, 132 bp, Homo sapiens
ttcacattattcccttaaaataaactctctccctcccctctcccgtctcaaccttgtccctttctttatataatgggtaattcgttaatgtcagcagaatagttttggggccataatggcaagtatcacgtg

SEQ ID NO: 116 - DNA, 19 bp, Homo sapiens
aactctctccctcccctct

SEQ ID NO: 117 - DNA, 20 bp, Homo sapiens
tatggccccaaaactattct

SEQ ID NO: 118 - DNA, 110 bp, Homo sapiens
tcaggaagcaacaagtactgggcagattgatactgtagctaggctctagctctatacctctagaataaatgttacaaactagcaacttgaaagctaaacctggcccacag

SEQ ID NO: 119 - DNA, 20 bp, Homo sapiens
acaagtactgggcagattga

SEQ ID NO: 120 - DNA, 20 bp, Homo sapiens
gccaggtttagctttcaagt

SEQ ID NO: 121 - DNA, 106 bp, Homo sapiens
tggttcttgagaattttatatcaggagaaacactgtcagtctgtattgaaaggaacagagaaaattcgaaattaaagaagactattaaacctccaaaattctggca

SEQ ID NO: 122 - DNA, 22 bp, Homo sapiens
ttttatatcaggagaaacactg

SEQ ID NO: 123 - DNA, 21 bp, Homo sapiens
ccagaattttggaggtttaat

SEQ ID NO: 124 - DNA, 171 bp, Homo sapiens
gcatcaaactacacactgtcattcctcctttatctccaaaagcttgaaaattcctcacttgtatctcattctttctctcttagaaaactgatcacctctgatgaattagaacggaatgaccaagctttgggagaggcaaaagaatctcggtgttaaagactcagagtttaa

SEQ ID NO: 125 - DNA, 22 bp, Homo sapiens
tgtcattcctcctttatctcca

SEQ ID NO: 126 - DNA, 20 bp, Homo sapiens
ttcttttgcctctcccaaag

SEQ ID NO: 127 - DNA, 163 bp, Homo sapiens
ttgaaaattaagaaaccctggcacagtgttgactggagccacttaccttaatagaaaataaagctcacatatatccataatgaaaagcagagaccagcacaaccatagtcacctgacagttttaaaatccaaggccaggatcttctcaactcaggcccactca

SEQ ID NO: 128 - DNA, 20 bp, Homo sapiens
accctggcacagtgttgact

SEQ ID NO: 129 - DNA, 20 bp, Homo sapiens
tgggcctgagttgagaagat

SEQ ID NO: 130 - DNA, 20 bp, Homo sapiens
tttttcccatttccaactct

SEQ ID NO: 131 - DNA, 122 bp, Homo sapiens
aaaaaaaaagatgagacaggcaggtgcgaaagaaataaaagtcaaaactgatccagttgggaaactcagaattgacagttacgtgtcctttcatttattgatattttgagattcacaggggt

SEQ ID NO: 132 - DNA, 20 bp, Homo sapiens
aaaagatgagacaggcaggt

SEQ ID NO: 133 - DNA, 20 bp, Homo sapiens
acccctgtgaatctcaaaat

SEQ ID NO: 134 - DNA, 150 bp, Homo sapiens
gagttaaataaagcacttgcttctattgtttgtacctaaacttaacagaacacagtaagtaacaagtcattgggatgcagaaaagaaaaaagagagtgaaggaaggagaaaaggtgaagggagaatggaagagaggaagggagggaggaa

SEQ ID NO: 135 - DNA, 21 bp, Homo sapiens
gcacttgcttctattgtttgt

SEQ ID NO: 136 - DNA, 20 bp, Homo sapiens
cccttcctctcttccattct

SEQ ID NO: 137 - DNA, 129 bp, Homo sapiens
aaacgagccaccagtgggagcactgcaggtatctgtgtgagacccgtacttcacaactcctgctttccctccataaagtagcttgcattttccacattgactttgcagttctttggtatctgtattggt

SEQ ID NO: 138 - DNA, 18 bp, Homo sapiens
gtgggagcactgcaggta

SEQ ID NO: 139 - DNA, 21 bp, Homo sapiens
acagataccaaagaactgcaa

SEQ ID NO: 140 - DNA, 145 bp, Homo sapiens
tggacacctttcaacttagaaatcataaacagattcatttccttaaagttaatgaaaagaattaacagaccctcctcaaaaaagacatatatgcagcctacaatcatatgaaaaaaagttcaacattactgttcagcaaatcaaa

SEQ ID NO: 141 - DNA, 20 bp, Homo sapiens
tggacacctttcaacttaga

SEQ ID NO: 142 - DNA, 22 bp, Homo sapiens
gaacagtaatgttgaacttttt

SEQ ID NO: 143 - DNA, 155 bp, Homo sapiens
tggatacattcctagaaatagatggaaactgctcttgcaaaaagcttagcacatgttaaaaattttagaaacaatttgccaaagtttatttagtctagtgattttgacaggttaaatggaccctttgagatcttttttcctcaagtacaaaggct

SEQ ID NO: 144 - DNA, 21 bp, Homo sapiens
tcttgcaaaaagcttagcaca

SEQ ID NO: 145 - DNA, 21 bp, Homo sapiens
aaaaagatctcaaagggtcca

SEQ ID NO: 146 - DNA, 119 bp, Homo sapiens
gcttttgctgaacatcaagtggtgagccaggactcaaagccagatcttcttgtttccctgttaggtgtttgtagcacaactggtatctgcagactatgctgctggaagggctagccgtc

SEQ ID NO: 147 - DNA, 20 bp, Homo sapiens
gcttttgctgaacatcaagt

SEQ ID NO: 148 - DNA, 19 bp, Homo sapiens
ccttccagcagcatagtct

SEQ ID NO: 149 - DNA, 161 bp, Homo sapiens
actgtcctagaaaatccaggatgtgcagtgatcatgtatgaatgcatggacctgcacacacaggagtgaacaaaagacccacccctgccaggtcaccactcatatctcaccccagcccacgctagctcacactcctccccacacaccactgacctcatcat

SEQ ID NO: 150 - DNA, 18 bp, Homo sapiens
aaatccaggatgtgcagt

SEQ ID NO: 151 - DNA, 19 bp, Homo sapiens
atgatgaggtcagtggtgt

SEQ ID NO: 152 - DNA, 124 bp, Homo sapiens
cacatcacagatcatagtaaatggctttaattttttaacgaaatctcactactgcaaatgcattgttgtcctagctaatgaatgcatagagtattgcctgcaaaataataattgagattctatt

SEQ ID NO: 153 - DNA, 22 bp, Homo sapiens
catcacagatcatagtaaatgg

SEQ ID NO: 154 - DNA, 21 bp, Homo sapiens
aattattattttgcaggcaat

SEQ ID NO: 155 - DNA, 151 bp, Homo sapiens
ttatcctccacatcctcatgaggcaaacacctttcctaccttaccgctccccagtggcctccctgttgccttcttattcaagactaagactctctagaatgttctttatcctgagtccagctgattgtctatactaatatcagtacggggt

SEQ ID NO: 156 - DNA, 20 bp, Homo sapiens
catgaggcaaacacctttcc

SEQ ID NO: 157 - DNA, 22 bp, Homo sapiens
gctggactcaggataaagaaca

SEQ ID NO: 158 - DNA, 145 bp, Homo sapiens
agggtgcagcactttattatggaagcctgagctgactaatacaggtgtctctatatctcactgagggaaagtgacaggaaagtaagaaccatttatgtccaagagtccagaggagtcaaccagattctgggggaaaagaaggtac

SEQ ID NO: 159 - DNA, 20 bp, Homo sapiens
tggaagcctgagctgactaa

SEQ ID NO: 160 - DNA, 20 bp, Homo sapiens
ccttcttttcccccagaatc

SEQ ID NO: 161 - DNA, 154 bp, Homo sapiens
tgagaatttaggagaacagaagatcagagggctgcacaggctaaactagacaatgagcccatgcaagtaagttaagaggagaagcgggtaagtatgcacctgctttgtctaggtgaccagcaagcatttagcaatagtcttttcaaaacaacag

SEQ ID NO: 162 - DNA, 22 bp, Homo sapiens
ttaggagaacagaagatcagag

SEQ ID NO: 163 - DNA, 22 bp, Homo sapiens
aaagactattgctaaatgcttg

SEQ ID NO: 164 - DNA, 106 bp, Homo sapiens
aaacaggcaaaataagcgtagggctgtgtgtgcaacagttaatcataaagccatcaccaggagacgtcactgggcgccttctggagtctatccgtcctaactttgc

SEQ ID NO: 165 - DNA, 20 bp, Homo sapiens
taagcgtagggctgtgtgtg

SEQ ID NO: 166 - DNA, 21 bp, Homo sapiens
ggacggatagactccagaagg

SEQ ID NO: 167 - DNA, 105 bp, Homo sapiens
gaatgaccttggcacttttatcaaacatcaactggccacacacaggtgagtctacttctggacacttatcctgttccattcatctgtatatctctatccttacac

SEQ ID NO: 168 - DNA, 23 bp, Homo sapiens
gaatgaccttggcacttttatca

SEQ ID NO: 169 - DNA, 27 bp, Homo sapiens
aaggatagagatatacagatgaatgga

SEQ ID NO: 170 - DNA, 129 bp, Homo sapiens
ctgctggaataggctgcttggccatgttcttggaagctaccaccatatcaaggtaatttcccacacaacattccagcccctgctttcctctctggccttatctagggccattccccaactcaggtgaat

SEQ ID NO: 171 - DNA, 20 bp, Homo sapiens
ggccatgttcttggaagcta

SEQ ID NO: 172 - DNA, 20 bp, Homo sapiens
ttcacctgagttggggaatg

SEQ ID NO: 173 - DNA, 138 bp, Homo sapiens
acctttgttccatgcaccgcgcaaatacctgggaacccttattgcccaactcaagagccagagtcctctgtcatcattttgcctctctcctaagtgagaggactgagtgcagacttggtgtttgtgggtgaggcatgt

SEQ ID NO: 174 - DNA, 18 bp, Homo sapiens
catgcaccgcgcaaatac

SEQ ID NO: 175 - DNA, 19 bp, Homo sapiens
atgcctcacccacaaacac

SEQ ID NO: 176 - DNA, 159 bp, Homo sapiens
ctcctgagtccaagcccttctcactcacctctttcttgaactaatttcttcctgtttttttccagtcctcccttctgttcatgtctctcctctgcacacttccattttgtggttcagaaaatgtcaccgtcccagtcacacttgccttatggctgttgt

SEQ ID NO: 177 - DNA, 20 bp, Homo sapiens
tccaagcccttctcactcac

SEQ ID NO: 178 - DNA, 20 bp, Homo sapiens
ctgggacggtgacattttct

SEQ ID NO: 179 - DNA, 132 bp, Homo sapiens
cccaggaagagtggaaagattaacctttgtgagccaaaccagtgacacttgattacttgacagaactaatccttctgtcctgatgacagaacttcaactacacaggtacatgcaagctaatatctgttgtaa

SEQ ID NO: 180 - DNA, 21 bp, Homo sapiens
cccaggaagagtggaaagatt

SEQ ID NO: 181 - DNA, 21 bp, Homo sapiens
ttagcttgcatgtacctgtgt

SEQ ID NO: 182 - DNA, 165 bp, Homo sapiens
gcctggcaagctagatggggtgaattttcacctgccacagccgcaagtcaaagccaccggcttctctcttctccctcccattgctcctgacagccagggttaatattttgcctcatgtaaacagggaggcatccacccgagaatctcccctcagcccacataagc

SEQ ID NO: 183 - DNA, 20 bp, Homo sapiens
agctagatggggtgaatttt

SEQ ID NO: 184 - DNA, 18 bp, Homo sapiens
tgggctgaggggagattc

SEQ ID NO: 185 - DNA, 160 bp, Homo sapiens
atcaagctaattaatgttatctatcacttcacatagttcaacctttttttgtggtgagagtactgaagatctactctcttagcaattttcaaatctaaaatacattattattaacacagtcactgtgccgtacgttagctctgaggaccttattcatttt

SEQ ID NO: 186 - DNA, 22 bp, Homo sapiens
atcaagctaattaatgttatct

SEQ ID NO: 187 - DNA, 20 bp, Homo sapiens
aatgaataaggtcctcagag

SEQ ID NO: 188 - DNA, 124 bp, Homo sapiens
tttaatctgatcattgccctatgaggtagggagtattctgattcccattttataaataaggaacccgaggcttagagagcatcagtgacttgttcaaggtcacccacagctgtcaagtgacaga

SEQ ID NO: 189 - DNA, 21 bp, Homo sapiens
tttaatctgatcattgcccta

SEQ ID NO: 190 - DNA, 18 bp, Homo sapiens
agctgtgggtgaccttga

SEQ ID NO: 191 - DNA, 128 bp, Homo sapiens
tgtcccaccattgtgtattaggtttgtagagcgtagacaacttgcctttttagtttgtaggtttctgtatcaagagaagatgtgtgtgggcctaacctagattacaggatcctggacttcaagtctga

SEQ ID NO: 192 - DNA, 20 bp, Homo sapiens
tgtcccaccattgtgtatta

SEQ ID NO: 193 - DNA, 20 bp, Homo sapiens
tcagacttgaagtccaggat

SEQ ID NO: 194 - DNA, 128 bp, Homo sapiens
tcatttgctaaggtcggatagctcctaattggcaaagtcacgatgggatcccagggattctgaggatgaagcctgtgtttaataactattatgccaagtgagcattttcaaatatatgagagaaatta

SEQ ID NO: 195 - DNA, 19 bp, Homo sapiens
catttgctaaggtcggata

SEQ ID NO: 196 - DNA, 20 bp, Homo sapiens
tatttgaaaatgctcacttg

SEQ ID NO: 197 - DNA, 159 bp, Homo sapiens
cattgcttcaggggtgttagttttgtgttcacaactagattataaactcctcttgcattcctgatggcagtgacttgaaggcatttatttgaagaataatagacatacagaaaggggcacatgtcataaaggtacagctggacgacttttcacaaagtg

SEQ ID NO: 198 - DNA, 20 bp, Homo sapiens
gcttcaggggtgttagtttt

SEQ ID NO: 199 - DNA, 20 bp, Homo sapiens
ctttgtgaaaagtcgtccag

SEQ ID NO: 200 - DNA, 117 bp, Homo sapiens
gagaggatggtgccatcatggaaagcatggggcagtcatggagatgacggagtagctcatggagaagataatgccatcatggaaggcatagtgcagtcatggagatgatggtgcagc

SEQ ID NO: 201 - DNA, 18 bp, Homo sapiens
ccatcatggaaagcatgg

SEQ ID NO: 202 - DNA, 20 bp, Homo sapiens
tcatctccatgactgcacta

SEQ ID NO: 203 - DNA, 167 bp, Homo sapiens
atggggcagtcatggagatgacggagtagctcatggagaaaataatgccatcatggaaggcatagtgcagtcatggagatgatggtgcagctcatggagaagatggtgccatcatggaaggcatggtgcaatcatggagtagacagtgcagctgggccaagattctc

SEQ ID NO: 204 - DNA, 20 bp, Homo sapiens
gagatgacggagtagctcat

SEQ ID NO: 205 - DNA, 18 bp, Homo sapiens
cccagctgcactgtctac

SEQ ID NO: 206 - DNA, 128 bp, Homo sapiens
gatgtgcctctcttgttccaatcacaggacaggggtataactaggggcactgtctatactggctgcactctggccagtgctgtcccaggtagattcatcagggtctagagcttcagctaacagcatga

SEQ ID NO: 207 - DNA, 20 bp, Homo sapiens
tcttgttccaatcacaggac

SEQ ID NO: 208 - DNA, 20 bp, Homo sapiens
atgctgttagctgaagctct

SEQ ID NO: 209 - DNA, 109 bp, Homo sapiens
ttttattcattaagttgaaagctcctaaagcagagggaccatatttttatgtcccaactctccttaaggccttgcctatgatagcacatctcttcaatagaattgtcct

SEQ ID NO: 210 - DNA, 20 bp, Homo sapiens
tgaaagctcctaaagcagag

SEQ ID NO: 211 - DNA, 20 bp, Homo sapiens
ttgaagagatgtgctatcat

SEQ ID NO: 212 - DNA, 163 bp, Homo sapiens
cacataactaataaatttgtaagtatgtgcaacggctcacacttgcttccagaatggcacctaaaaaacagatttacctctccccaaattcagatatggaattaaatgtaatgtcaggaaaactgtctaagagttggaaatgggaaaaaaatgttcttttggt

SEQ ID NO: 213 - DNA, 21 bp, Homo sapiens
aatttgtaagtatgtgcaacg

SEQ ID NO: 214 - DNA, 207 bp, Homo sapiens
aaagaccagcttttagctgaacatcagggctgccttcagagtttaattaccgccctccccatggggccaaatgagccatcgactcctcccaagggggttcggcttggtactgatctttaagtaagtaaacgctaaaccagctcatcttaaagcgcccacatctgatttcctgctctgctgcaagacagtaggtgactggtaatgacc

SEQ ID NO: 215 - DNA, 20 bp, Homo sapiens
agggctgccttcagagttta

SEQ ID NO: 216 - DNA, 20 bp, Homo sapiens
gcgctttaagatgagctggt

SEQ ID NO: 217 - DNA, 192 bp, Homo sapiens
actctgctcccagtgtgaacatggggaaagttgattaaactctctgacttcagattcctcatgtaaaatgtggggaaacagctctgacttaatggtgtcactgtgaggagtaaatgaggtagcatatttaaaggattttgtatagtgctggtgacagtaaccagccaatagatgatatagctagtaatagca

SEQ ID NO: 218 - DNA, 20 bp, Homo sapiens
tgaacatggggaaagttgat

SEQ ID NO: 219 - DNA, 22 bp, Homo sapiens
tcaccagcactatacaaaatcc

SEQ ID NO: 220 - DNA, 170 bp, Homo sapiens
cttcactgaccacttccttaactgtccactccgaaacaccccttcttcctgttcttccaatacaccaaactctttcttgcctctgtgtgcttgcccatgctgttccttctggcttcttccttcacattcaagtcttgacttagatgtcacttgccaagggagaccttgga

SEQ ID NO: 221 - DNA, 21 bp, Homo sapiens
acttccttaactgtccactcc

SEQ ID NO: 222 - DNA, 19 bp, Homo sapiens
ccttggcaagtgacatcta

SEQ ID NO: 223 - DNA, 181 bp, Homo sapiens
aaacatcccaatagacaaaactccaagaagagtcaaaacaagaataaagtacaggtcatcttttcttttgcactcctgacagcactttgtacatggtaataataatctaccaattaactacataagccacatggttttatcatagtgtgaagctttgtatccagaaaggagagaaggctcc

SEQ ID NO: 224 - DNA, 22 bp, Homo sapiens
ccaagaagagtcaaaacaagaa

SEQ ID NO: 225 - DNA, 21 bp, Homo sapiens
tctcctttctggatacaaagc

SEQ ID NO: 226 - DNA, 149 bp, Homo sapiens
ggcagaggcatggggtgcatagggatatggggtgggccagtttgctcctcagaccagaaggggtgcaggactccccccgatcaggatcatggagaaaggtgtggacagaggaagggagggagggagaaatggcagctgccctgcagtgg

SEQ ID NO: 227 - DNA, 20 bp, Homo sapiens
ttgctcctcagaccagaagg

SEQ ID NO: 228 - DNA, 20 bp, Homo sapiens
ctcccttcctctgtccacac

SEQ ID NO: 229 - DNA, 165 bp, Homo sapiens
cagggactaagtgtctctgacaatacattcagccactactacagtatgaagccagcccctcatccccaccttcagagacccctggtgcctcagattcctcggccattctggagctgctgtgcccgaggcttgtgtagttggagatcattttggcagtcagtgctg

SEQ ID NO: 230 - DNA, 22 bp, Homo sapiens
tgtctctgacaatacattcagc

SEQ ID NO: 231 - DNA, 20 bp, Homo sapiens
ctgactgccaaaatgatctc

SEQ ID NO: 232 - DNA, 181 bp, Homo sapiens
cctgtctccgtgcgtgaaagccggctccaaagtgccttctgtcctatctgccttccgcacctggctttcctgaaagaaagaaaacgcgtggcttatcttttcacggcacgccaccttcactctcactttttcttttctaataaatacctctggatgggttagtggtaatctctcctcaaac

SEQ ID NO: 233 - DNA, 20 bp, Homo sapiens
gctccaaagtgccttctgtc

SEQ ID NO: 234 - DNA, 20 bp, Homo sapiens
ccactaacccatccagaggt

SEQ ID NO: 235 - DNA, 198 bp, Homo sapiens
gggagcacaacctaggcccctcctggggaggtggtggagtcagaatcacgtaagagacaaagttccagtccctcagtgccggctccattgtcccctggacttcccttacaaaccacagatgcaaagagagcacttctcggaatctccacacagccacggtggagcactcaacccacgcgaccctcgggcgcaggtgct

SEQ ID NO: 236 - DNA, 20 bp, Homo sapiens
ctggggaggtggtggagtca

SEQ ID NO: 237 - DNA, 20 bp, Homo sapiens
gagtgctccaccgtggctgt

SEQ ID NO: 238 - DNA, 161 bp, Homo sapiens
cctgagaagcttccagcaaagcaccagcacgaaccgccccacctccccacctccccgcaagcgttgccgggactgacagattacagagctctggtccctctgcactcctgctctgccacccccagggtgtcagaatgtgccccccacacagtttccaaaag

SEQ ID NO: 239 - DNA, 18 bp, Homo sapiens
aaagcaccagcacgaacc

SEQ ID NO: 240 - DNA, 18 bp, Homo sapiens
ggggcacattctgacacc

SEQ ID NO: 241 - DNA, 162 bp, Homo sapiens
atggagctgctgcgccggcctgagctctgatccctcctccgacccagcctcaccctgcaagcagcaccatgtggggctcagaatggggatcttaagggaccctccccacaacctcccgataagcctttccacggagggcccaagcggagacaggagaacact

SEQ ID NO: 242 - DNA, 19 bp, Homo sapiens
ctgagctctgatccctcct

SEQ ID NO: 243 - DNA, 18 bp, Homo sapiens
ttctcctgtctccgcttg

SEQ ID NO: 244 - DNA, 194 bp, Homo sapiens
actttcagaatgtgctgccttccacgtgtgaaccagactgagctcctttctgccactgatgttgaattgtccatttgctcacatcagtgtccacgtggcaaatccacagggcgtgggtgggatcctgcagtctagacaaagccaaggagcaccgctggaggccacgttgggcttcccaatccacatgcaaaccc

SEQ ID NO: 245 - DNA, 20 bp, Homo sapiens
cctttctgccactgatgttg

SEQ ID NO: 246 - DNA, 19 bp, Homo sapiens
ttgcatgtggattgggaag

SEQ ID NO: 247 - DNA, 213 bp, Homo sapiens
tctccagccagcgtgtcacaaagccgctcacctgctcgtgtgagtgtctgaatgcacgtgtttgagtgtcagaggcgtgtgaaccacagcaactcaatcttgaataggggctgggtaaagtgaggctgagacctcccggggctgcattcccagatggttaaggcattctaagtcacaagatgagataggaagttcgcacaagacactggtcat

SEQ ID NO: 248 - DNA, 20 bp, Homo sapiens
tcacctgctcgtgtgagtgt

SEQ ID NO: 249 - DNA, 20 bp, Homo sapiens
ccttaaccatctgggaatgc

SEQ ID NO: 250 - DNA, 152 bp, Homo sapiens
ttgagtcctcttaagtagttactatagtggagaacttgagtcattctttgtagcgtgcttcgtagagcagcgtgtttgttagaaggatttgttaatcctgtatagggtctttacgaaggctgttttcatggaagcttctctttgttgactcc

SEQ ID NO: 251 - DNA, 22 bp, Homo sapiens
tggagaacttgagtcattcttt

SEQ ID NO: 252 - DNA, 19 bp, Homo sapiens
ggagtcaacaaagagaagc

SEQ ID NO: 253 - DNA, 174 bp, Homo sapiens
cattctctccagctgcaaactttcttcaactttcctaaattcttactaaattcagaggaataggataaagatcacttagagaaagggtgcttatggacatagcctgagtttcctttaacctctctgcaatgggtgcttttaactagcttctacatggcaagctgtttcagtttg

SEQ ID NO: 254 - DNA, 21 bp, Homo sapiens
ctttcttcaactttcctaaat

SEQ ID NO: 255 - DNA, 19 bp, Homo sapiens
ttgccatgtagaagctagt

SEQ ID NO: 256 - DNA, 175 bp, Homo sapiens
ggacatctggaactgcaccagcacagaaccgacacgttgttactcatcgtcactcggcagggctgaagaccaccagaactcatgacaggcagacgtgcctggcccagttgaggatgtagcttcagagccaagcgccagtcctgttggccacgtgggctgggggcaggatagacca

SEQ ID NO: 257 - DNA, 19 bp, Homo sapiens
accagcacagaaccgacac

SEQ ID NO: 258 - DNA, 18 bp, Homo sapiens
aacaggactggcgcttgg

SEQ ID NO: 259 - DNA, 182 bp, Homo sapiens
ggctggttctgcccttgggaggtggttcctttggctggaccagaatgtctgaagatgatcaggagagggccaagggttggggggtgccccatgtgcaccctgagaattgcaccaggcacagtgagcaacttcagccctccttgtgcagagctgcagcgtacagtgccagccctcgctggccc

SEQ ID NO: 260 - DNA, 20 bp, Homo sapiens
cttgggaggtggttcctttg

SEQ ID NO: 261 - DNA, 18 bp, Homo sapiens
ctgcacaaggagggctga

SEQ ID NO: 262 - DNA, 155 bp, Homo sapiens
gttctcactttactgagaaacctggcagcttctcaggccaccgcccaggtcacctgctcaccagcaacgtgaaccacaggaactgaggctgtgcgggaggcggctctgctctgtgctgggcccccctcctcctcactcaccctcttcagtcaaag

SEQ ID NO: 263 - DNA, 20 bp, Homo sapiens
tactgagaaacctggcagct

SEQ ID NO: 264 - DNA, 20 bp, Homo sapiens
ctttgactgaagagggtgag

SEQ ID NO: 265 - DNA, 180 bp, Homo sapiens
ttagtattattattttcatatatattttttataataatcatatattcaattttatcatcaagaaaaaagttttaaaattcaaaatcctttcatgtgcactgttttaaacttaggtagaagaaaaaaagtcactgaaaatccaagatgtaataaacaggcccaacaaaggccaacaaactt

SEQ ID NO: 266 - DNA, 20 bp, Homo sapiens
ttcaattttatcatcaagaa

SEQ ID NO: 267 - DNA, 20 bp, Homo sapiens
ttgggcctgtttattacatc

SEQ ID NO: 268 - DNA, 196 bp, Homo sapiens
agggaacatggccttgcccacacagatttcagacatctggctccagaactgtgggaggacacatttctgttgtttagaactgcatgttttttatactttgttatggctgccctaggcaactaatacagatattattttccacttctgaacttagcaaaatatttttaaaatgaaaattcttaaatgttggcacagt

SEQ ID NO: 269 - DNA, 20 bp, Homo sapiens
ttgcccacacagatttcaga

SEQ ID NO: 270 - DNA, 22 bp, Homo sapiens
tgctaagttcagaagtggaaaa

SEQ ID NO: 271 - DNA, 120 bp, Homo sapiens
ctggataaaggatgctacacgtccctggtgggacagagcaggacggcaggggatttcattacgccactcagaatggcaggcaattgaaaaaacttataaattgtttatttccagaatttt

SEQ ID NO: 272 - DNA, 20 bp, Homo sapiens
ggataaaggatgctacacgt

SEQ ID NO: 273 - DNA, 20 bp, Homo sapiens
aaaattctggaaataaacaa

SEQ ID NO: 274 - DNA, 224 bp, Homo sapiens
tgtcagtggtgtaatccgactgtgaaagatcagtctaacaaaacagcggggagagagagggctgaatcagagcaactaggtccaaagccgagggaaccaccaacagatcccctggtgacccaacaagaaatgctcacagtctggacccagtcagagtctgcaggacacagcagacattctggaagttacaacagccaggagcaagaggacgcatggcctgactg

SEQ ID NO: 275 - DNA, 20 bp, Homo sapiens
gggagagagagggctgaatc

SEQ ID NO: 276 - DNA, 21 bp, Homo sapiens
gctcctggctgttgtaacttc

SEQ ID NO: 277 - DNA, 174 bp, Homo sapiens
cgccagagcaccccttctcagaacagaaagcgtctctacaaagtgatccggaagtgagtgtgtgagggcgctgcgtcctccctgctccccttggagttgccctttcttgctcagatctgggtgccttggccttgtcctgggcccttccgcagcccccggggtgatccccgctag

SEQ ID NO: 278 - DNA, 19 bp, Homo sapiens
ccagagcaccccttctcag

SEQ ID NO: 279 - DNA, 18 bp, Homo sapiens
ggaagggcccaggacaag

SEQ ID NO: 280 - DNA, 201 bp, Homo sapiens
tatcttacggatttgtcaacatcatttgagaagaagtccataggctcagcagatttttatgccaggtgggccatggcataaaaatgtgaagaatgtgctcacttagacaatacctgtgctaaaattggaacaatacagagaagattagcaaattaaaacaatgttaggaagtcagtgtggtgaggtacggtgcctcatgcc

SEQ ID NO: 281 - DNA, 21 bp, Homo sapiens
cagcagatttttatgccaggt

SEQ ID NO: 282 - DNA, 20 bp, Homo sapiens
caccgtacctcaccacactg

SEQ ID NO: 283 - DNA, 275 bp, Homo sapiens
aggcagggccctccttgccacatgtaaagctgcacagagcggtcactatatgtgtttccatatttgcaatccaaccaccaccaactgagtgtgcgtcctgatcagccgagcctgcccacggtggccacaggccctctacattctaatctcgagagcctgagcatgtacaaattaaacgaagcaaaacgacaccacccagttctggccgtactataggaggtttccaggaagggtttgtgaacataaacataagctaggtaacactcctttctgaa

SEQ ID NO: 284 - DNA, 20 bp, Homo sapiens
aaccaccaccaactgagtgt

SEQ ID NO: 285 - DNA, 20 bp, Homo sapiens
cctcctatagtacggccaga

SEQ ID NO: 286 - DNA, 230 bp, Homo sapiens
tcagagcatcgcctcagtggccatcaatagctcgggggactggattgcttttggctgttcaggtttgtccccagcctgggtggtagagatggactccccattagggaccagtgctgcccggctacaggcttacttgacagccacccactgggggtgccctcccctcccccagttgtcttccatggggtgccctctcccccagccgcctttcagaaggggccctcccctcc

SEQ ID NO: 287 - DNA, 20 bp, Homo sapiens
tggattgcttttggctgttc

SEQ ID NO: 288 - DNA, 20 bp, Homo sapiens
caccccatggaagacaactg

SEQ ID NO: 289 - DNA, 199 bp, Homo sapiens
ctcatgcttacatccttagctgatcattaaactttgtgaccatttcatgctcactgctttcttgcccgggagctaatggtgaggaaaggtcactgggaaccagcgcaccaacctcagacatcgattttgttccagccttttttcctgggcaggggtggctatcacctgctggtaggcagcggcaggcccactgtcctgc

SEQ ID NO: 290 - DNA, 21 bp, Homo sapiens
ttaaactttgtgaccatttca

SEQ ID NO: 291 - DNA, 18 bp, Homo sapiens
taccagcaggtgatagcc

SEQ ID NO: 292 - DNA, 181 bp, Homo sapiens
tgacagaaaagtctcagagcagtgccttctgagctcttctacaccaagcaggcagaatgttcactgctaatgaggctggagctggtccccagcagtggtaggaagcttccaacaggctcaggctgtgggtgcttgcaggggcacagtgtgacggccacgggcctcagagctctggtgggct

SEQ ID NO: 293 - DNA, 20 bp, Homo sapiens
gacagaaaagtctcagagca

SEQ ID NO: 294 - DNA, 18 bp, Homo sapiens
caagcacccacagcctga

SEQ ID NO: 295 - DNA, 187 bp, Homo sapiens
acatctttctcaaataaagataacagcgatgtattttcacaaaagcaagagcttagaaagtactccacccaggtatccctcttggaaaaaatacttaaggaaatatgacaaatggcaaagtgattgttatggatggaatgtttgtatcctcccaaaattcacatgttgagaccctaattccaatatg

SEQ ID NO: 296 - DNA, 20 bp, Homo sapiens
attttcacaaaagcaagagc

SEQ ID NO: 297 - DNA, 20 bp, Homo sapiens
ttgggaggatacaaacattc

SEQ ID NO: 298 - DNA, 180 bp, Homo sapiens
aggggcattctacaaaacacccaaccggtcaaggtcgctgaggccaaggagagattgggcaaccgtcacaaaccagagaagccgaggagacctttcagccaacgccatgtggggtcctgagcaggacccaccggaagttggtgcagctgcctaaagaccgtcctggctgagaagaaacag

SEQ ID NO: 299 - DNA, 18 bp, Homo sapiens
aaggagagattgggcaac

SEQ ID NO: 300 - DNA, 19 bp, Homo sapiens
gtttcttctcagccaggac

SEQ ID NO: 301 - DNA, 228 bp, Homo sapiens
tggccctgacctgccagagctgttggcctccagctggcgggtaaaacccacggccttctcagaacaggtttctcaacacatgagacagaacacaccagacttccaaggggaacacctggatggagctggttacccagatcgttcaacaccgaggggcagcggcttgagggtctttccacgaaggcttggattaacaagaggagcasrggtctctccaggatgggccca

SEQ ID NO: 302 - DNA, 20 bp, Homo sapiens
catgagacagaacacaccag

SEQ ID NO: 303 - DNA, 20 bp, Homo sapiens
tcttgttaatccaagccttc

SEQ ID NO: 304 - DNA, 223 bp, Homo sapiens
ccacccagtgtcacgtcacggccccggcacgccatccacggaccctggatggagcccagctgcctccaggagcgcagtttaactacaaaggagccctggctgcccgccccgcccagacgcactgacctgttgttctctgtggctgctgatggcccatccccaaccactggtgactcttccctggggccccaagctcagcccctaaccccctgttgctggaagt

SEQ ID NO: 305 - DNA, 18 bp, Homo sapiens
cacggaccctggatggag

SEQ ID NO: 306 - DNA, 18 bp, Homo sapiens
cagggaagagtcaccagt

SEQ ID NO: 307 - DNA, 186 bp, Homo sapiens
cagaggactgggctgcggggtcaggaatgggcacacttcctaactgcaggacactctaagggctttggtcatgcacacgcagccaagagaaggtgtcgctgacacacagccttccaggagcggacttggagacctcgccaaggaccaggactccccagcactcacactcccttaggcgctgaagtc

SEQ ID NO: 308 - DNA, 20 bp, Homo sapiens
actctaagggctttggtcat

SEQ ID NO: 309 - DNA, 20 bp, Homo sapiens
ctaagggagtgtgagtgctg

SEQ ID NO: 310 - DNA, 185 bp, Homo sapiens
gaagaggacaacacggggctgtctgcagagcacctgccacgcgccaggctctgtgtccacaagcacggcggctgctcccacatgacagagctcgtgcggcagctccaggactgtctggtgccagagccccagctctccgccagccccaggccactgtgcgaggccctcagtgaagagggggccgt

SEQ ID NO: 311 - DNA, 18 bp, Homo sapiens
cgccaggctctgtgtcca

SEQ ID NO: 312 - DNA, 18 bp, Homo sapiens
ggccccctcttcactgag

SEQ ID NO: 313 - DNA, 173 bp, Homo sapiens
tctaaataatgttaatgatcaaatttagtcagatctcaatcttcatatgttagttgccttcttaataaatattctgttttctttatcgttctttatttgtatctccaccttcatttctgattaaattaagaagttttgtctcttccatttaataattaatgtatttaataacc

SEQ ID NO: 314 - DNA, 22 bp, Homo sapiens
tttagtcagatctcaatcttca

SEQ ID NO: 315 - DNA, 22 bp, Homo sapiens
aatggaagagacaaaacttctt

SEQ ID NO: 316 - DNA, 194 bp, Homo sapiens
cacactccacactggccccacgcgggtggcgaaggactcagccagagcctggcaggatcctggggtgtctatttccaaggaatgttctggaagaaacatacacacatacttgtttgccagatttacctgtgtggtcttccagatgagaagcagcctgtgtcactccataagggagagtgcgtgcagcattgaga

SEQ ID NO: 317 - DNA, 18 bp, Homo sapiens
gaaggactcagccagagc

SEQ ID NO: 318 - DNA, 20 bp, Homo sapiens
ctctcccttatggagtgaca

SEQ ID NO: 319 - DNA, 225 bp, Homo sapiens
aagaaactcccaaggaacgcattgtcccaagttgctgcaccagtcagtgtacattcccacaaacagtgcatgagagttcctgttgcttgtgaaataaatggtcagcattcagtgttgtcagcttttaaaattttctcctttctagtgggcatgtaatggtctcacattatagttttaatttgcattttcctggtgacatgtgatacggaaccttcctcccatgct

SEQ ID NO: 320 - DNA, 19 bp, Homo sapiens
accagtcagtgtacattcc

SEQ ID NO: 321 - DNA, 19 bp, Homo sapiens
ggaaaatgcaaattaaaac

SEQ ID NO: 322 - DNA, 170 bp, Homo sapiens
gtgcaatttaattacaaacgcttaaatggggaggtcaggggcagagggatgatgtcacaaacacacccacgtgtgcttggtgcaaaacagtaaaacaaacagcaagaaggtccatgaaggaaagatcgcctctgtcagtgggagtaatgagagtggctgatggacaggtg

SEQ ID NO: 323 - DNA, 20 bp, Homo sapiens
cgcttaaatggggaggtcag

SEQ ID NO: 324 - DNA, 20 bp, Homo sapiens
cctgtccatcagccactctc

SEQ ID NO: 325 - DNA, 189 bp, Homo sapiens
acgccaagcaggagatgccagacacagagtccatcctgagagagtctgttcctgtccaagctcagaaacacaggaagccacctgtgctgtagcagcacacggagatgcatcctttctggtccaccccacggccctcattgcagtcagggatcctctcccagaaagtccctgctgccagcccctgccctt

SEQ ID NO: 326 - DNA, 20 bp, Homo sapiens
agcaggagatgccagacaca

SEQ ID NO: 327 - DNA, 20 bp, Homo sapiens
ggtggaccagaaaggatgca

SEQ ID NO: 328 - DNA, 170 bp, Homo sapiens
catgagaaagactttgttcccatgagaacaacaagagaaactcaaacaaaattaaaattgtacttttctaaaagaccggggtgggggtcgtggtcaggcagcagcatgaagaaagccttgagaactgaattccagaaagaaacaagcataggcaagaaagagagatgaca

SEQ ID NO: 329 - DNA, 22 bp, Homo sapiens
cccatgagaacaacaagagaaa

SEQ ID NO: 330 - DNA, 20 bp, Homo sapiens
ctctttcttgcctatgcttg

SEQ ID NO: 331 - DNA, 167 bp, Homo sapiens
aagatttagaacagctgaagcagcgagaaaaaacccagcatgagtcagaactggagcaactgaggatttattttgaaaagaagttaagggatgctgagaaaacttaccaagaagacctaaccctgttacagcagaggctgcagggggcgagggaagatgctcttctg

SEQ ID NO: 332 - DNA, 21 bp, Homo sapiens
cagctgaagcagcgagaaaaa

SEQ ID NO: 333 - DNA, 19 bp, Homo sapiens
cccctgcagcctctgctgt

SEQ ID NO: 334 - DNA, 174 bp, Homo sapiens
gggaaactgacttggcttttgcaagggtcattgcttcctgatgcatgtttaactgtcctgtgttcactttgttgccgcaggtttttagaggaacgtaaagagatcaccgagaaattcagtgcggaacaagatgccttcctgcaggaggcccaggagcagcatgcccgtgagctg

SEQ ID NO: 335 - DNA, 22 bp, Homo sapiens
gggaaactgacttggcttttgc

SEQ ID NO: 336 - DNA, 20 bp, Homo sapiens
ggcatcttgttccgcactga

SEQ ID NO: 337 - DNA, 175 bp, Homo sapiens
ccctgcacactgacctgcatgccctcgtcacctgcactctgcatgctcaccatctgacggactcctgcgacgggcatgggaaggtcgccgccgccggcagccttgcgagcactttggatgtgtgcacccggcatgccaggcccgagtcaacagactggccgaccttggcgtcctg

SEQ ID NO: 338 - DNA, 20 bp, Homo sapiens
gccctcgtcacctgcactct

SEQ ID NO: 339 - DNA, 20 bp, Homo sapiens
ccaaggtcggccagtctgtt

SEQ ID NO: 340 - DNA, 155 bp, Homo sapiens
tttattgctgagtggtattccattttatgggtccattatagtttatttgtccagacacttcatggaaagacatcagtgtttcctgtttttcaatcataaattgatgtttaattttaaaattttggaattgtagaagaaatgcaattcttttttcc

SEQ ID NO: 341 - DNA, 22 bp, Homo sapiens
tgggtccattatagtttatttg

SEQ ID NO: 342 - DNA, 22 bp, Homo sapiens
tgcatttcttctacaattccaa

SEQ ID NO: 343 - DNA, 208 bp, Homo sapiens
ctttggtgcagaatcatgctgcaggcaaggtgggcccacctccctggaatttcatcccccccgtcagttaaacccatggtggttttattttctaggccacctgatctgggaggaccacctccaagaaaagcagtcctatcgatgaacggtctaagttatggtgttatcagagtggatactgaagaaaagttgtcagtccttactgttc

SEQ ID NO: 344 - DNA, 20 bp, Homo sapiens
ggcccacctccctggaattt

SEQ ID NO: 345 - DNA, 22 bp, Homo sapiens
tccactctgataacaccataac

SEQ ID NO: 346 - DNA, 232 bp, Homo sapiens
atcacctggtttggtgcatcctcgcagaaagagagccatacagtgaagtggaaacacacccaaaagctctgcaatattcctagaagttctcgaatctcctccttaacagagctgcagaagggaaacacagacaggaagcacctgtttgactcagacagcagccctaatgcagtgccactcaggagcattccctcatttgaagaccccccaattacatgaaattatcaacccc

SEQ ID NO: 347 - DNA, 20 bp, Homo sapiens
acacccaaaagctctgcaat

SEQ ID NO: 348 - DNA, 20 bp, Homo sapiens
caaatgagggaatgctcctg

SEQ ID NO: 349 - DNA, 240 bp, Homo sapiens
ggaactgcaggagatccctgctgccttccagttcatgggatgatggcctccacttctgcccctgtttgcttctcctttcaaatcttacatgaaggtatacagtttgaagaagccagtttgactccaatatctgtgcaatggaatactgctcattaaaaaggaattaaactattgatacacacaacatgggtgaagatcaaactgtctccttccctttgattcaagggaatctgagaaatg

SEQ ID NO: 350 - DNA, 19 bp, Homo sapiens
acttctgcccctgtttgct

SEQ ID NO: 351 - DNA, 21 bp, Homo sapiens
tgatcttcacccatgttgtgt

SEQ ID NO: 352 - DNA, 187 bp, Homo sapiens
tggagaaagttgttgcaaactgcccagagaccctgggagtcactccagttttctgaaacccagatatttcagtgcctcaggagagacaagtcctgaccttctctcctccagctctcccaggagataggcaagcccctaactccctaactaagcccttcagacctgaaatccattgagtggcttcttt

SEQ ID NO: 353 - DNA, 18 bp, Homo sapiens
gcaaactgcccagagacc

SEQ ID NO: 354 - DNA, 20 bp, Homo sapiens
ttagggagttaggggcttgc

SEQ ID NO: 355 - DNA, 184 bp, Homo sapiens
agggccatgggatgatgcaggtggagactggagtgctacagctgcaagcaaatacatttctgtgctgtgaagccacccatttggtggtactacgttaaaacagctctaggaaattaatacagatgttgcctgtatttttgtttctcatattactactcattgttttaatgatgactgttttatt

SEQ ID NO: 356 - DNA, 20 bp, Homo sapiens
aggtggagactggagtgcta

SEQ ID NO: 357 - DNA, 22 bp, Homo sapiens
agaaacaaaaatacaggcaaca

(19) United States
(12) Patent Application Publication
(10) Pub. No.: US 2010/0184043 A1
(43) Pub. Date: Jul. 22, 2010

(54) Detecting Genetic Abnormalities

(75) Inventors: Aoy Tomita Mitchell, Elm Grove, WI (US); Michael Mitchell, Elm Grove, WI (US)

Correspondence Address: MORGAN, LEWIS & BOCKIUS, LLP (SF), ONE MARKET SPEAR STREET TOWER, SAN FRANCISCO, CA, 94105, US

(73) Assignee: UNIVERSITY OF LOUISVILLE RESEARCH FOUNDATION, Louisville, KY (US)

(21) Appl. No.: 12/581,070

(22) Filed: Oct. 16, 2009

Related U.S. Application Data
(63) Continuation-in-part of application No. 11/713,069, filed on Feb. 28, 2007.
(60) Provisional application No. 61/106,435, filed on Oct. 17, 2008.
(60) Provisional application No. 60/777,865, filed on Feb. 28, 2006.

(51) Int. Cl. C12Q 1/68

(52) U.S. Cl. 435/6

(57) ABSTRACT

The present invention is directed to compositions and methods for detecting genetic abnormalities. The present invention encompasses methods and compositions for comparing alleles in a sample containing both maternal and fetal nucleic acids in order to identify genetic abnormalities.

### CROSS REFERENCE TO RELATED APPLICATIONS

This application claims the benefit of priority to U.S. Patent Application No. 61/106,435, filed Oct. 17, 2008, which is hereby incorporated by reference in its entirety. This application is a continuation-in-part of U.S. patent application Ser. No. 11/713,069, filed Feb. 28, 2007, which claims priority to U.S. Patent Application No. 60/777,865, filed Feb. 28, 2006, each of which is hereby incorporated by reference in its entirety.

### BACKGROUND OF THE INVENTION

A majority of pregnant women receive some kind of test, including maternal serum screening and/or an ultrasound test, to determine risks for common birth defects, such as those resulting from trisomy 13, 18, and 21 (Down Syndrome). Both the sensitivity and specificity of these common non-invasive screening tools are extremely poor. The best current non-invasive tests lead to a false positive rate between 7 and 20%. This high false positive rate often causes individuals to opt for invasive diagnostic tests, such as chorionic villus sampling (CVS) and amniocentesis. Such invasive tests each carry a fetal loss rate of 0.5%-1% and account for the loss of thousands of normal fetuses annually. However, prenatal diagnosis can be critical for management of a pregnancy with chromosomal abnormalities and localized genetic abnormalities, because an accurate and early diagnosis allows for interventional care before or during delivery and can prevent devastating consequences for the neonate. The development of a non-invasive test for genetic abnormalities that is sensitive and specific with low false-positive and false-negative rates would be of benefit to the field of molecular diagnostics.

### SUMMARY OF THE INVENTION

Accordingly, the present invention provides methods and compositions for detecting genetic abnormalities.

In one aspect, the present invention provides method for determining whether a fetus has at least one chromosomal abnormality. This method includes the step of comparing at least three alleles, and this comparing identifies the at least one chromosomal abnormality.

In a further aspect, the present invention provides a method for determining whether a fetus has at least one chromosomal abnormality. This method includes the steps of: (i) detecting a paternally-inherited fetal allele in a sample that includes both maternal and fetal nucleic acids, wherein that paternally-inherited fetal allele is not present in the maternal genome; (ii) detecting a first maternal allele and a second maternal allele in the sample; and (iii) comparing the paternally-inherited fetal allele to the first and second maternal alleles, where the comparing identifies the at least one chromosomal abnormality.

In an exemplary embodiment, comparing alleles according to the present invention includes detecting the number of molecules of each allele. In a further embodiment, detecting the number of molecules of each allele provides the allelic dosage of each allele.

In a still further exemplary embodiment, comparing alleles according to the present invention includes calculating a haplotype ratio. In a still further embodiment, the haplotype ratio is calculated according to the formula: HR=|P1−P2|/P3, where P1 is the number of molecules of a first maternal allele, P2 is the number of molecules of a second maternal allele, and P3 is the number of molecules of a paternally-inherited fetal allele.

### BRIEF DESCRIPTION OF THE DRAWINGS

FIG. 1 is a schematic illustration of an embodiment of an assay of the invention.

FIG. 2 is a schematic illustration of possible haplotypes in an exemplary embodiment of a tandem SNP.

FIG. 3 is a schematic illustration of an embodiment of an assay of the invention.

FIG. 4 is a schematic illustration of an embodiment of an assay of the invention.

FIG. 5A-B is a table of an exemplary list of tandem SNPs of the invention.

FIG. 6A-AO is a table of an exemplary list of tandem SNPs of the invention and primers directed to those tandem SNPs.

FIG. 7 provides a DNA melting map of a constant denaturant capillary electrophoresis target sequence covering a tandem SNP.

FIGS. 8A and B provides data of a haplotype ratio analysis.

FIG. 9A-C is a table of an exemplary list of tandem SNPs of the invention.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview of the Invention

The present invention is directed to methods and compositions for detecting genetic abnormalities. By “genetic abnormality” is meant any variation in one or more elements of an individual's genome in comparison to a general population. Genetic abnormalities can include without limitation chromosomal abnormalities, single point mutations, and any other variations that can result in changes in the levels of cDNA, RNA, mRNA, microRNA, and coding and non-coding RNA. Thus, the term “genetic abnormality” as used herein is interchangeable with the term “nucleic acid abnormality”. “Chromosomal abnormalities” can include without limitation aneuploidy (including without limitation trisomy 13 (Patau Syndrome), trisomy 18 (Edward Syndrome) and sex chromosome aneuploidies such as XXY (Klinefelter's Syndrome), subchromosomal abnormalities, gross deletions, gross insertions, large deletions, large insertions, copy number variants, copy number variation, repeat variations, structural alterations, small deletions (<20 bp), small insertions (<20 bp), indels, small indels (<20 bp), and the like. Genetic abnormalities can be inherited, arise somatically, occur as de novo events, occur clonally, etc.

In general, methods and compositions of the present invention can be used to analyze a sample comprising genetic material from two or more individuals. “Genetic material” includes without limitation DNA (including full or partial chromosomes, cDNA and genetic DNA) and RNA (including mRNA, tRNA, siRNA, micro RNA, coding and non-coding RNA). Methods of the present invention include comparing alleles contained in the sample to detect and quantify differences between the genetic material from the two or more individuals. In some embodiments, such differences can be used to identify genetic abnormalities in one or more of the individuals. Although the description of the present invention is primarily provided herein in terms of DNA, it will be appreciated that these aspects and embodiments of the invention also encompass RNA, and that all methods and compositions of the present invention can be applied to any nucleic acid.

In a specific aspect, methods and compositions of the invention are used to analyze a sample containing fetal and maternal genetic material. In a further aspect, methods and compositions of the invention are used to compare alleles contained in such a sample to determine if the fetal genetic material comprises a genetic abnormality. As used herein, “allele” is a variant form of a sequence at a particular region on a chromosome. The variants in the sequence can come as a result of single nucleotide polymorphisms (“SNPs”), combinations of SNPs, haplotype methylation patterns, insertions, deletions, and the like. An allele may comprise the variant form of a single nucleotide, a variant form of a contiguous sequence of nucleotides from a region of interest on a chromosome, or a variant form of multiple single nucleotides (not necessarily all contiguous) from a region of interest on a chromosome.

The term “comparing alleles” as used herein refers to any analysis that identifies similarities and differences among the alleles of interest. For example, comparing alleles may involve detecting and/or identifying the different alleles of interest in a sample. Comparing alleles may also involve detecting and quantifying the number of molecules of each of the different alleles of interest in a sample. Comparing alleles may further involve determining the relative number of molecules of each of the different alleles with respect to each other or with respect to a reference sample. In addition, comparing alleles may involve a comparison of the concentrations or relative concentrations of each of the alleles in a sample. As will be discussed in further detail herein, comparing alleles in a sample can detect and identify genetic abnormalities.

Although the present invention is primarily discussed in terms of a comparison between fetal and maternal genetic material, the methods and compositions described herein can also be applied to other applications in which a comparison of the genetic material between two or more individuals is desired. Embodiments of such extensions to the described methods and compositions are discussed in further detail herein and are encompassed by the present invention.

### II. Comparing and Quantifying Alleles

In one aspect, the present invention provides methods and compositions for comparing alleles in a sample.

A “sample” in accordance with the present invention may comprise any number of substances, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc.); as will be appreciated by those in the art, virtually any experimental manipulation may have been conducted on the sample.

In one aspect, samples of use in the present invention are obtained from a pregnant female. Such samples can include without limitation maternal blood, maternal urine, maternal sweat, maternal cells, or cell free DNA. In a further aspect, these maternal samples contain both maternal and fetal DNA.

Although the majority of the disclosure herein is directed to embodiments using DNA, it will be appreciated that any genetic material is contemplated for use and analysis in accordance with the present invention and is therefore encompassed by the present invention. In addition, the terms “chromosomal region” and “chromosome” are used interchangeably herein, and both refer to part or all of a chromosome unless otherwise specified.

In general, the present invention provides methods and compositions for comparing alleles in a sample. Comparing alleles in accordance with the invention includes detecting the identity of the different alleles present in the sample. Comparing alleles also includes detecting and/or quantifying the number of molecules of each allele of interest that is present in a sample. For example, a maternal sample containing both fetal and maternal DNA may contain three different alleles for a particular chromosomal region of interest. As discussed above, these three alleles would be variant sequences of the chromosomal region of interest. Methods known in the art and described further herein can be used to determine how many molecules of each of the three different alleles are present in the sample. In such an exemplary embodiment, comparing the three alleles would include comparing the number of molecules of each of the three alleles. Comparing alleles in further embodiments of the invention can include without limitation evaluating the concentration of alleles in a sample and evaluating relative concentrations and/or relative numbers of molecules of alleles in a sample.

In a further embodiment, evaluating relative concentrations or numbers of molecules in a sample includes calculating an “allelic ratio” among the alleles in a sample. This allelic ratio is the ratio of the amount of molecules of each of the alleles of interest present in the sample, and may be calculated in any number of ways known in the art and described further herein. For example, if a sample comprises two alleles A and B, and a standard method in the art (such as sequencing) shows that there are twice as many molecules of B as there are of A, then one embodiment of an allelic ratio may be the characterization of A:B as 1:2. In further embodiments, such as those containing a calculation of multiple alleles, the allelic ratio may be a standard ratio—for example, for three alleles A, B, and C, the allelic ratio may be expressed as the number of molecules of A:B:C. In further embodiments, a more complex relationship may be described as an allelic ratio, such as (A−B)/C. Such embodiments are discussed in further detail herein.

Methods for detecting and quantifying alleles or any other sequence of interest are known in the art. Such methods include without limitation any methods that detect DNA (including without limitation genomic and cDNA) and RNA (including without limitation mRNA, microRNA, and silent RNA). Such methods for detecting nucleic acids can include without limitation sequencing methods, gel electrophoresis, mass spectrometry, detection of methylation patterns, PCR methods, high performance liquid chromatography (HPLC) and the like. Methods for detecting and quantifying alleles in accordance with the present invention provide the sequence of the alleles present in the sample (or identify the presence of an allele of interest) and may also provide the number of molecules of each of the alleles in the sample. Methods for detecting and/or quantifying alleles that are of use in the present invention also include methods that quantify the relative amounts of two or more alleles of interest in a sample. By “relative amounts” is meant relative numbers of molecules and/or relative concentrations.

Sequencing methods of use for detecting and quantifying alleles of interest according to the present invention include without limitation single molecule sequencing, sequencing by synthesis, sequencing using arrays (hybridization and/or ligation), capillary sequencers, Sanger sequencing, constant denaturant capillary electrophoresis (CDCE), cycling temperature capillary electrophoresis (CTCE), polony sequencing, pyrosequencing, shot-gun sequencing, and the like. Commercial high throughput sequencing platforms for detecting and quantifying alleles of interest and such platforms are known in the art and can include without limitation: Illumina's GA, Life Technologies' SOLiD, Roche's 454, Pacific Biosciences single molecule sequencing platform, Oxford Nanopore, Ion Torrent, Complete Genomics, Nimblegen, Helicos Biosciences, Lingvitae, Nabsys, and Visigen Biotechnologies.

PCR methods of use for detecting and quantifying alleles of interest according to the present invention include without limitation digital PCR and competitive PCR.

Methods such as those described above and known in the art can be used to compare alleles in a sample by providing information on the identity (i.e., the sequence) of the alleles as well as the number of molecules of each allele present in the sample.

In general, methods of the invention include detecting alleles of a specific chromosomal region. As has been discussed above, the term “chromosomal region” refers to all or part of a chromosome. Detection of such alleles can be conducted using any method known in the art, including the sequencing and PCR methods described above. In some embodiments, the sample being analyzed is from a pregnant female and contains both maternal and fetal DNA. Detecting and/or quantifying the number of different alleles in the sample and the number of molecules of those alleles that are present in the sample can identify the presence of a chromosomal abnormality in a fetus.

In a further exemplary embodiment, a sample contains three alleles of a chromosomal region that is being analyzed. Two of the alleles are from a known source, while the third allele is expected to come from a separate source. Quantification of the number of molecules present in the sample of each allele allows comparison between the alleles from the known source and the third allele.

In a still further exemplary embodiment, the sample is a maternal sample containing both fetal and maternal nucleic acids. In this example, the sample again contains three alleles of a particular chromosomal region. If the maternal genome is heterozygous for this allele, then two of the three alleles detected in the sample are from the maternal germline DNA, while the third allele is expected from the paternal DNA contribution (if the paternal DNA contains an allele not present in the maternal genome). Quantification of the three alleles therefore allows comparisons between the two maternal alleles from the mother, allowing the determination of which allele and how many molecules were contributed by the mother to the fetus. Quantifying the number of molecules of the alleles also provides information on how many molecules of the third allele were contributed by the father to the fetus. Comparison of the maternally inherited and paternally inherited alleles further allows a quantification of the maternal and paternal contributions to the fetal DNA.

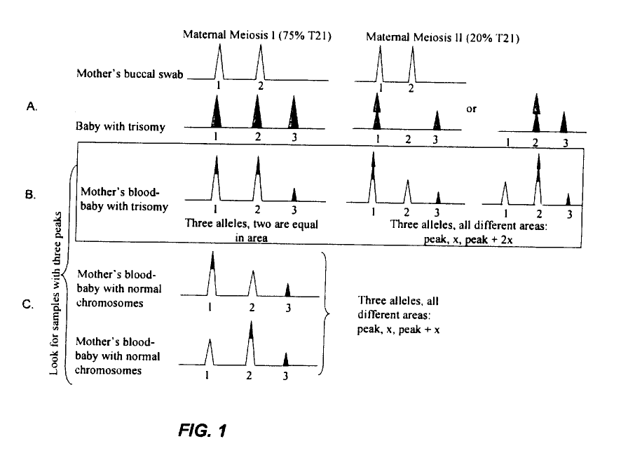

FIG. 1 is a schematic representation of the comparisons that can be made between different alleles detected in a sample. In FIG. 1, a maternal sample in which three alleles are detected is shown. The peaks illustrated in FIG. 1 represent the number of molecules of each allele present in the sample. As discussed herein, these “peaks” (i.e., the number of molecules) can be determined using methods well known in the art and described herein. For example, the area under the peaks from an electropherogram from a CDCE analysis of a sample provides information on the number of molecules of each detected allele. Similarly, the output of a sequencing platform will provide a count of the number of molecules for each allele, which can also be depicted by the peaks in FIG. 1.

In an exemplary embodiment, genetic abnormalities, including chromosomal abnormalities, are detected by comparing the number of molecules of alleles in a sample. As is generally illustrated in FIG. 1, a sample comprising both fetal and maternal nucleic acids will be expected to show specific relationships between the number of molecules for alleles of a particular chromosome. For example, if the maternal genome is heterozygous for a particular chromosomal region, then two alleles detected in the sample will be the maternal contribution. Since the sample contains both fetal and maternal DNA, the number of molecules detected for those two alleles will include the numbers of molecules from the maternal DNA and the numbers of molecules for the maternally-inherited allele in the fetal DNA (also referred to herein as the “maternally-inherited fetal allele”). The fetal genome will contain maternally and paternally-inherited alleles. If the paternally-inherited allele in the fetal DNA (also referred to herein as the “paternally-inherited fetal allele”) is not present in the maternal genome, then three different alleles will be detected in the sample containing both maternal and fetal DNA (the two alleles from the mother and the one paternally-inherited allele in the fetus). Comparing the number of molecules for each allele can then be used to detect a chromosomal abnormality.

In further embodiments, the present invention provides methods for detecting genetic abnormalities, including chromosomal abnormalities, in a fetus by detecting alleles for a location of interest on a chromosome (also referred to herein as a “genetic location”), where the maternal genome is heterozygous at that location of interest and the fetus inherits a different allele from the father at that same genetic location. In still further embodiments, methods of the invention detect a paternally-inherited fetal allele in a sample comprising both maternal and fetal nucleic acids, where that paternally-inherited fetal allele is not present in the maternal genome. Detection of such a paternally-inherited fetal allele will in such embodiments indicate that the fetus does have a genetic abnormality.

As will be appreciated, the term “maternal allele” may refer to the allele inherited by the fetus from the mother and/or the alleles in the maternal genome. In a sample comprising both fetal and maternal DNA, the molecules of “maternal allele” detected and quantified will include both molecules of the allele from the maternal DNA as well as molecules from the fetal DNA of the maternally-inherited fetal allele.

For example, in approximately 95% of trisomy cases, the fetus inherits two copies of the chromosome from the mother. In this situation, three alleles of that chromosome or a particular region of that chromosome are detected in the sample (represented by the three peaks in FIG. 1). As discussed above, if the mother is heterozygous, two of the alleles detected are the maternal alleles. The number of molecules detected for those two alleles will reflect the molecules from the maternal DNA in the sample plus the molecules of those same two alleles from the fetal DNA (i.e., the maternal alleles plus the maternally-inherited fetal alleles). The third allele detected will be the fetal allele that is paternally inherited and is not present in the maternal genome. In this example, trisomy is detected if the number of molecules for the two maternal alleles is equal (first trace of FIG. 1B) or if all three alleles are present in different numbers in the relationship of peak, x and peak+2x, where “peak” represents the number of molecules for one of the maternal alleles, “x” represents the number of molecules for the paternally-inherited fetal allele, and “peak+2x” is the number of molecules for the other maternal allele (right two traces of FIG. 1B). In the situation in which the two maternal alleles have equal numbers of molecules, trisomy is detected, because the numbers for the two maternal alleles are actually a sum of the maternal alleles and the maternally-inherited fetal alleles. The only way for the numbers for both maternal alleles to be equal in a sample comprising both fetal and maternal DNA is if the fetus inherited two alleles from the mother. The presence of the paternally inherited allele indicates that the fetus must then have trisomy, because it has both alleles from the mother and one from the father.

Similarly, in the situation in which all three alleles are present in the sample in the relationship of peak, x and peak+2x, this also indicates that the fetus has trisomy. The “peak” represents the number of molecules of maternal allele. “x” represents the number of molecules of the paternally-inherited fetal allele. “x” therefore represents a kind of internal standard of how much fetal DNA is present in the sample. The allele with the number of alleles “peak+2x” thus contains alleles from the mother as well as two copies of the allele from the fetus. These two copies are the maternally-inherited fetal DNA. Therefore, the fetus again has inherited two alleles from the mother and one from the father, which is an indication of trisomy.

The same analysis of the number and identity of the alleles in a sample can also be used to determine that the fetus does not have trisomy. As discussed above, this analysis must be conducted for an allele for which the maternal genome is heterozygous. If three alleles are detected in a sample containing both maternal and fetal DNA, then two of the alleles will be from the heterozygous mother, and the third allele is the paternally-inherited fetal allele. For a fetus without trisomy, the number of molecules of the three alleles will all be different, but unlike the case with trisomy above, the number of molecules of the three alleles will be in the relationship of “peak”, “x” and “peak+x”. “Peak” represents the number of molecules of one of the maternal alleles. “x” represents the number of molecules of the paternally-inherited fetal allele. “Peak+x” represents the number of molecules of the second maternal allele—this number includes both the number of molecules of the allele from the maternal DNA and the number of molecules of the maternally-inherited fetal allele. Thus, if the fetus has inherited only one chromosome from each parent, there should be a larger number of molecules for the allele the fetus inherited from the mother and a smaller number of molecules for the allele the fetus inherited from the father. Since the sample comprises maternal and fetal DNA but no paternal DNA, the only molecules of paternal alleles present in the sample are molecules of the paternally-inherited fetal allele. As a result, the number of molecules of the paternally-inherited fetal allele can provide an internal standard of how much fetal DNA is present in the sample relative to the maternal DNA.

In further embodiments, analysis of the number of molecules of alleles in a sample provides a measure of “allelic dosage”. By “allelic dosage” as used herein is meant the number or concentration (or relative number or concentration) of molecules of each allele that is present in a sample. Thus, calculation of an “allelic ratio” as discussed above provides a mechanism to measure the dosage of each allele. Furthermore, in an analysis of a sample comprising both fetal and maternal nucleic acids, determining the number of molecules of paternally-inherited fetal alleles also provides a measure of the paternally-inherited fetal allelic dosage. This paternally-inherited fetal allelic dosage can serve as an internal standard that can be used to determine the maternally-inherited fetal allelic dosage. Thus, a calculation of allelic dosage provides a measure of the allelic dosage from each parent to the fetus. Calculations of allelic dosage can be used to detect a genetic abnormality using any of the methods described herein, because the relationships expected for maternal and paternal (and maternally-inherited and paternally-inherited) are equivalent to the relationships described herein for comparing alleles.

As will be appreciated, the above methods can also be used to compare alleles based on de novo deletions, insertions, and the like. For example, in the case of 22q11.2 deletion syndrome (the most common human deletion syndrome, which is typically a 3 million basepair deletion), the deletion is a de novo event in 75% of the cases. If the de novo event is a maternally-derived deletion in the fetal genome, three alleles would again be detectable in a sample comprising both maternal and fetal DNA. However, the number of molecules of the three alleles will show a relationship in which two of the alleles are present in equal numbers and the third allele will be present in smaller numbers. In other words, two fetal alleles will not be detected in the sample, because one of those alleles would be deleted. As discussed above, for a normal fetus, all three alleles should have different numbers of molecules present in the sample, with the numbers being in the relationship of “peak”, “x” and “peak+x”. In the case of a maternally-derived deletion, the numbers would instead be in the relationship of “peak”, “peak” and “x”.

Similarly, in the case of a de novo but paternally-derived 22q11.2 deletion in the fetal genome, a third allele would not be detected in a sample containing both maternal and fetal DNA, because it would be the paternally-inherited fetal allele that would be deleted. In further embodiments, multiple assays directed to the detection of different alleles can be conducted to confirm that the lack of a third fetal allele is the result of a deletion rather than the result of a non-informative assay (i.e., an assay in which the maternal genome is not heterozygous for an allele or the paternal genome has an allele in common with the maternal genome).

In further embodiments, methods of the present invention can be used to detect alleles that are inherited. In the maternally-derived and paternally-derived deletions discussed above, the maternal and paternal genomes are normal at the deletion/insertion site, but during meiosis, deletions or insertions occur and are passed to the fetus. In contrast, deletions and insertions and point mutations (which would include basepair substitutions), small (less than 20 base pairs) deletions or insertions (also referred to herein as “small-deletions” and “small-insertions” respectively) may occur in the maternal and/or paternal genomes and are passed on to the fetus. When a target sequence is larger than the small deletion or insertion, the same comparisons described above for allelic comparisons can be used to determine allelic dosage inheritance if a neighboring SNP can be encompassed by the target sequence. For example, the delta F508 deletion in the CFTR gene is a small deletion that can be passed on to the fetus by either the mother or the father. In the case where the mother is a carrier of the delta F508 deletion, it will appear as if she has two peaks. If the father contributes a normal allele and the fetus inherits the delta F508 deletion, the maternal plasma would show two peaks of equal area and height. It would not be clear if there was no fetal DNA present in the sample, or was at levels too low to be detected. However, if a neighboring SNP is encompassed, permitting at least three possible alleles in the target sequence, it would be possible to distinguish maternally inherited from paternally inherited alleles. The term “peaks” as used herein refers to the number of molecules of an allele and to a signal associated with the number of molecules of an allele.

In one aspect, the present invention provides methods and compositions for comparing alleles, where those alleles may be comprised of haplotypes. As used herein, the term “haplotypes” refers to groups or sets of markers (including without limitation SNPs, deletions, small deletions, insertions, small insertions, methylation, and short tandem repeats) that are inherited together as a unit. Thus, alleles detected and quantified as described herein encompass multiple markers, and thus different alleles comprise different combinations of these multiple markers. As will be discussed in further detail herein, alleles of use in the present invention may comprise multiple SNPs. These SNPs may be contiguous or non-contiguous. In further embodiments, alleles for comparison may include any combination of sites, including deletion sites, insertion sites, and sites which comprise variant sequences of two or more nucleotides in length. For example, alleles detected and compared in accordance with the present invention may include a deletion site and/or an insertion site and/or one or more SNPs and/or a variant sequence comprising two or more nucleotides. Such combinations increase the number of haplotypes that can be detected for a particular genetic location, thus increasing the informative of the comparative assays of the invention, as is discussed in more detail herein.

In a further aspect, multiple alleles are detected in a single reaction or assay. Thus, by identifying one or more SNPs, methylation patterns, or any other combination of markers that can compose an allele as described above, detecting multiple alleles (e.g., detecting both maternal alleles and a paternally-inherited fetal allele) in a single reaction or assay preserves phase information on the chromosome.

II(A). Amplification of Nucleic Acids Prior to Detection

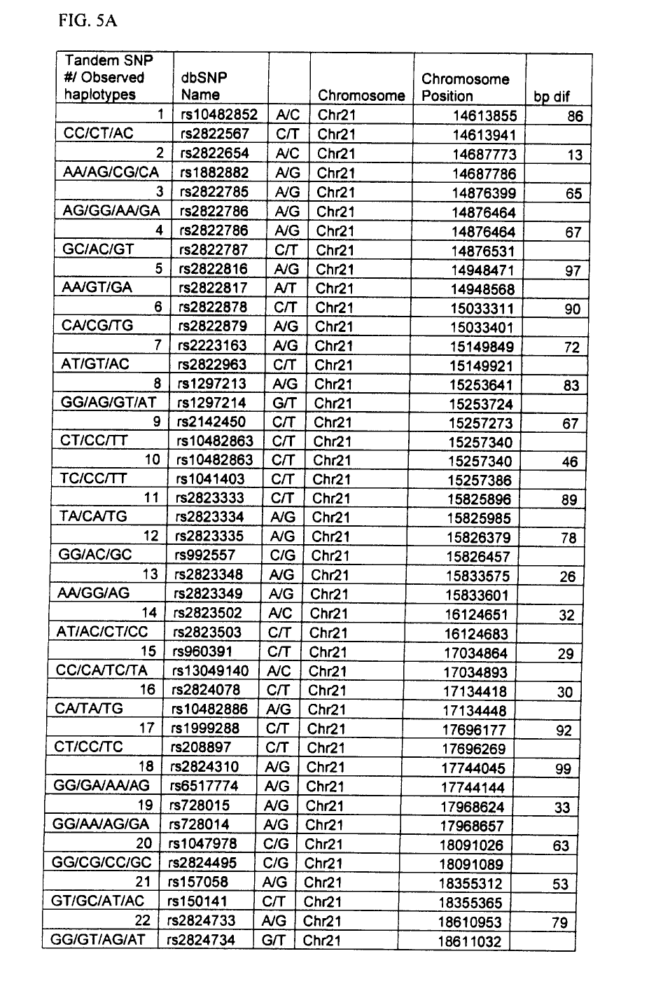

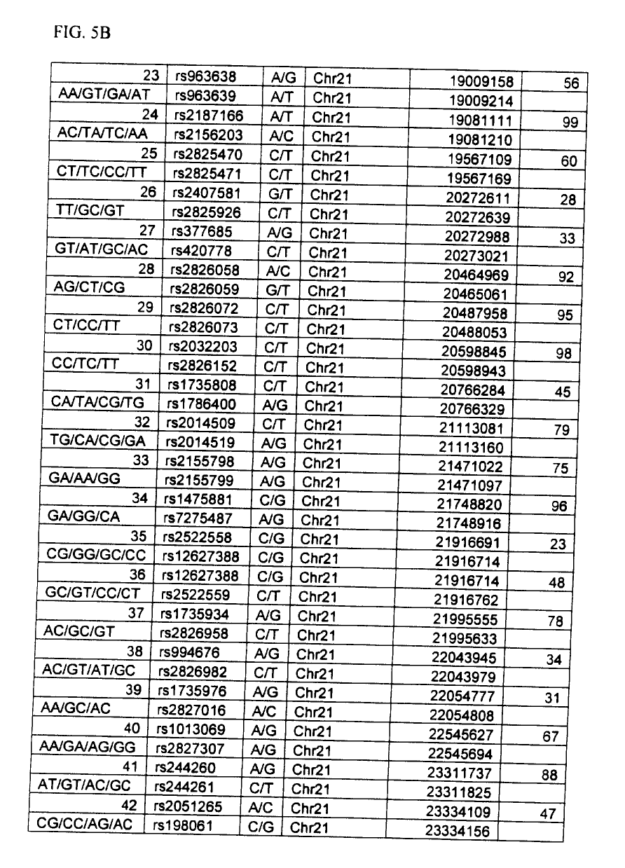

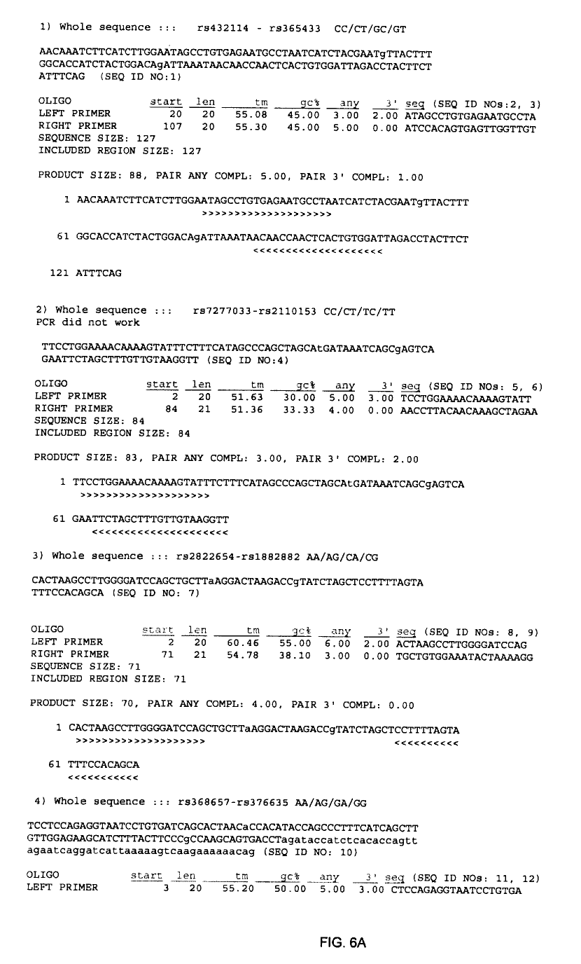

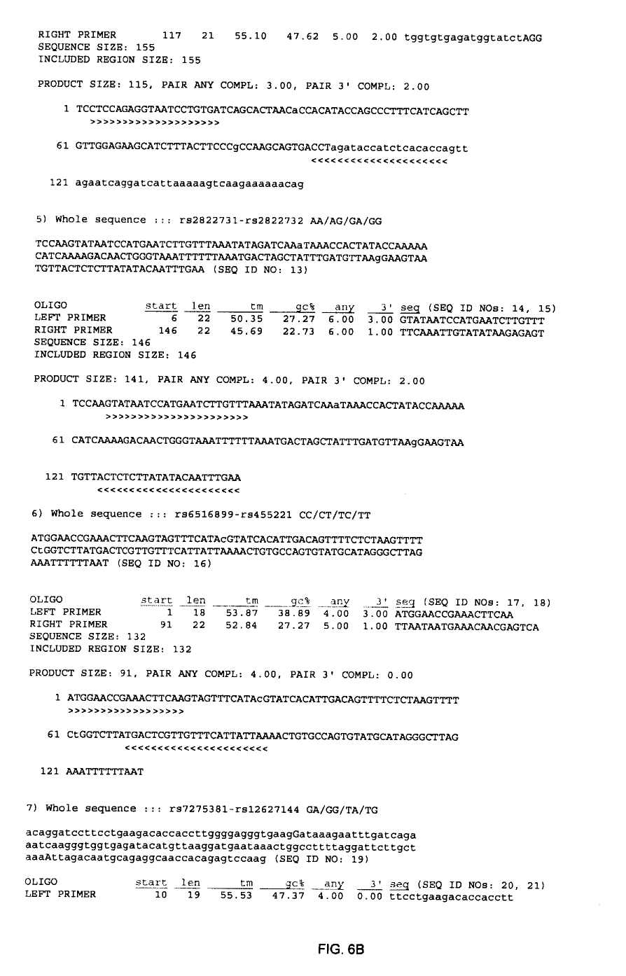

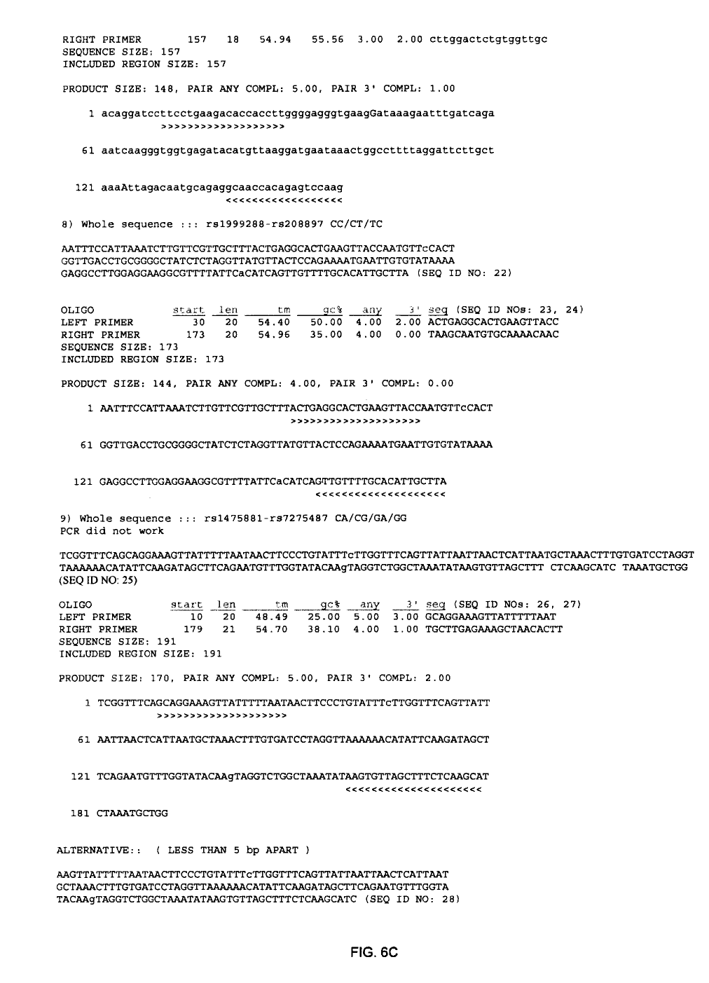

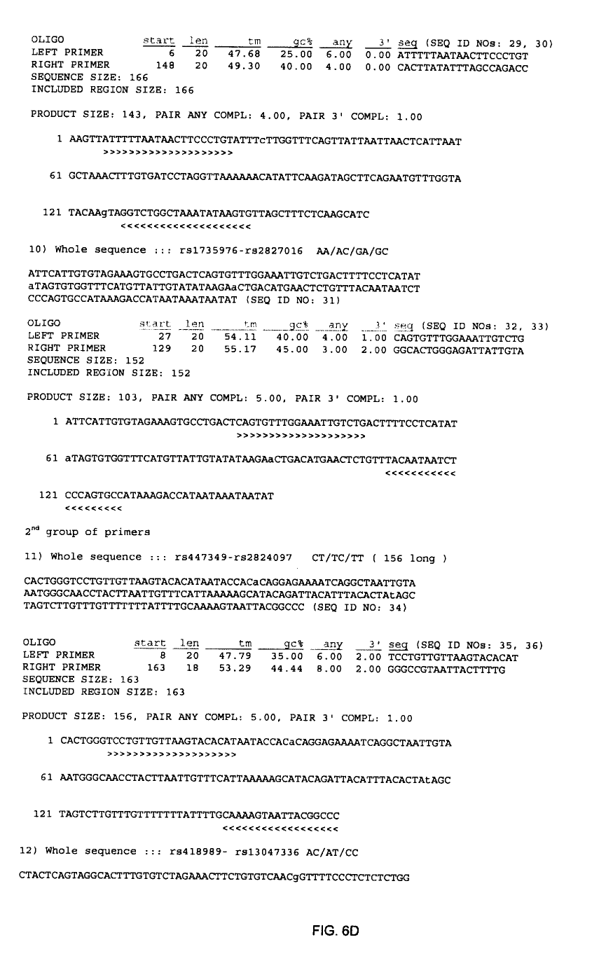

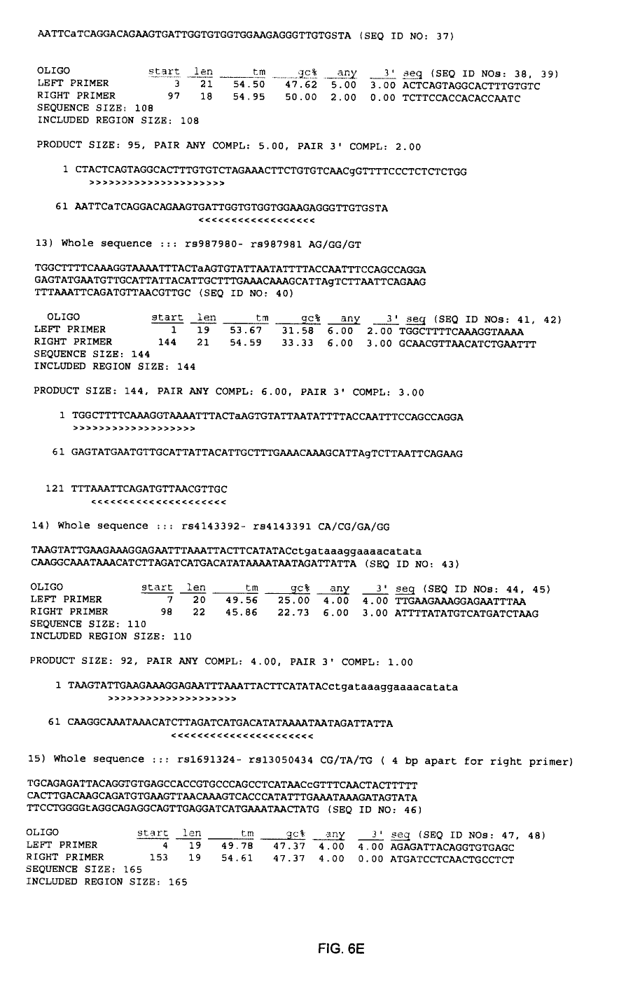

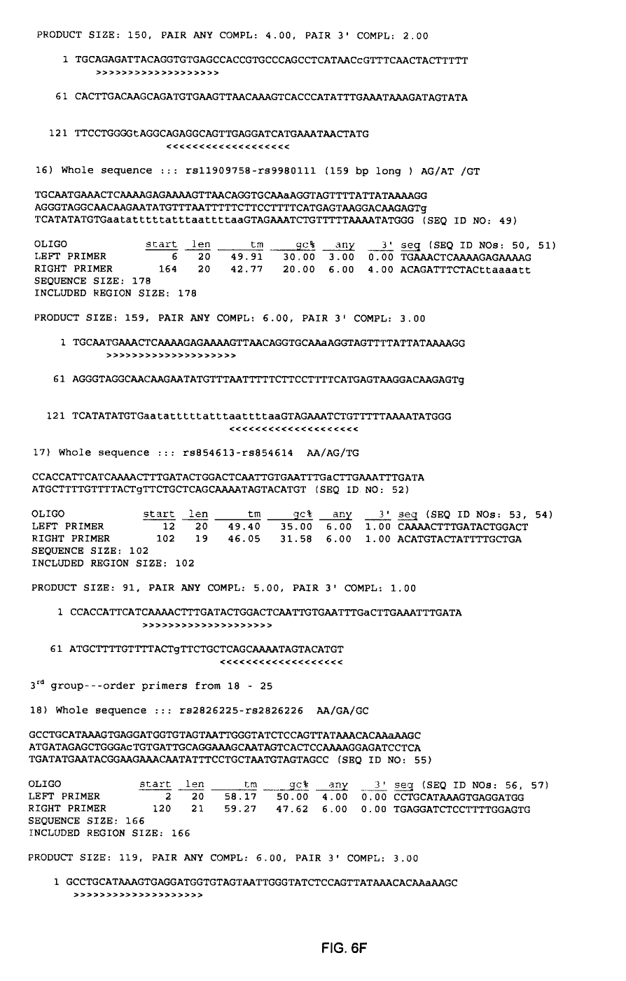

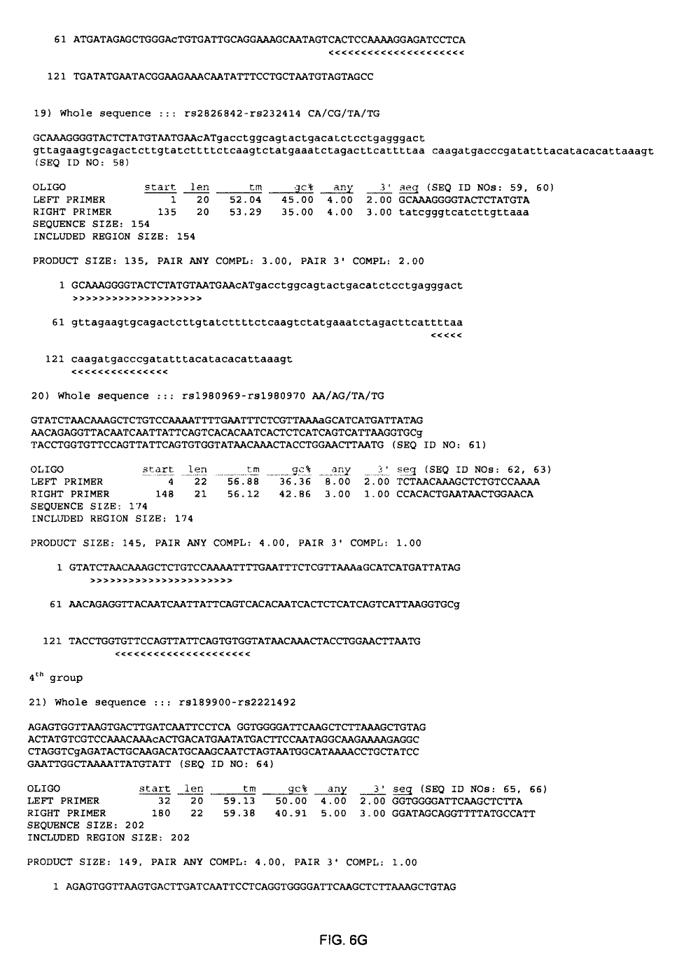

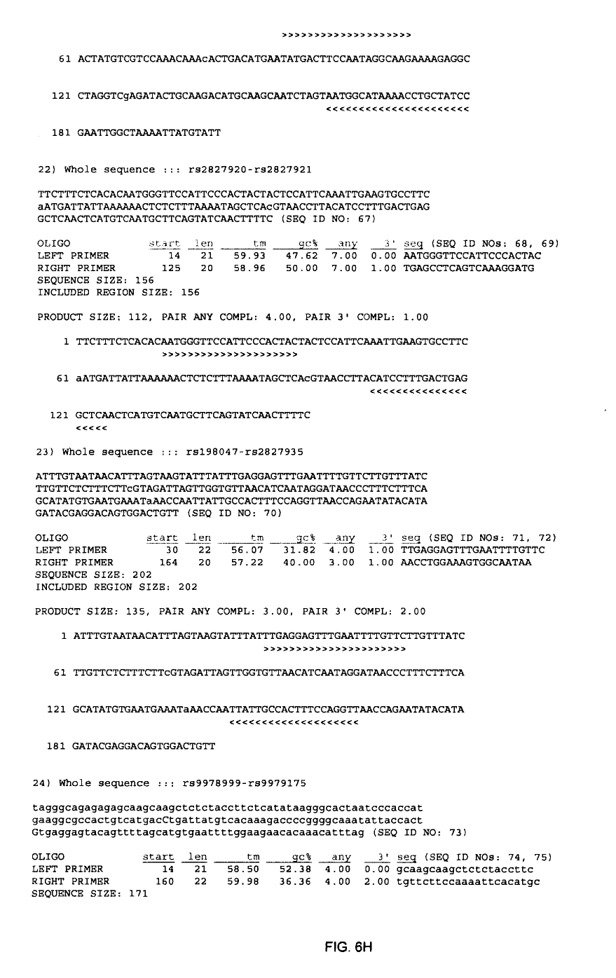

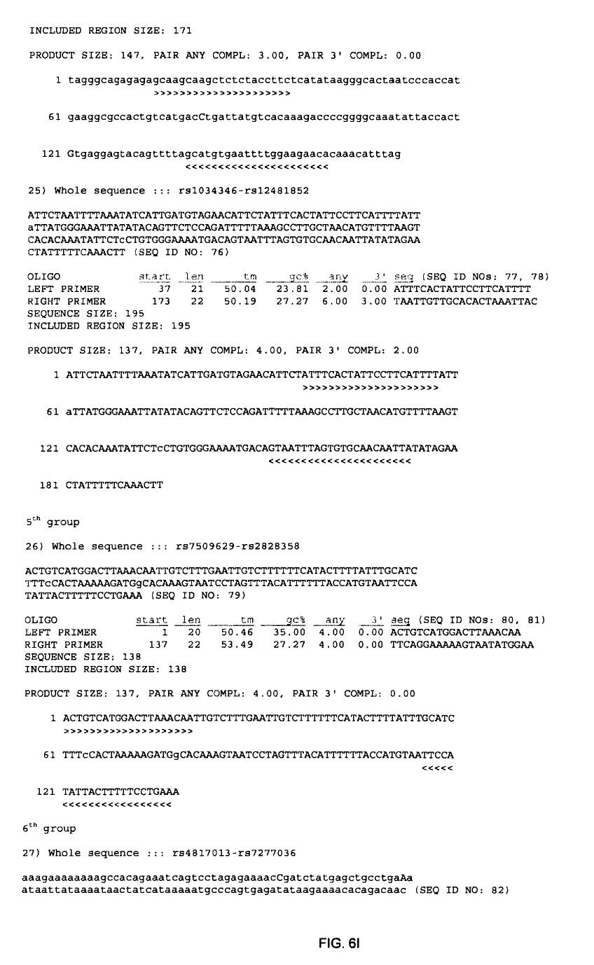

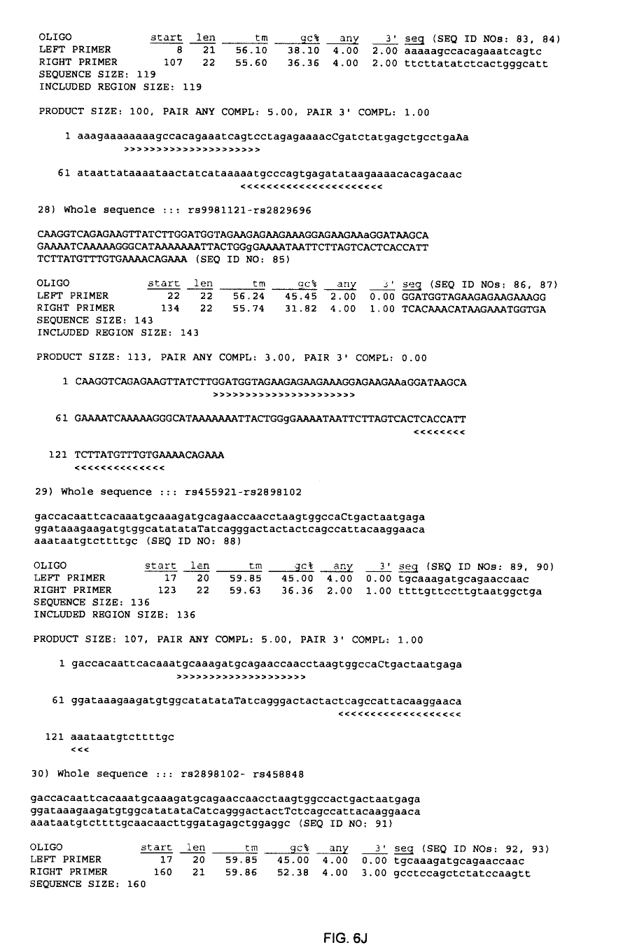

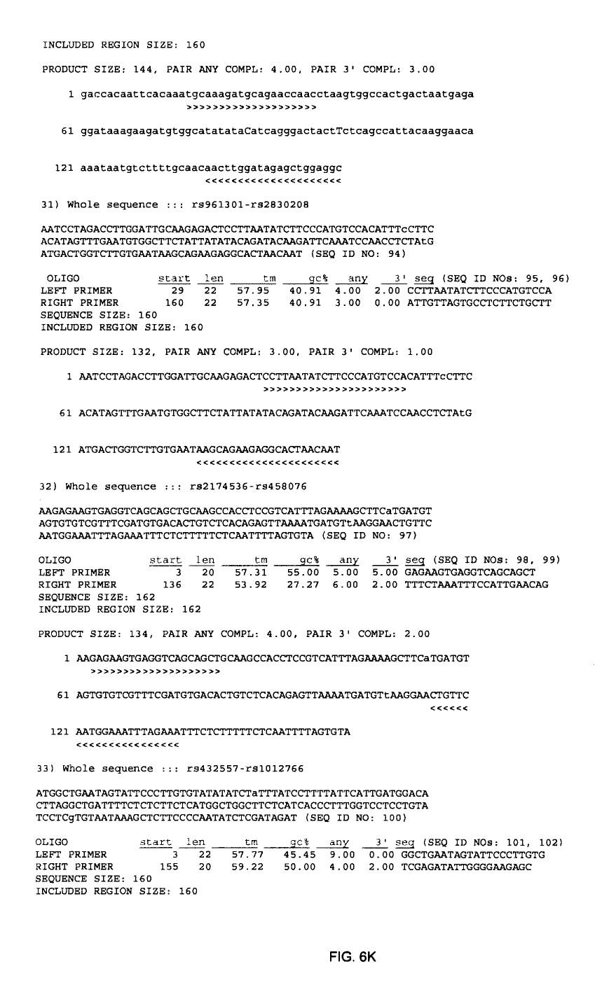

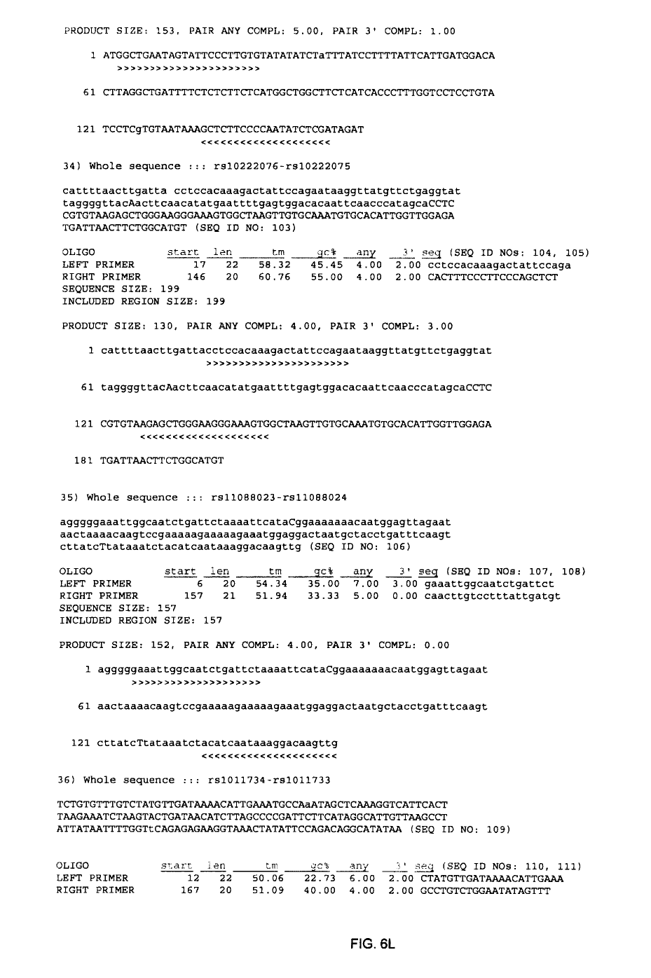

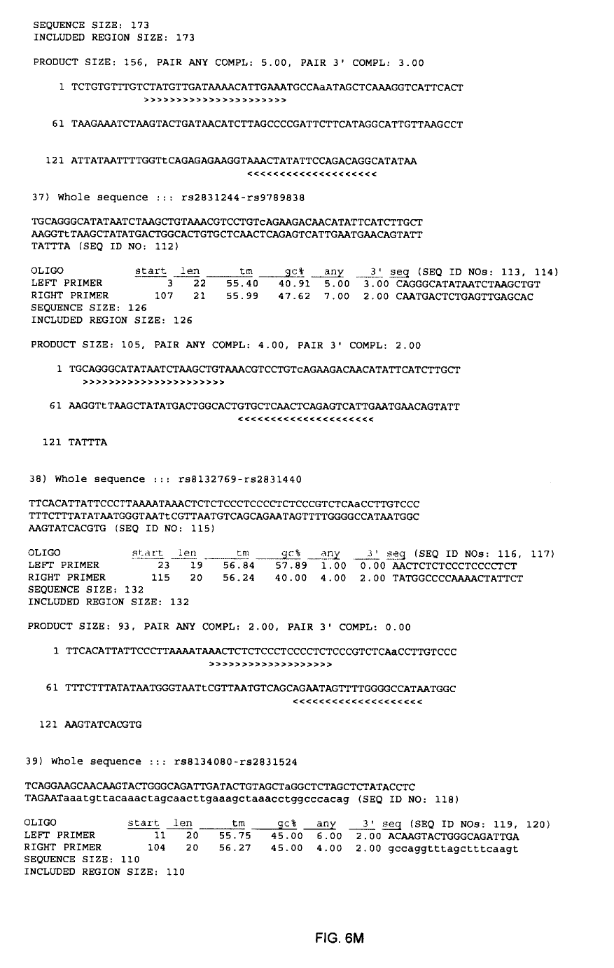

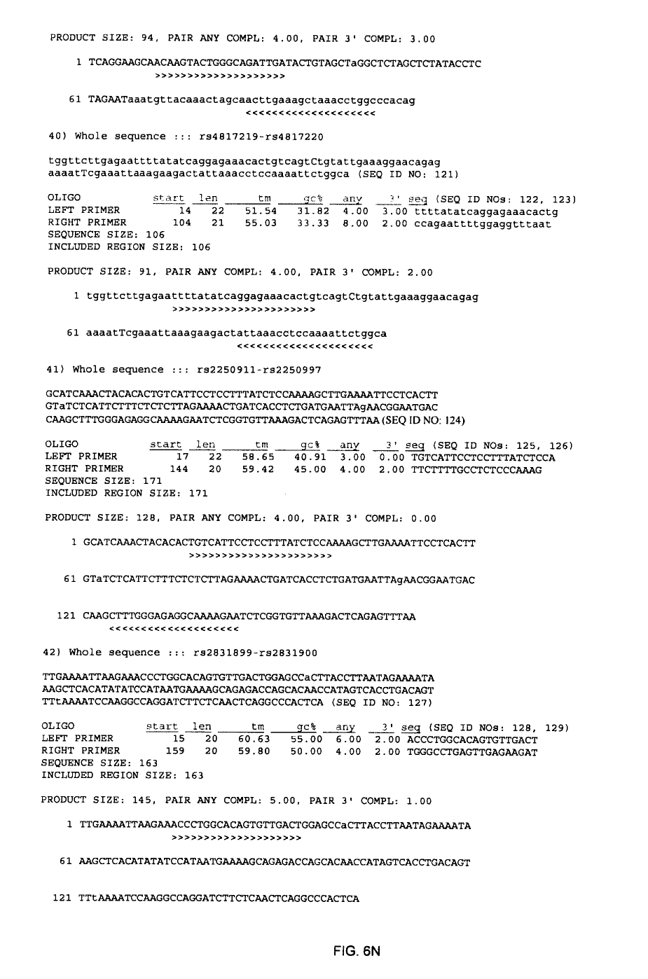

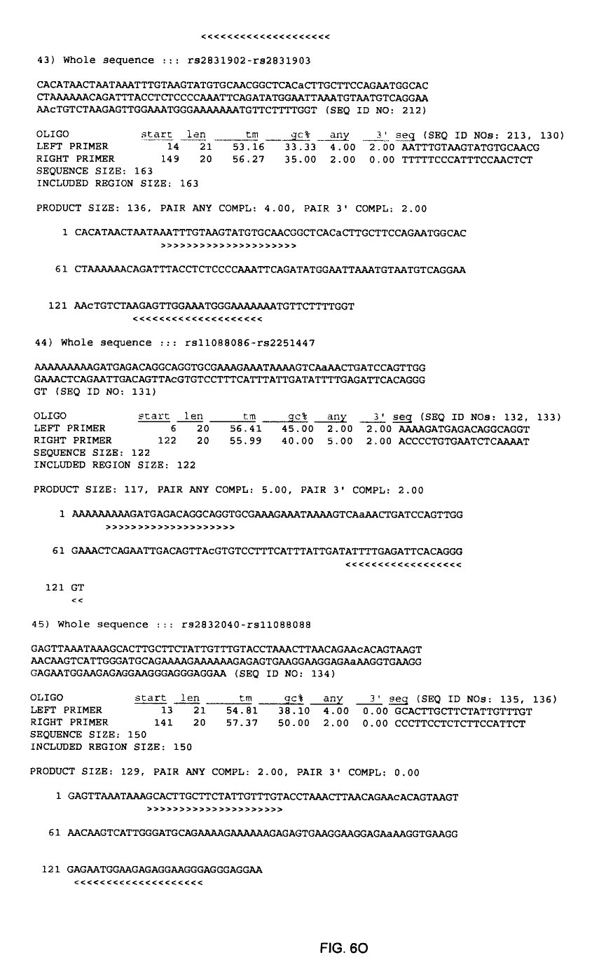

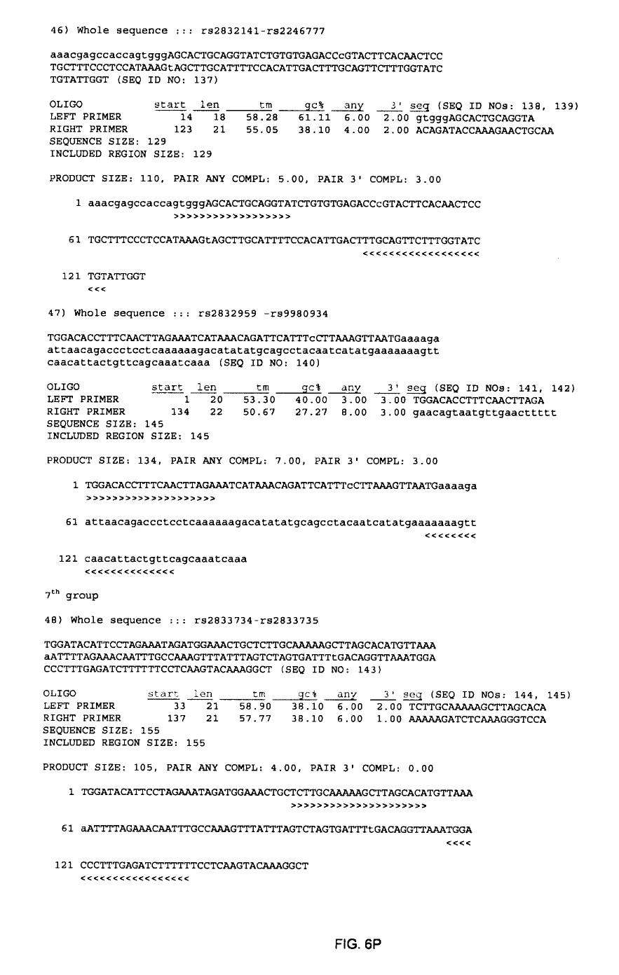

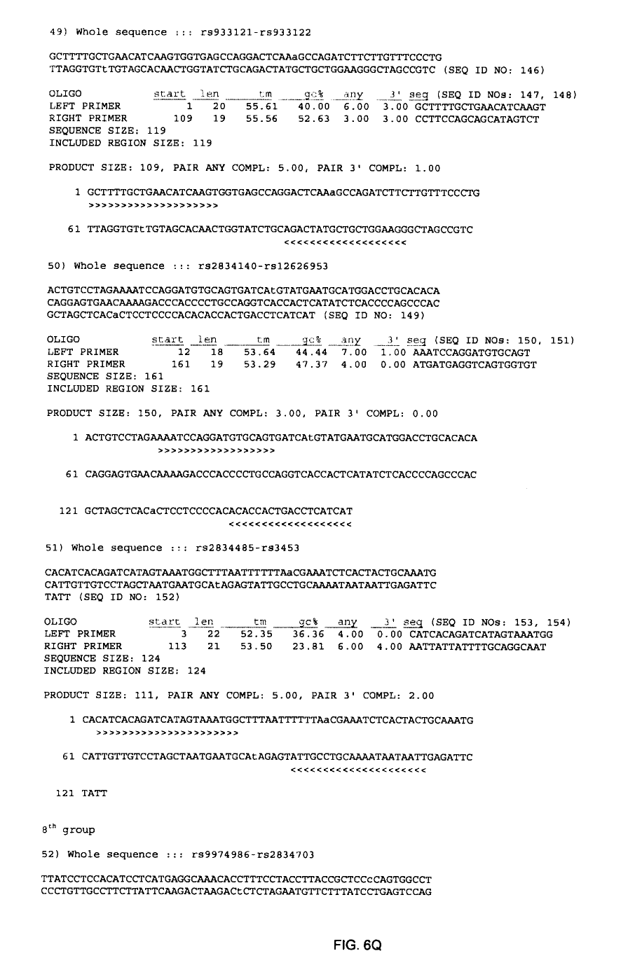

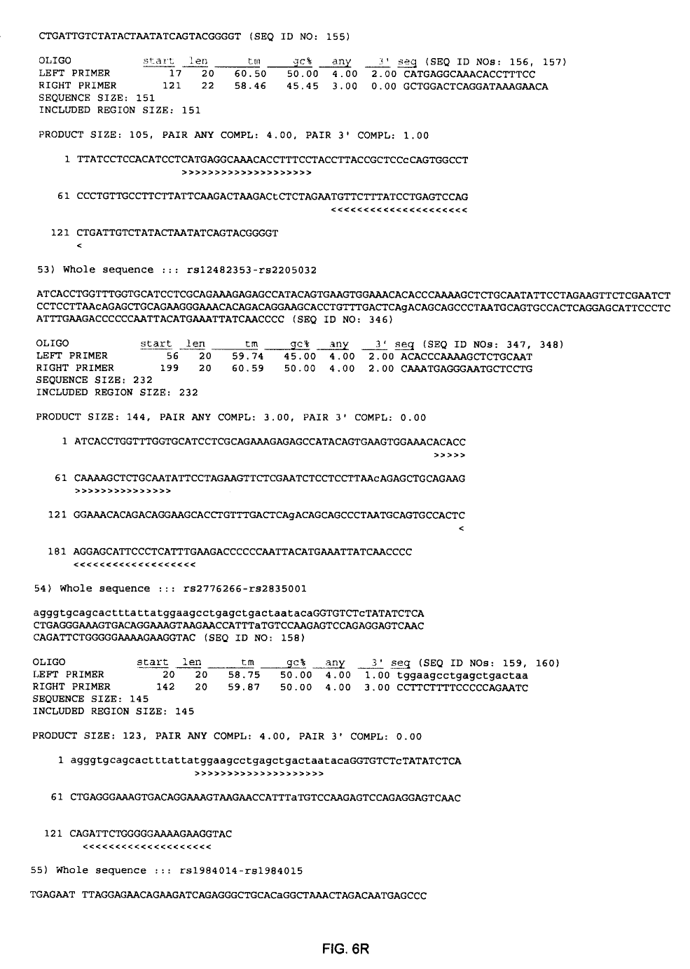

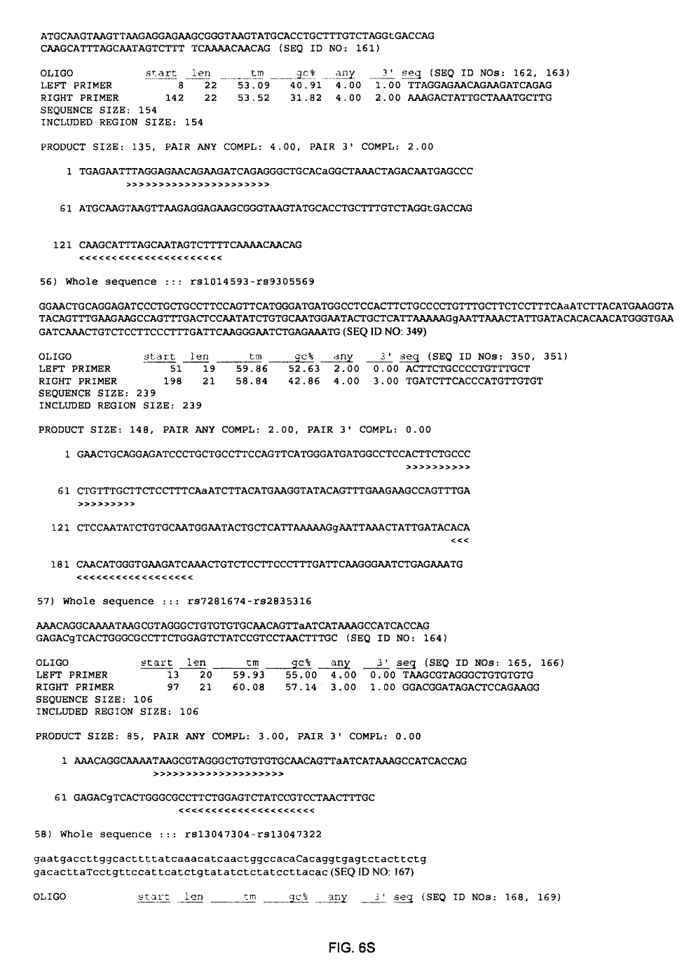

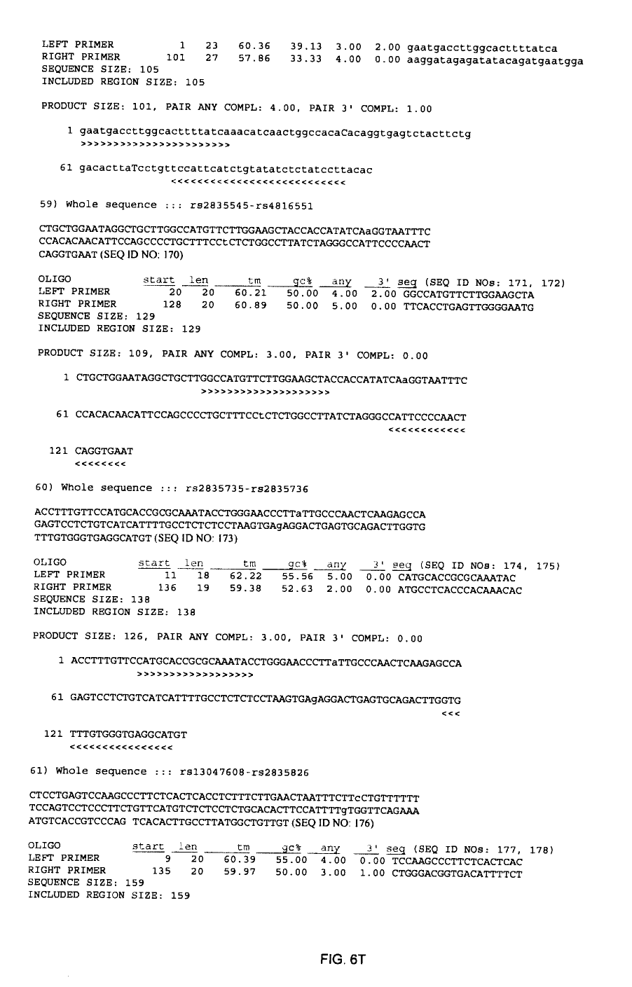

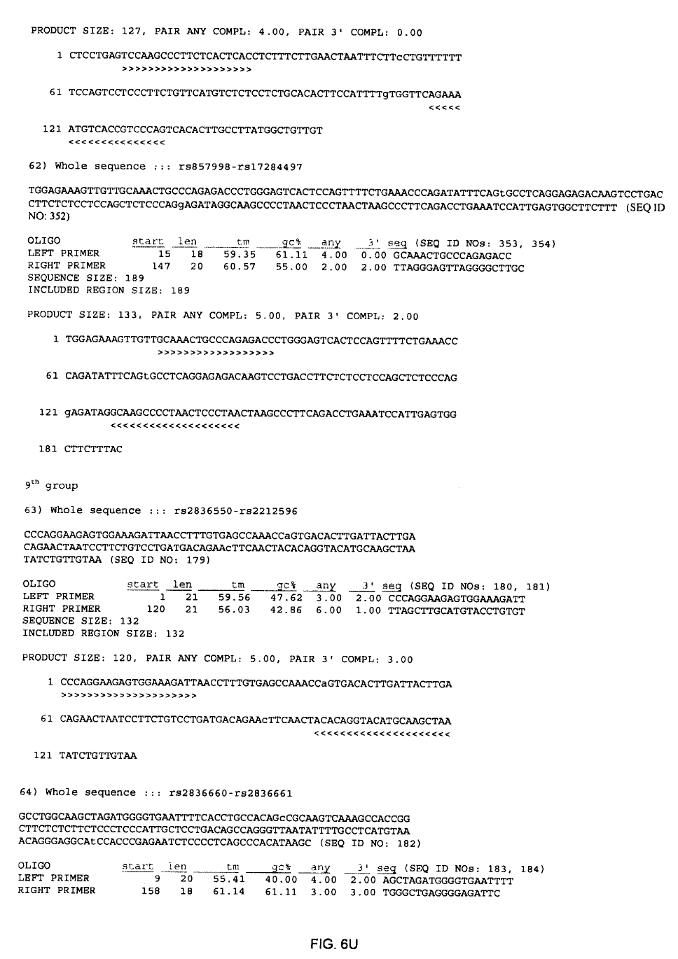

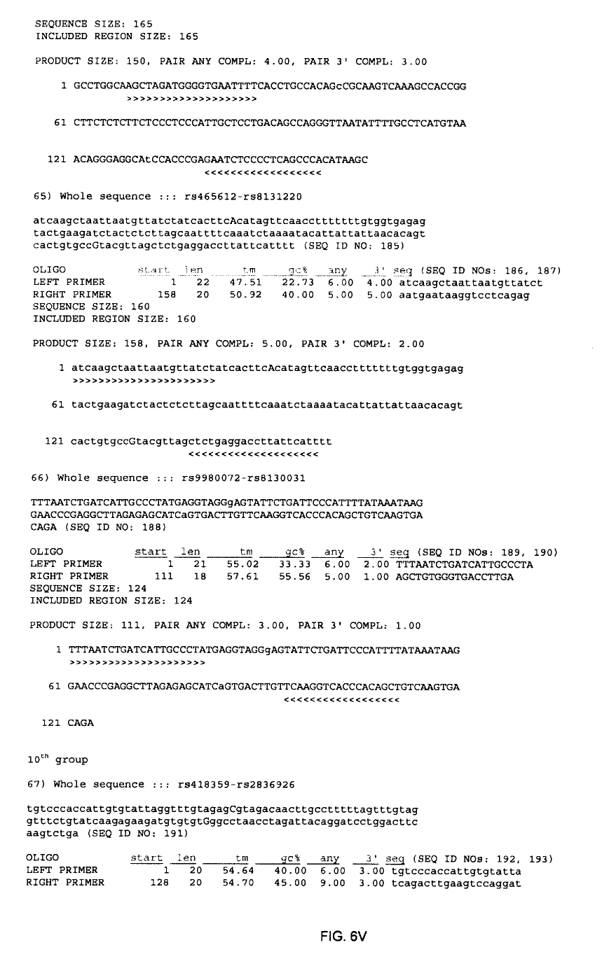

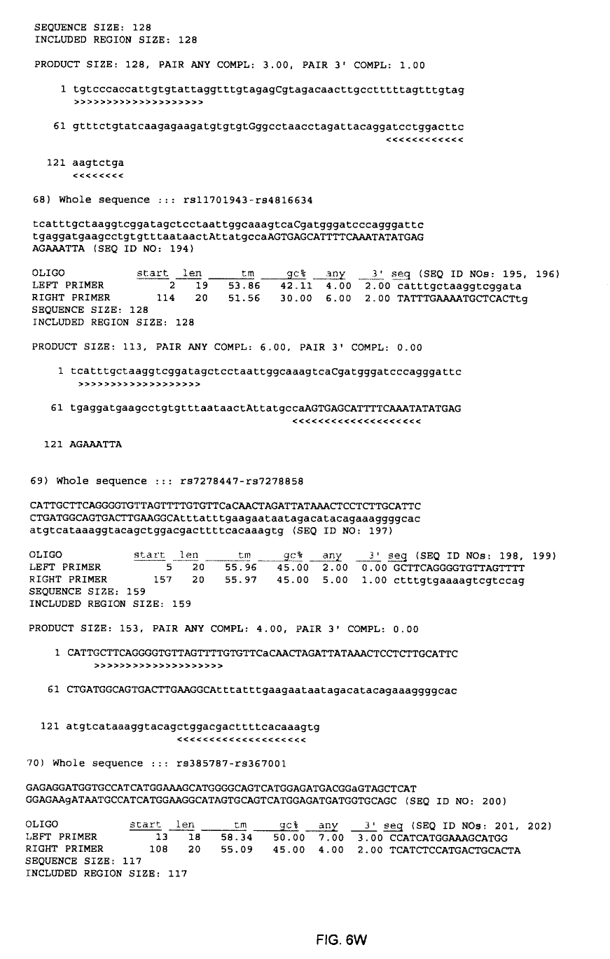

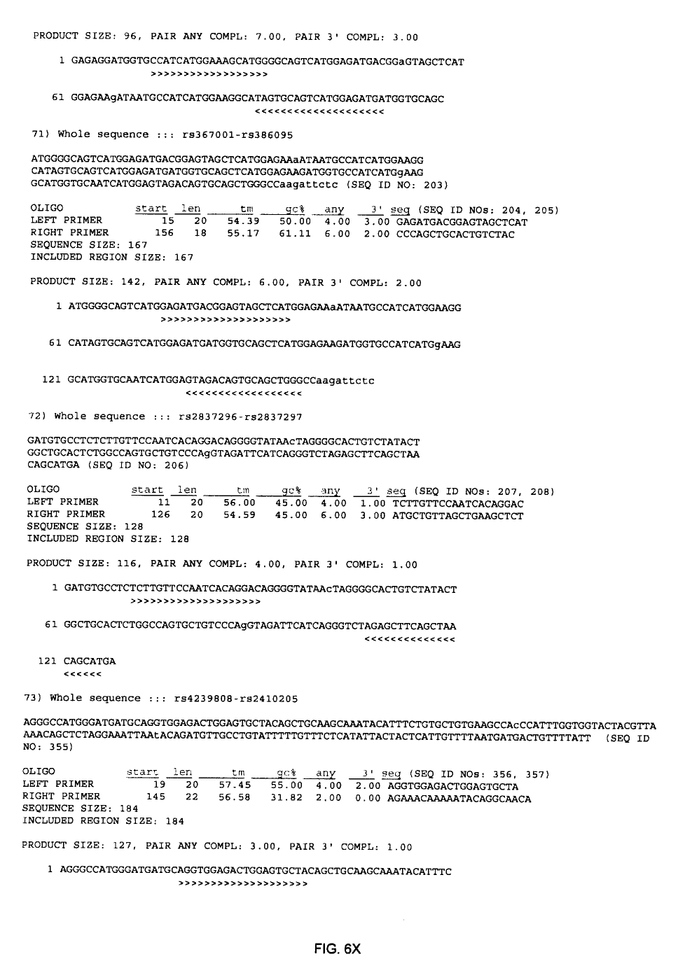

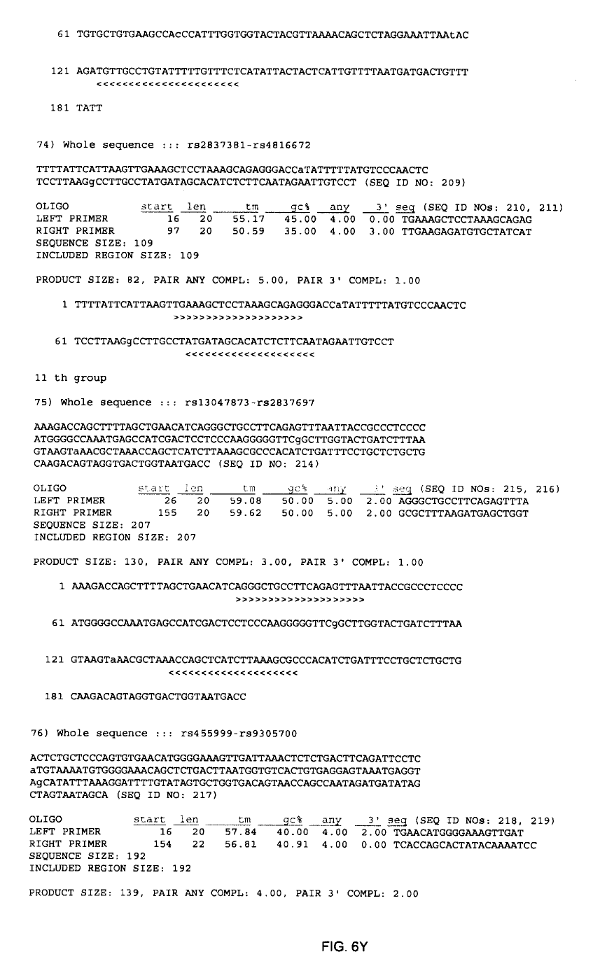

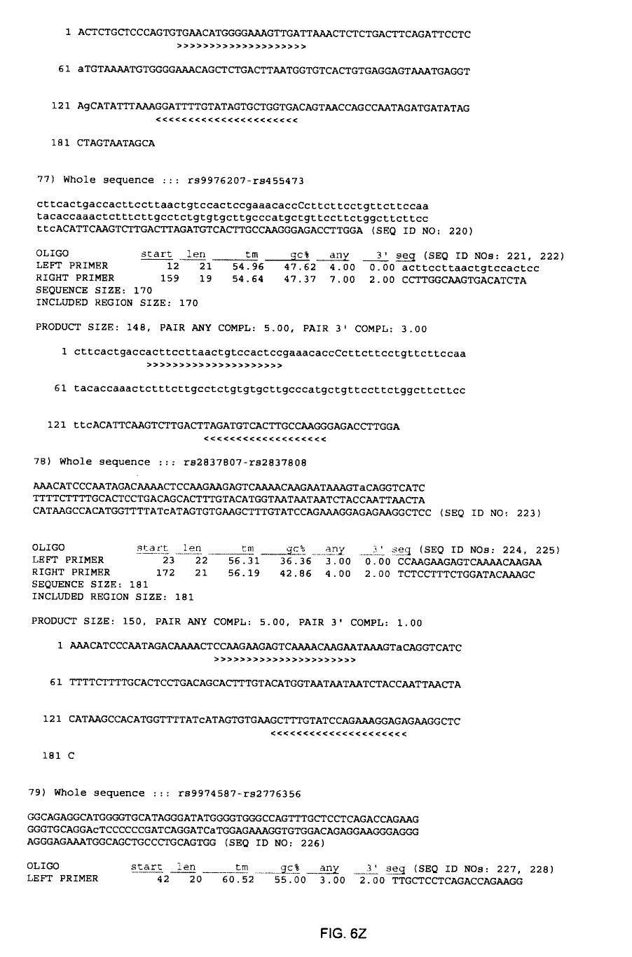

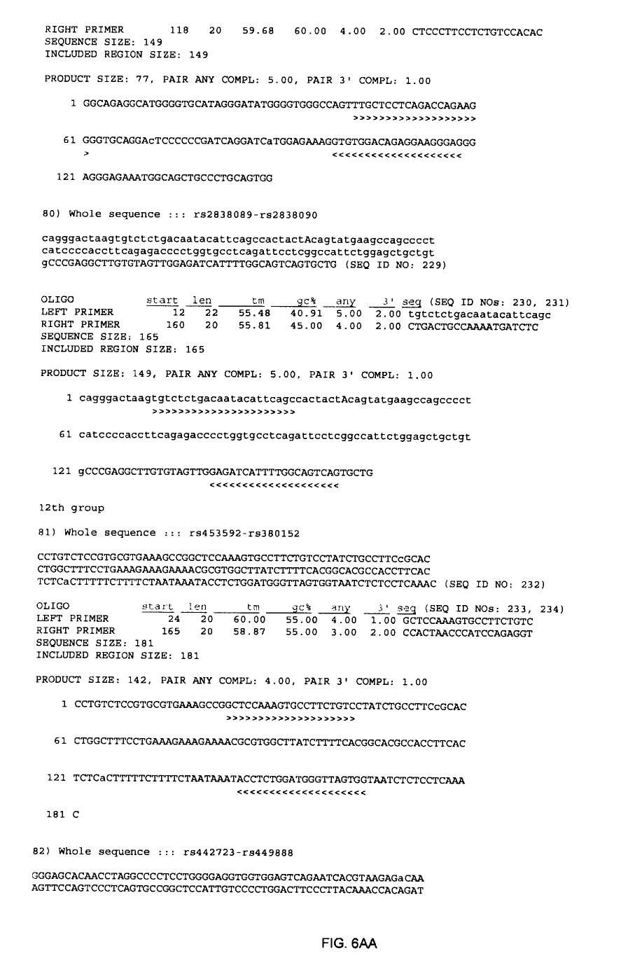

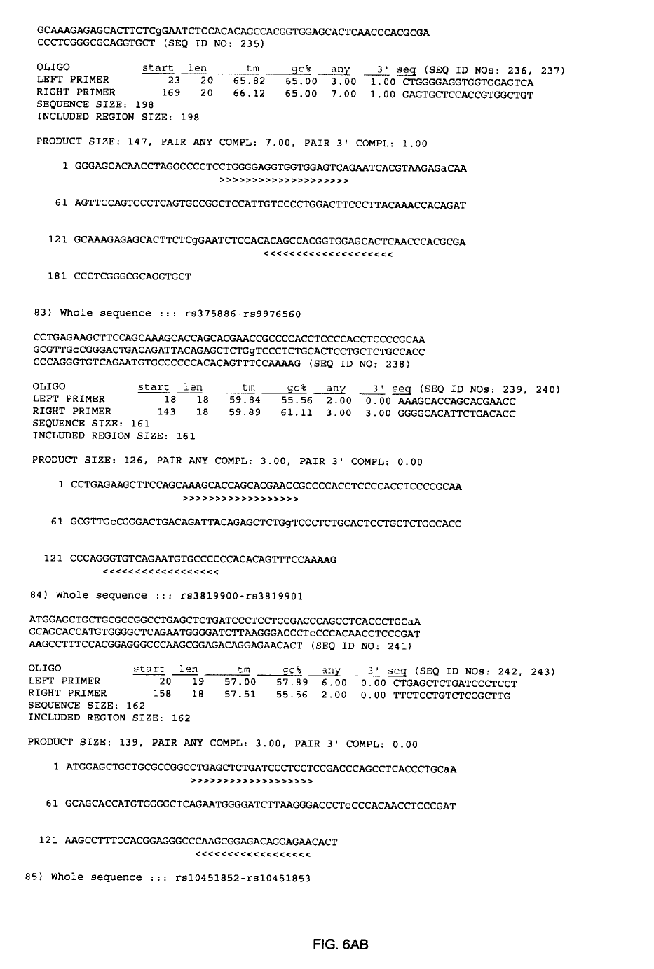

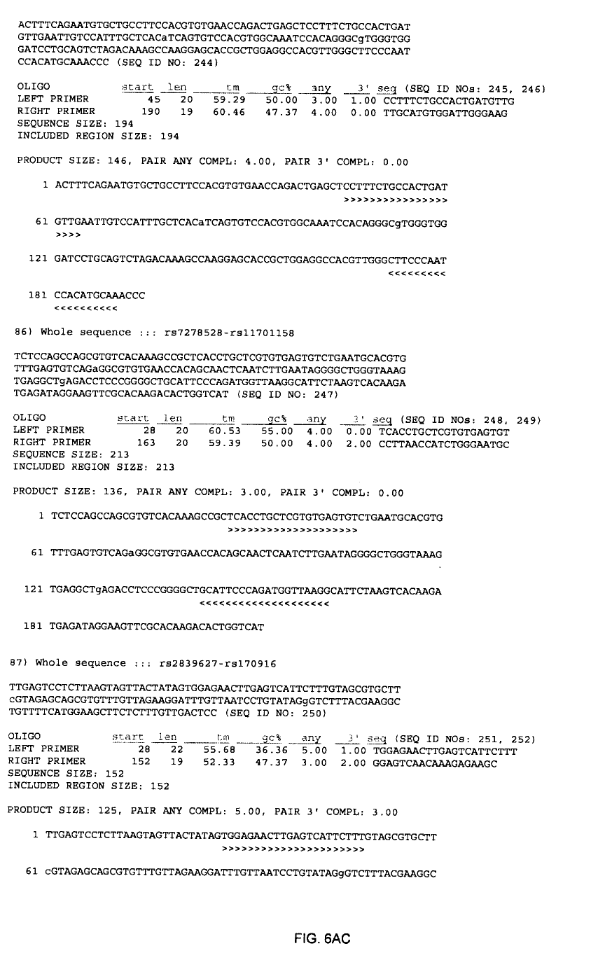

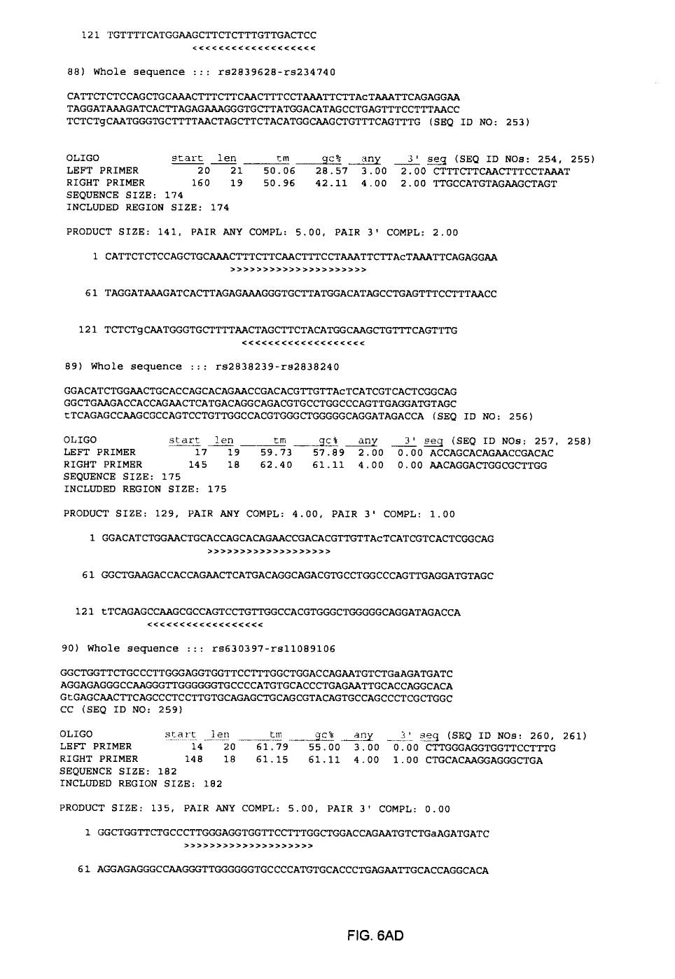

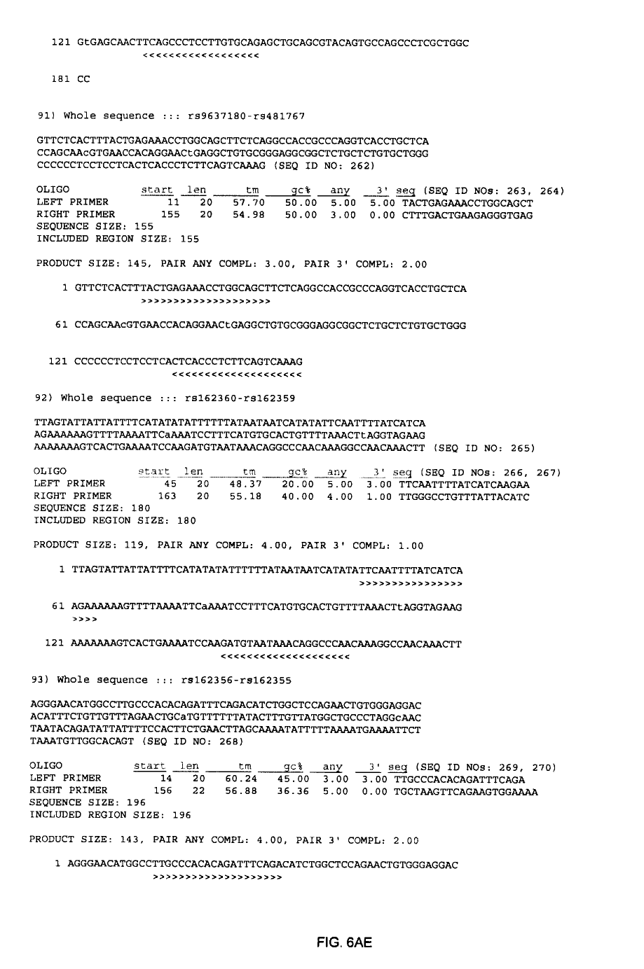

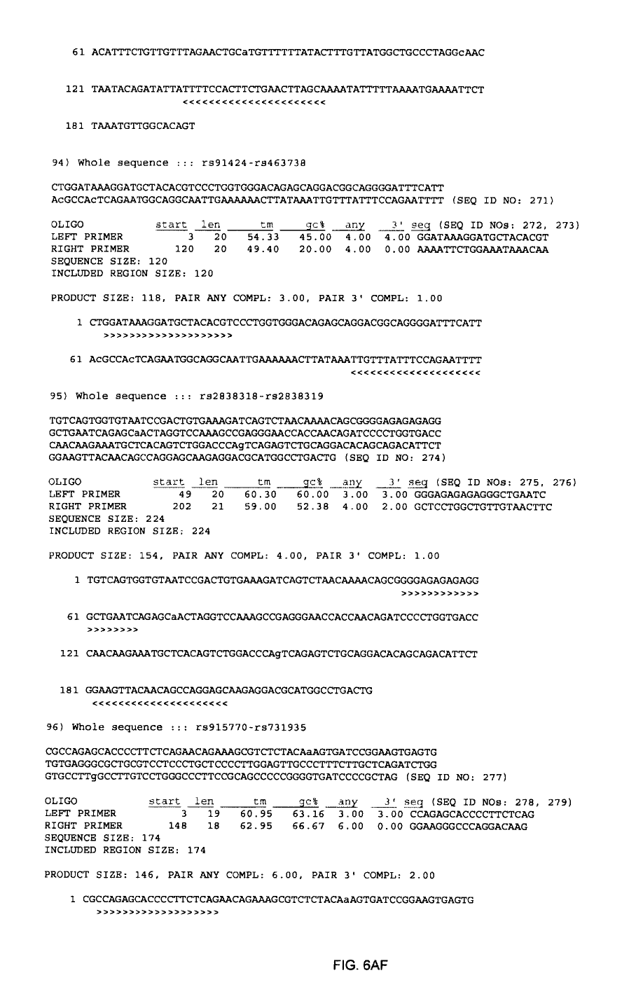

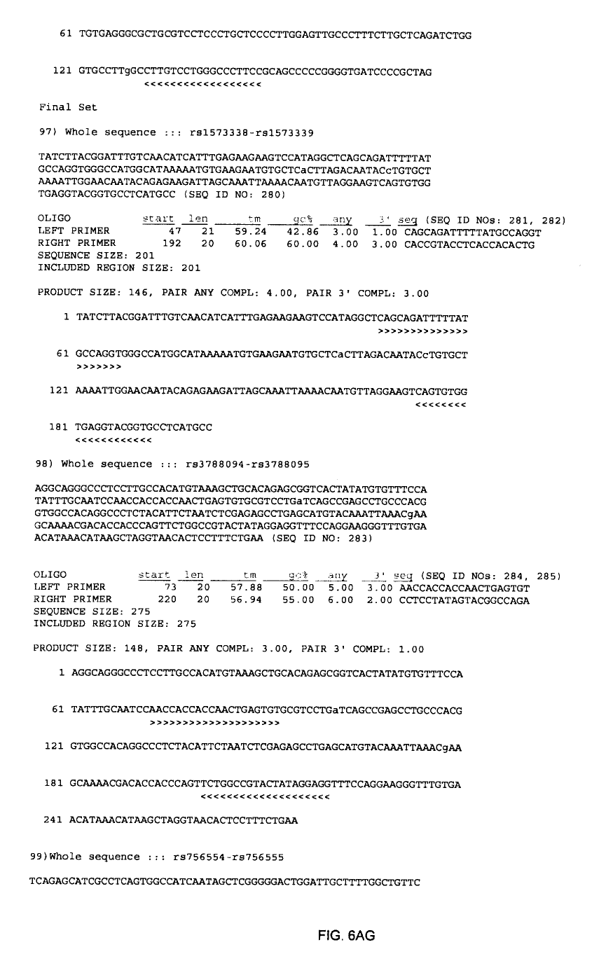

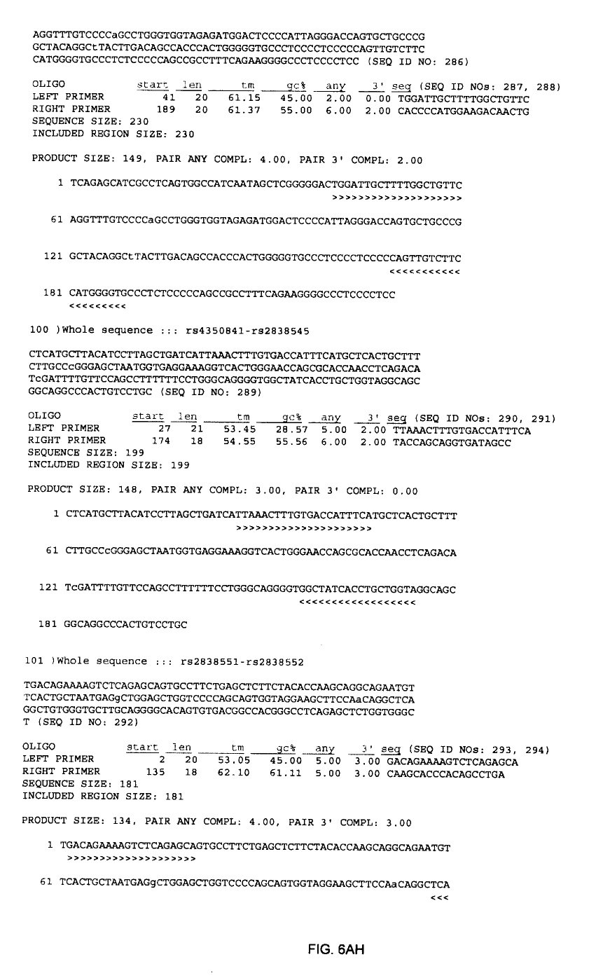

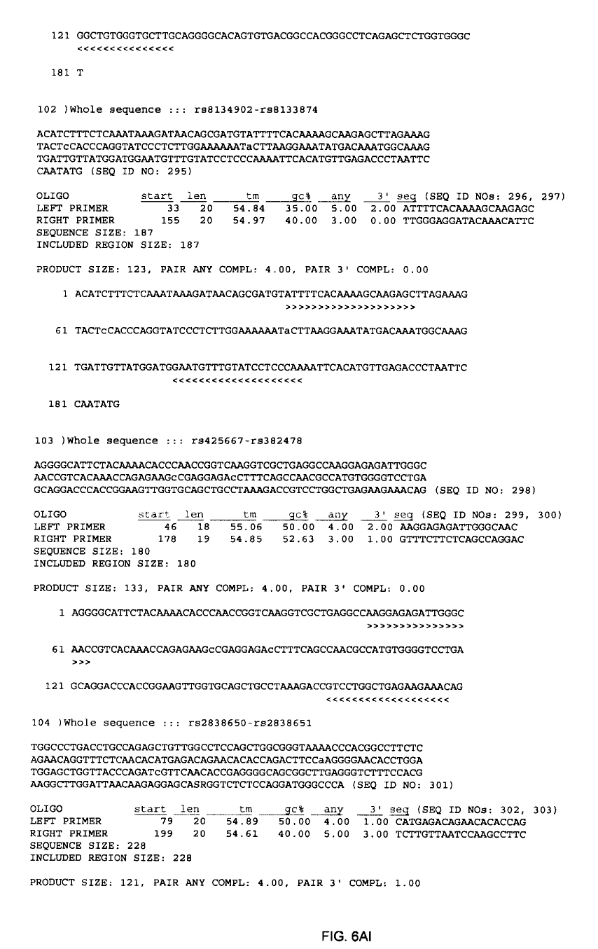

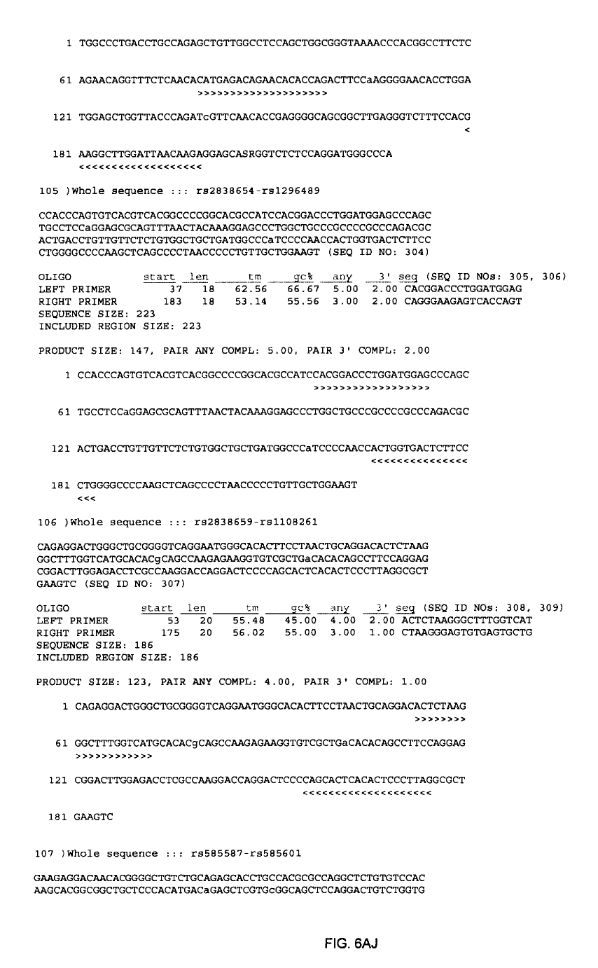

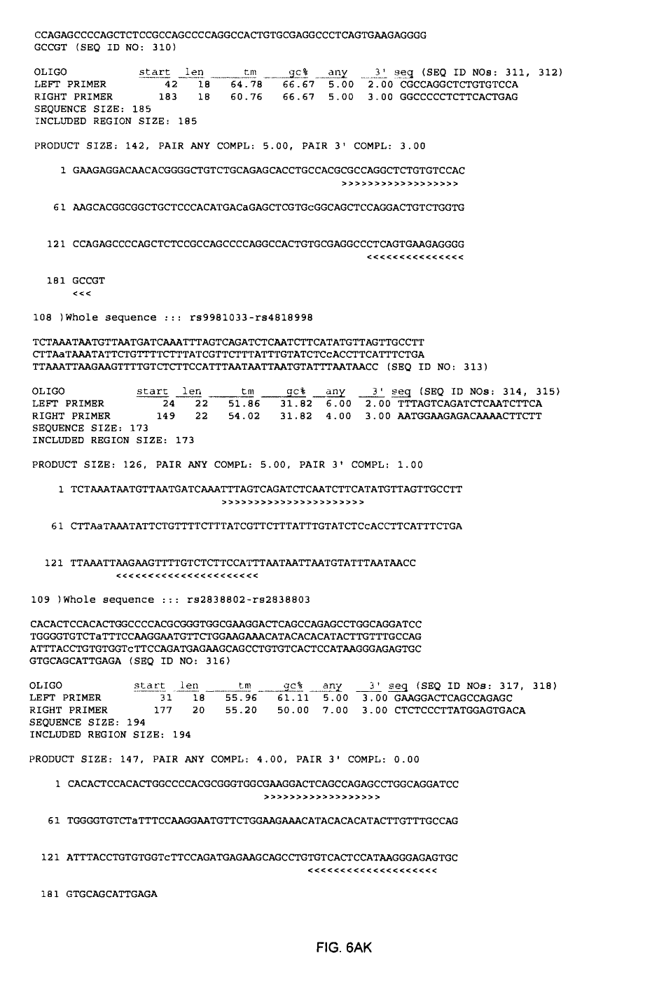

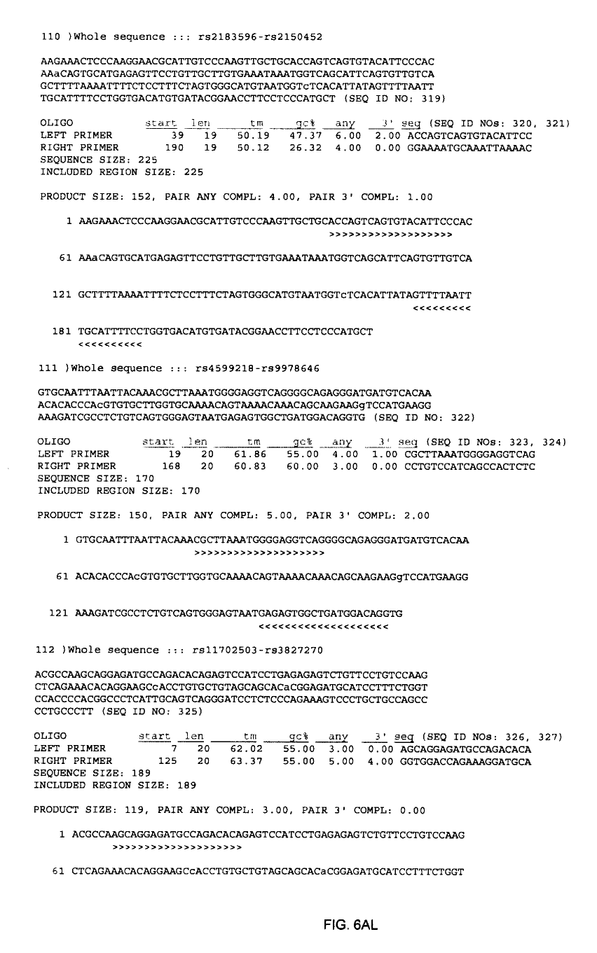

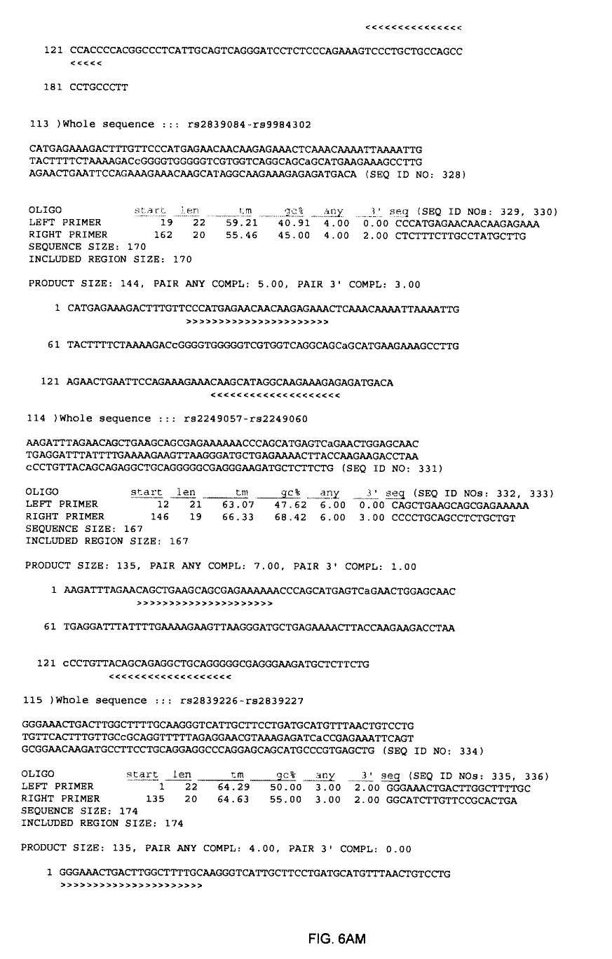

In order to increase the signal of alleles of interest for a specific chromosome or chromosomal region, the nucleic acids in a sample are in many embodiments amplified using methods known in the art. Such amplification methods include polymerase chain reaction (PCR), strand displacement amplification (SDA), multiple displacement amplification (MDA), rolling circle amplification (RCA), rolling circle amplification (RCR) and other amplification (including whole genome amplification) methodologies. In certain embodiments, the chromosomes are amplified using primers directed to desired regions. This amplification enriches the sample for the allelic sequences for those specific chromosomal regions, and then methods known in the art for detecting those sequences can be used to detect and quantify the different alleles present in the sample. In some embodiments all nucleic acids in the sample are amplified, and then alleles of interest are detected using methods known in the art and described herein. In some embodiments, alleles of interest are amplified using primers with sequences such as those listed in FIGS. 5 and 6 and then detected using methods well known in the art and described herein.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “nucleotide sequence” refers to a polymer of DNA or RNA which can be single-stranded or double-stranded, optionally containing synthetic, non-natural or altered nucleotide bases capable of incorporation into DNA or RNA polymers. The terms “nucleic acid,” “nucleic acid molecule,” or “polynucleotide” are used interchangeably herein.

In some embodiments, High-Fidelity (Hi-Fi) PCR is used to amplify alleles of interest in a sample. High-Fidelity PCR is an amplification method resulting in an error rate (in per basepair doubling) equal to or better than standard PCR. For example, Taq polymerase, which is not a high fidelity polymerase, has an error rate of ˜10⁻⁴ per basepair doubling. In contrast, Pyrococcus furiosus (Pfu) is a high-fidelity polymerase, with a published error rate for Pfu is 1.3×10⁻⁶ per basepair doubling (Cline et al, Nucleic Acids Res. 1996 Sep. 15; 24(18): 3546-3551). Examples of high-fidelity enzymes include Pfu and its derivations, or other enzymes with similar proofreading 3′->5′ exonucleases. Mixed blends and fusions with enzymes with proof-reading capabilities can increase the fidelity of a polymerase. Use of such a high fidelity polymerase ensures that the alleles of interest are amplified efficiently and with minimal to no introduction of errors.

Methods for improving PCR fidelity and efficiency include, among others: A) using a high-fidelity polymerase enzyme; and B) the addition of chemical reagents (e.g., betaine) that can lower temperatures required during the PCR process. Lowering temperatures required during the PCR products can increase efficiency and prevent damage to the amplification products, because prolonged heating of DNA and nucleotides during PCR can lead to damaged products, such as deaminated cytosines (uracils) and thus lead to misincorporation errors and miscopying errors during PCR (Andre, Kim, Khrapko, Thilly. Genome Res. 1997 7: 843-852. Zheng, Khrapko, Coller, Thilly, Copeland. Mutat Res. 2006 Jul. 25; 599(1-2):11-20).

In certain embodiments of the invention, amplification using HiFi-PCR, is performed with primers present in molar excess (e.g., 10¹² copies/μl of primer vs 10⁶ or less of the template) so that it is more likely that primers will anneal with template DNA than with each other (see, e.g., Li-Sucholeiki X C, Thilly W G. Nucleic Acids Res. 2000 May 1; 28(9):E44; Thompson J R, Marcelino L, Polz M. Nucleic Acids Res. 2002 May 1; 30(9): 2083-2088.). This can significantly reduce the creation of heteroduplexes.

### III. Tandem SNPs

In certain embodiments, comparisons of alleles as described above utilize alleles of tandem single nucleotide polymorphisms (referred to herein as “tandem SNPs”). A “single nucleotide polymorphism (SNP)” is a single basepair variation in a nucleic acid sequence. A “tandem SNP” is a variation in more than one nucleotide in a nucleic acid sequence, e.g. on a chromosome. In some embodiments, a tandem SNP is a pair of SNPs that are located in the nucleic acid sequence. In further embodiments, a tandem SNP comprises more than two SNPs. As will be appreciated, any number of SNPs can be contained in a tandem SNP, limited only by the total number of nucleotides present in the nucleic acid. In still further embodiments, a tandem SNP comprises from 2 to about 20 SNPs. In still further embodiments, a tandem SNP comprises from about 3 to about 19, about 4 to about 18, about 5 to about 17, about 6 to about 16, about 7 to about 15, about 8 to about 14, about 9 to about 13, and about 10 to about 12 SNPs. In yet further embodiments, a tandem SNP comprises between two SNPs and the maximum number of SNPS that can be assayed in a single reaction, which may depend on the detection platform (e.g., the read length of the sequencing technology). In still further embodiments, a tandem SNP comprises between two SNPs and the maximum number of SNPs contained within the segment of genetic material to be analyzed. For example, fetal DNA circulating in maternal serum is often fragmented and has a length of 200-400 bases—in such an exemplary embodiment, the number of SNPs contained in a tandem SNP would be limited by the maximum number of SNPs contained in that fragmented DNA.

As will be appreciated, the two or more single SNPs in a tandem SNP may be of any distance apart. For tandem SNPs with more than two single SNPs, the multiple SNPs may be of equal distance apart, or there may be varying distances between them. In some embodiments, the distance between SNPs of a tandem SNP is generally about 350 basepairs or fewer. In still further embodiments, the SNPs of tandem SNPs are about 5 to about 300, about 10 to about 250, about 15 to about 200, about 20 to about 150, about 25 to about 140, about 30 to about 130, about 35 to about 120, about 40 to about 110, about 45 to about 100, about 50 to about 90, about 55 to about 80, and about 60 to about 70 base pairs apart.

Increasing the number of SNPs that make up tandem SNPs of the invention increases the potential number of haplotypes, and thereby increases the likelihood that an assay detecting alleles will be informative. For example, if a tandem SNP contains 3 SNP sites, the potential number of haplotypes is 8 and if it contains 4 SNP sites, the potential number of haplotypes is 16. This increases the likelihood that an assay will be informative, because the mother is more likely to be heterozygous at such a tandem SNP, and the paternal contribution to the fetus (also referred to herein as the “paternally-inherited fetal allele”) is likely to be different from that present in the mother's genome. Thus, an assay that detects the alleles for this tandem SNP is likely to provide the at least three alleles that allows a comparison to be conducted for detection of a genetic abnormality, as is discussed in further detail herein.

Tandem SNPs provide a particularly powerful tool for comparing alleles in a sample, because tandem SNPs allow the detection and quantification of haplotypes. As will be discussed in further detail herein, the use of haplotypes in methods of the present invention provide an internal standard that eliminates the need for using reference markers across different chromosomes, thereby increasing specificity and/or minimizing or eliminating false positives.

As discussed herein, “haplotypes” are groups or sets of markers (including without limitation SNPs, deletions, small deletions, insertions, small insertions, methylation, and short tandem repeats) that are inherited together as a unit. Different alleles of a tandem SNP are thus haplotypes, and haplotypes can be assigned to a chromosome (i.e., the maternally inherited or paternally inherited chromosome). Comparing alleles of a tandem SNP comprises far more information than is possible from comparing individual SNPs, because comparing alleles of a tandem SNP provides a comparison of haplotypes.

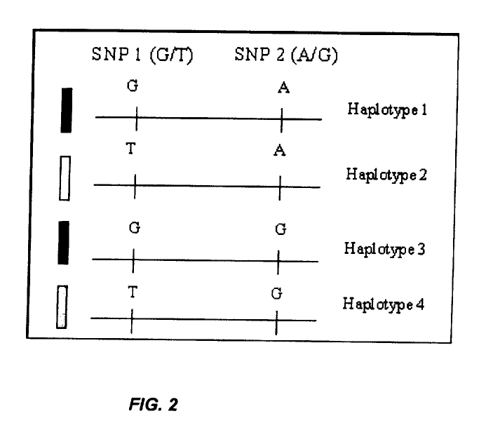

An exemplary embodiment of a tandem SNP is schematically illustrated in FIG. 2. In this embodiment, the tandem SNP comprises a pair of SNPs. At SNP 1, two alleles are possible: G or T. At SNP 2, two alleles are possible: A or G. There are therefore four alleles possible for this tandem SNP, and these alleles are defined by the four possible haplotypes: G-A, T-A, G-G, or T-G. As will be appreciated, an individual may be heterozygous or homozygous for a tandem SNP, as is the case for a single SNP, but being heterozygous for a tandem SNP means that multiple different haplotypes are possible alleles for that tandem SNP. As will also be appreciated, for tandem SNPs containing three or more SNPs, the resultant number of possible haplotypes will also increase.

As discussed above, genetic abnormalities can be detected in a fetus by comparing the number of molecules of alleles between the fetal and maternal nucleic acids in a sample. By using tandem SNPs, the comparison can be used to both compare and quantify the differences between maternal and fetal DNA.

In an exemplary embodiment, detecting the alleles of tandem SNPs in a sample containing both maternal and fetal DNA can be used to calculate a haplotype ratio. As discussed above, these allele comparison assays are conducted for chromosomal regions of interest at which the maternal genome is heterozygous. In the case of tandem SNPs, the maternal genome being heterozygous means that it has two different haplotypes for that particular tandem SNP. Detecting the number of molecules of alleles of the tandem SNP present in a sample is thus a detection of the number of molecules of the different haplotypes are present in the sample.

If three alleles of a tandem SNP are detected in a sample containing both maternal and fetal DNA, the haplotype ratio (HR) can be calculated using the following formula:

HR=|P1−P2|/P3 (Formula I)

P1 and P2 represent the number of molecules of each of the maternal haplotypes, and P3 represents the number of molecules of a third haplotype that is not present in the maternal genome. P3 thus represents the number of molecules of the haplotype inherited by the fetus from the father. As discussed herein for comparing alleles, the calculation of the haplotype ratio provides a way to determine if the fetus has a chromosomal abnormality, including without limitation trisomy, monosomy, partial duplication, partial deletion, microduplication, microdeletion, and the like. As will be appreciated, the description of the number of molecules of alleles as relating to the terms “peak”, and “x” are related to P1, P2 and P3 of Formula 1. For example, P1 and P2 represent the maternal alleles—i.e., “peak+x” (or “peak+2x” in cases of trisomy) and “peak”, whereas P3 of Formula 1 is equivalent to the paternally-inherited fetal allele, i.e., “x”.

In exemplary embodiments, the haplotype ratio calculation will produce the discrete results of 1, 2, 0 or 0.5. These numbers are interpreted as follows:

TABLE 1
interpretation of haplotype ratio calculations for the detection
of trisomy and/or partial duplications

| Haplotype ratio | Interpretation |
|---|---|
| \|P1 − P2\|/P3 = 1 | Fetus has the normal number of chromosomes |
| \|P1 − P2\|/P3 = 2 | Fetus has trisomy, likely as result of a duplication |
| | during maternal meiosis II or paternal meiosis I |
| \|P1 − P2\|/P3 = 0 | Fetus has trisomy, likely as a result of a |
| | duplication during maternal meiosis I |
| \|P1 − P2\|/P3 = 0.5 | Fetus has trisomy, likely as a result of a |
| | duplication during paternal meiosis II |

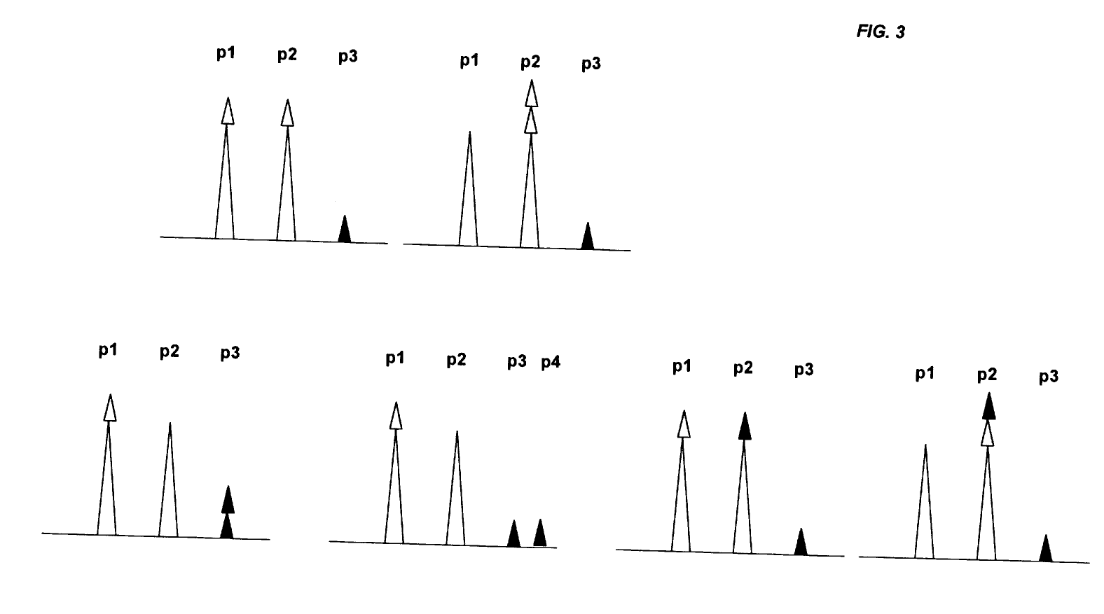

In a further embodiment, paternal non-disjunction, in which the fetus inherits two alleles from the father, may result in the haplotype ratios described in Table 1. In addition, if the two alleles from the father are both not found in the maternal genome, it would be possible to detect four alleles in a sample containing both maternal and fetal DNA—two alleles would again be from the maternal genome, and two would be paternally-inherited fetal alleles (see FIG. 3, lower panel for exemplary combinations of alleles).

In further embodiments, the haplotype ratio can be calculated according to the following formula:

HR=|P1−P2|/|P3+P4| (Formula II)

In this embodiment, P1 and P2 represent the number of molecules of each of the maternal haplotypes and P3 and P4 represent the number of molecules of a third and fourth haplotype that is not present in the maternal genome. As will be appreciated, Formula II is applicable to embodiments of paternal non-disjunction trisomy, in which two paternally-inherited fetal alleles are present in the sample. Formula II is also applicable to situations in which only one paternally-inherited fetal allele is present in the sample—in such situations, either P3 or P4=0. Thus, Formula II can be applied to calculate the haplotype ratio for any sample from a pregnant female carrying a single fetus.

For de novo deletions in the fetus (including partial deletions, microsomal deletions, large deletions, etc,), paternally inherited deletions in the fetus would result in an entire panel of tandem SNPs without an informative third allele being present in the maternal sample. In such a situation, haplotype ratios cannot be calculated. Thus, the absence of an informative third allele or third haplotype can indicate a de novo deletion in the paternally inherited chromosome and would be informative in and of itself.

Thus, calculation of the haplotype ratio provides discrete detection and quantification of fetal chromosomal/genetic abnormality/allelic dosage within a single measurement from a sample comprising both maternal and fetal DNA. Unlike traditional methods of using single SNPs to determine whether a fetus has a chromosomal abnormality, the present invention does not require measurements of alleles across different chromosomes in order to determine the amount of fetal DNA present in the sample in relation to the maternal DNA. As discussed above, the present invention has an internal standard for each measurement. Since the maternal genome is heterozygous for the particular tandem SNP being analyzed, detection of three alleles in a sample containing both maternal and fetal DNA means that one of the detected alleles is the paternally-inherited fetal allele. Since the molecules of the paternally-inherited fetal allele can only be from the fetal DNA, detection of the number of molecules of that paternally-inherited fetal allele provides a measurement of how much fetal DNA is present in the sample. This third allele serves as an internal standard of the amount of fetal DNA, and there is no need to compare the measurement of the number of molecules of fetal alleles against the number of molecules of fetal alleles present for another chromosome that is not expected to have an abnormality. Thus, in the present invention, a single measurement of the number of molecules of an allele of a particular chromosome (i.e., a chromosome expected to be subject to an abnormality) provides all the information needed to determine whether the fetus has a chromosomal abnormality.

A further advantage of the methods of the present invention, particularly the internal standard provided by detection of the third allele, is that the ratios of the alleles and the calculation of the haplotype ratio will not vary based on the concentration in the sample of fetal genetic material. Traditional methods for detecting chromosomal abnormalities must normalize calculations of ratios with respect to the amount of fetal genetic material present in the sample being analyzed. In contrast, the methods of the present invention do not require such normalization, because comparing the number of molecules of the paternally-inherited fetal allele to the number of molecules of the maternal alleles in accordance with the present invention allows a calculation of allelic ratios that is independent of the amount of the total amount of fetal genetic material present in the sample.

As will be appreciated, the above methods for calculating haplotype ratios can be conducted using a single tandem SNP or using multiple tandem SNPs (also referred to herein as “panels” of tandem SNPs). When multiple tandem SNPs are used in any methods in accordance with the present invention, they can be utilized one at a time, in specific groupings, or all possible tandem SNPs can be analyzed at the same time in a multiplex assay. In some embodiments of the invention, panels of tandem SNPs comprise between 2 and 200 tandem SNPs. As will be appreciated, each of these tandem SNPs define a haplotype and therefore may in turn comprise two or more SNPs. In further embodiments, panels of tandem SNPs comprising between about 2-150, 10-140, 15-130, 20-120, 25-110, 30-100, 35-90, 40-80, 45-70, and 50-60 tandem SNPs are used in accordance with the present invention.

Multiple tandem SNPs may be assayed individually for a sample or multiplexed into a single assay. In some embodiments, the number of SNPs applied in a single assay may depend on the amount of nucleic acids that are present in and/or can be obtained from a sample. In further embodiments, all or a selected portion of the nucleic acids in a sample are amplified, which will allow multiple tandem SNPs directed to one or more chromosomes to be applied in a single multiplexed assay. As will be appreciated, using multiple tandem SNPs, either sequentially or simultaneously, can be used in some embodiments to assay for abnormalities in multiple chromosomes. The number of chromosomes that can be assayed using methods of the invention is limited only by the number of chromosomes present in the organism from which the sample is obtained.

In many embodiments, a panel of tandem SNPs is assayed in a sample, but only a subset of the panel (anywhere from between 1 and up to the total number of tandem SNPs in the panel) is informative, meaning that not every tandem SNP in a panel will produce a “positive” result. A positive result in assays utilizing tandem SNPs is generally the detection of at least three alleles for a particular tandem SNP. For any particular sample, even a single positive result from a tandem SNP can allow detection of a genetic abnormality. Results from more than one tandem SNP can be used as further internal confirmation of an assay.

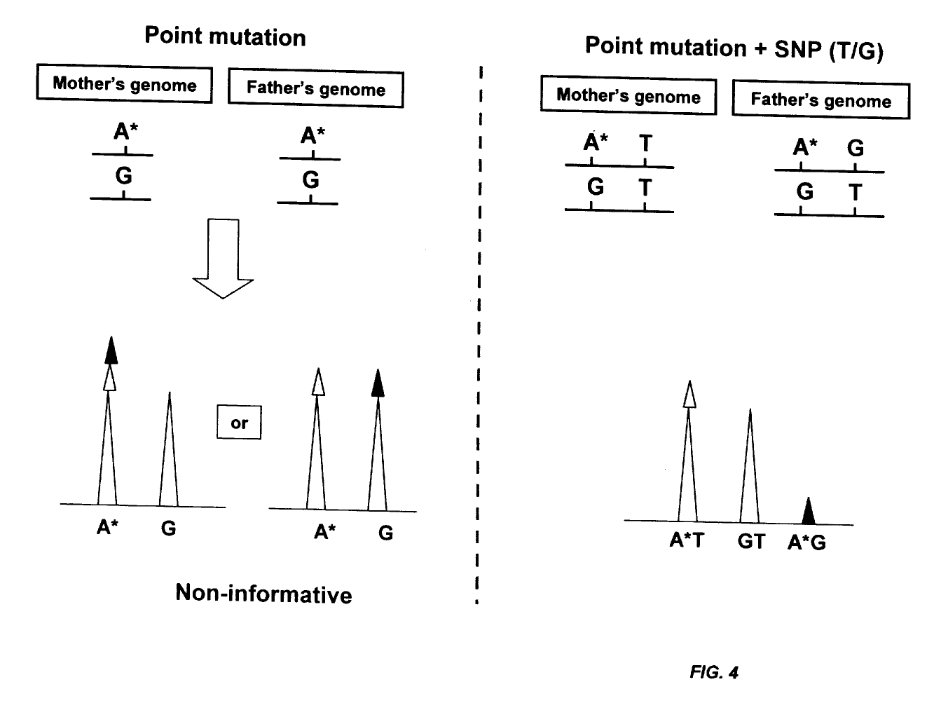

The analysis of the paternally inherited fetal allele, particularly analysis with respect to tandem SNPs, is of use in detecting non-chromosomal mutations. In the case of a point mutation that is present in the mother or father's genome (A* in FIG. 4), this can be combined with a nearby SNP site to create at least three potential haplotypes. The inheritance pattern of the point mutation(s) to the fetus can therefore be traced in accordance with the methods described herein.

As is schematically illustrated in FIG. 4, the use of tandem SNPs allows detection of a recessive mutation that would not be detectable in methods utilizing single SNPs. As shown in the left panel, detecting alleles for a single SNP in a sample comprising both maternal and fetal DNA does not allow one to determine whether the fetus has inherited the point mutation. However, using the haplotype analysis possible by detecting alleles of tandem SNPs (FIG. 4, right panel), it is possible to detect the third allele in the sample, which is the paternally-inherited fetal allele. If the paternally-inherited fetal haplotype comprises the recessive point mutation, detection of that third allele is informative. Although in this exemplary embodiment the tandem SNP comprises two single SNPs, as is discussed herein, tandem SNPs may comprise multiple single SNPs.

In the extremely rare situation that the point mutation occurs at a base pair which is known to have a SNP, the single site would be tri-allelic and therefore it may not be necessary to combine the point mutation site with a nearby SNP. Then a simple comparison of detected alleles as described in further detail above would allow detection of the mutant allele.

The ability to trace the inheritance pattern of point mutations has clinical importance in the case of recessive disorders. If both the mother and father are heterozygous at a point mutation site carrying a normal and mutated base, the use of haplotypes (point mutation+nearby SNP) allows one to determine whether the fetus is at risk for being homozygous for the recessive mutation. This is thus one embodiment of the tandem SNP analysis described herein. The combination of any mutation (e.g. point mutation, insertion, deletion, methylation) along with a nearby SNP is useful to determining the fetal risk of homozygosity for a recessive disorder.

In some embodiments, short tandem repeats (STRs) can be used as a tandem SNP or as part of a tandem SNP for comparing alleles. STRs are highly polymorphic regions in the genome, and many alleles can be present at a single STR site—for example, in many situations it is possible to have ten or more alleles at a given site. STRs generally arise in highly repetitive sequences which are error prone during amplification, resulting in their highly polymorphic state.

In some embodiments, tandem SNPs do not comprise STRs.

In some embodiments, the information gathered about the alleles of interest in a sample comprising both maternal and fetal DNA is compared to similar information gathered about the alleles of interest in a sample comprising maternal DNA but no fetal DNA. The sample containing only the maternal DNA can be used as a reference for the sample containing both maternal and fetal DNA. Although such a reference sample is not generally necessary, it can provide additional substantiation of the results from the assays discussed herein. In some embodiments, the sample comprising maternal DNA but no fetal DNA is obtained using methods known in the art. Such samples can be obtained from a number of sources, including without limitation: maternal buccal swabs, maternal cells, including maternal white blood cells, and the like.

III(A). Identifying Tandem SNPs

As will be appreciated, different samples will require the use of different tandem SNPs for detection of genetic abnormalities. This is because one requirement of the methods described herein, particularly methods involving tandem SNPs, is that the maternal genome must be heterozygous for the allele of interest in order to allow the detection of a third, paternally-inherited allele. In addition, tandem SNPs for which the paternal genome comprises the same haplotypes as the maternal genome will not be informative, because it is the detection of three alleles for a particular tandem SNP that provides the internal standard of the paternally-inherited fetal allele.

The present invention provides methods and compositions for identifying tandem SNPs. In some embodiments, a chromosomal region of interest is studied to identify potential markers (i.e., alleles) for use as tandem SNPs. Studying a chromosomal region of interest for identifying tandem SNPs in some embodiments involves analyzing a database comprising information on the occurrence of SNPs in one or more populations (such as the database from the International HapMap Project). In further embodiments, studying a chromosomal region of interest to identify tandem SNPs comprises collecting samples of nucleic acids for a number of individuals and amplifying the region of interest. The amplification product can then be sequenced or otherwise analyzed to identify potential markers for use as tandem SNPs.

In some embodiments, a tandem SNP is chosen by identifying two or more neighboring SNPs. Such SNPs may be separated by any range of distances, and as will be appreciated, the distances between neighboring SNPs is limited only by the size of the chromosomal region of interest. In some embodiments, the two or more SNPs in a tandem SNP may be separated by a distance of about 5 to about 400 base pairs. In further embodiments, the two or more SNPs in a tandem SNP may be separated by a distance of about 10 to about 350, about 20 to about 300, about 30 to about 250, about 40 to about 200, about 50 to about 150, about 50 to about 140, about 60 to about 130, about 70 to about 120, about 80 to about 110, and about 80 to about 100 base pairs. In embodiments in which more than two SNPs are in a tandem SNP, each individual SNP may be of equal distance from the other SNPs, or the SNPs may be at varying distances from each other. Although the description herein of tandem SNPs is primarily in terms of two or more SNPs, a tandem SNP may also include any combination of SNPs, deletions, insertions, and sequence variants of two nucleotides or greater, and descriptions of tandem SNPs herein applies to all such combinations. As will be appreciated, “neighboring” SNPs as described herein may have one or more SNPs (or deletions, insertions, sequence variants) that occur in the region between the SNPs that are part of a particular tandem SNP. Thus, in an exemplary embodiment, if SNPs A, B and C occur in that order along a chromosomal region in a population, a tandem SNP may be chosen such that A and C are the “neighboring” SNPs, and B is a SNP located in the intervening region between A and C.

In further embodiments, tandem SNPs of the invention are selected such that the combination of SNPs (and/or deletions, insertions and sequence variants) results in more than two haplotypes being present in a population. In still further embodiments, tandem SNPs are chosen that result in more than two haplotypes present in populations across different ethnic groups—as will be appreciated, such information can be generated from publicly available databases (such as the HapMap database) or from direct investigation of samples collected from multiple individuals.

In still further embodiments, tandem SNPs of the invention are chosen such that their component SNPs, deletions, insertions and sequence variants do not lie within a common, non-disease related CNV (copy number variation) region, such as those present in the Database of Genomic Variants from The Centre for Applied Genomics at http://projects.tcag.ca/variation/.)

In further embodiments, methods for identifying tandem SNPs utilize maternal buccal samples (or any other samples that contain maternal DNA but no fetal DNA) in order to identify regions at which the maternal genome is heterozygous.

In an exemplary embodiment, tandem SNPs are identified from population data files (such as the HapMap database). In this exemplary embodiment, highly heterozygous (i.e., greater than 10% or more or “common”) SNPs are identified. In some embodiments, only those SNPs that are highly heterozygous in all populations contained in the database are chosen as likely candidates. In other embodiments, likely candidates are chosen from SNPs that are highly heterozygous in a subset of the populations contained in the database. Tandem SNPs can then be provisionally selected by choosing highly heterozygous SNPs that are at a desired distance from each other, as is discussed above. As is also discussed herein, likely tandem SNPs may comprise a wide range of individual SNPs.

In still further embodiment, any likely tandem SNPs that occur in the list of SNPs from common CNV regions are removed from further consideration. In still further embodiments, tandem SNPs that lie in regions with long stretches of homopolymeric sequences (i.e. 6 or greater) are also removed from further consideration.

In yet further embodiments, primers are designed to amplify regions containing likely tandem SNPs. In some embodiments, these primers are optimized for efficiency and specificity using methods known in the art. In further embodiments, primers are tested to determine if they preferentially amplify one or more alleles of the likely tandem SNPs over others.

As will be appreciated, tandem SNPs of use in accordance with the present invention can be chosen using any combination of the above methods and characteristics. In some exemplary embodiments, tandem SNPs are chosen based solely on distance between each component. A “component” of a tandem SNP as used herein refers to individual SNPs, deletions, insertions, or sequence variations that make up a tandem SNP.

In some embodiments, a chromosomal region of interest can be directly sequenced from one or more samples to identify potential tandem SNPs. In further embodiments, genetic material from a number of individuals, (e.g., 8 individuals, 10 individuals, 96 individuals, even 20,000 individuals) can be collected and amplified for a given target sequence of interest. The alleles present in this amplified sample can then be detected and quantified to identify sequences that can serve as tandem SNPs. In some embodiments, the genomic DNA from these individuals are pooled together and a target sequence of interest is amplified in a single PCR reaction. If three or more alleles occur in this pooled sample, and at least three of these alleles occur at high percentages (>10%) in the pooled sample, then the target sequence is a likely candidate for use as a tandem SNP in accordance with the present invention.

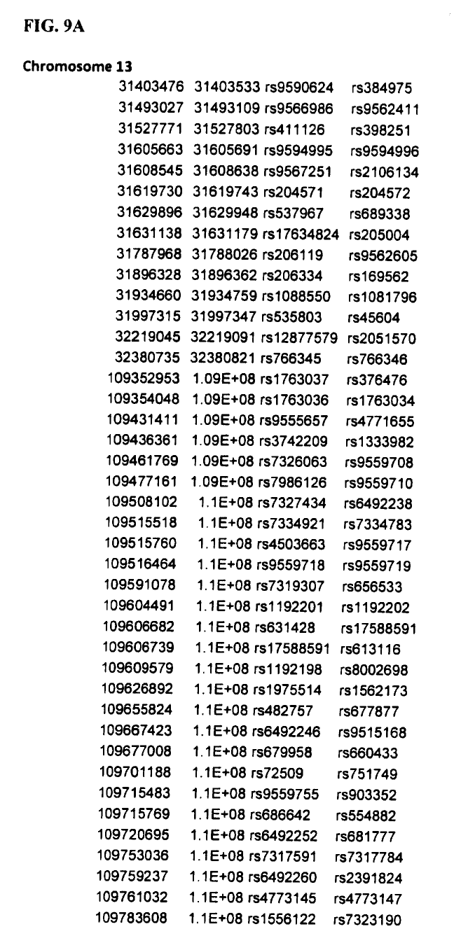

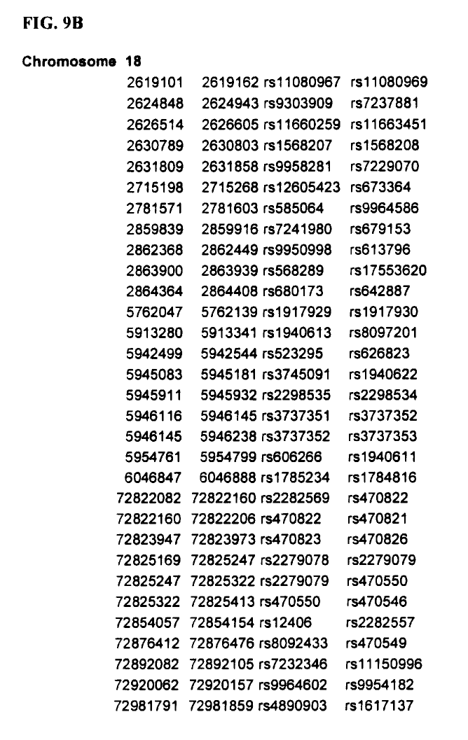

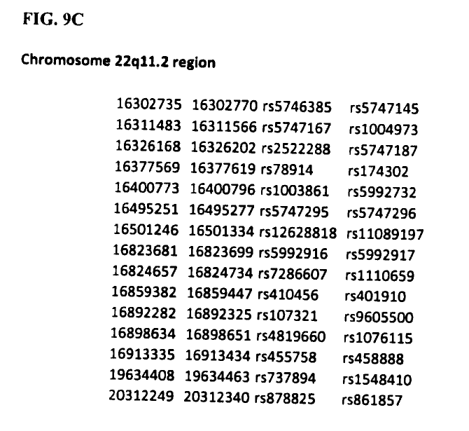

As will be appreciated, tandem SNPs can be identified for any chromosome. Exemplary tandem SNPs identified in accordance with the present invention for chromosome 21 are provided in FIG. 5. FIG. 6 shows tandem SNPs for chromosome 21 as well as primers that can be used for amplification and detection of these tandem SNPs. Exemplary tandem SNPs identified in accordance with the present invention for chromosomes 13, 18 and 22 are provided in FIG. 9. Tandem SNPs identified in accordance with the present invention for any other chromosome are also encompassed by the present invention, as are tandem SNPs other than those in FIGS. 5, 6 and 9. As will be appreciated, sequences that have at least 65%, 70%, 75%, 80%, 85%, 90%, 91%, 92%, 93%, 94%, 95%, 96%, 97%, 98%, and 99% sequence identity to the sequences provided in FIGS. 5-6 and 9 are also encompassed by the present invention.

### IV. Uses Beyond Detecting Genetic Abnormalities

Although the above description has been provided in terms primarily of genetic abnormalities, the same detection and quantification of alleles, including alleles of tandem SNPs, can be used in other applications.

In some embodiments, the present methods and compositions of the present invention can be used to evaluate tissue rejection in a patient who has received a tissue transplant. In such embodiments, if the host is heterozygous, detection of a third (and possibly fourth) allele frequency may come from the graft at the time of transplant. The levels of allelic dosage at the time of transplant can then be compared to levels at later time points to determine if the graft is being rejected. Such comparisons can be conducted by calculating allelic and/or haplotype ratios as discussed herein. An advantage of using tandem SNPs in this embodiment, particularly with tandem SNPs comprising more than two SNPs or other components, is that the likelihood of having an informative assay (i.e., one in which at least three alleles can be detected) is increased, and it would be possible to quantitatively measure both the maternally inherited and the paternally inherited alleles from both the recipient and the graft by calculating a Haplotype Ratio. Such information can not be obtained from studies in which only individual SNPs are used to compare between the genetic material of the recipient and the graft.

In still further embodiments, the methods and compositions can be used in transfusions, to determine if a donor is a match for the recipient. As with the analysis for detecting tissue rejection, the allelic dosage can be calculated to determine if the transfusion is acceptable to the recipient.

In further embodiments, the methods and compositions of the present invention can be used in forensic analysis by identifying samples through a comparison of alleles for a particular chromosomal region. Methods of the present invention for detecting and comparing alleles, including alleles of tandem SNPs, will provide information as to whether a particular forensic sample comprises samples from one or more individuals. Such forensic analysis may be used in cases of sexual assault, for determining paternity, as well as other forensic applications that would benefit from the ability to compare allelic dosage in a sample.

### EXAMPLES

### Example 1

### Tandem SNPs for Chromosome 21

Allelic markers on chromosome 21 were selected by examining tandem SNPs. These tandem SNPs covered both q and p arms of the chromosome. Using heterozygosity data available through dbSNP, DCC Genotype Database and through the HapMap Project, SNPs that appeared to be promising for high heterozygosity (≧25%) were selected. Because all four possibilities may not exist in nature due to haplotype blocks in regions of low recombination, those that suggested less than three haplotypes were screened out.

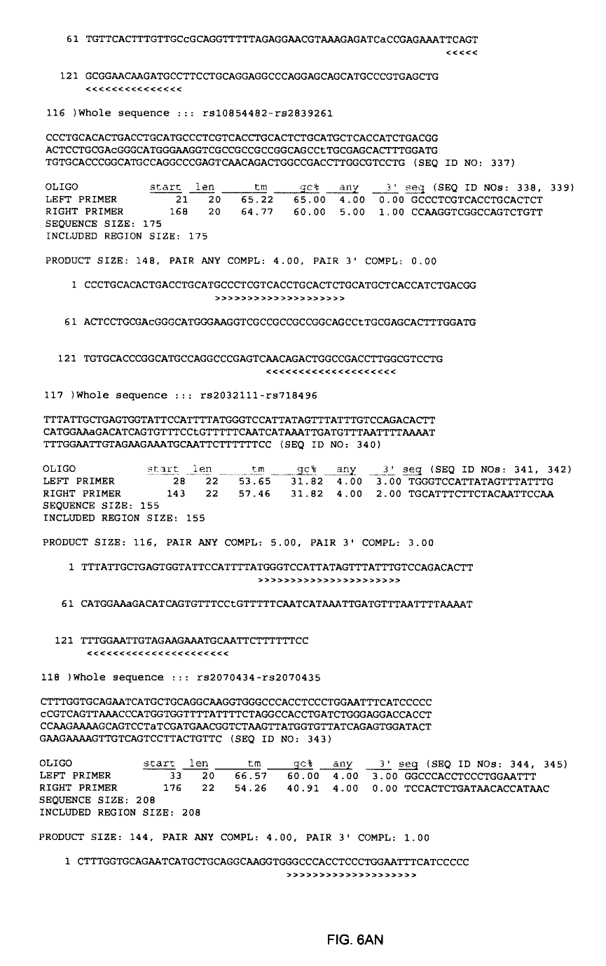

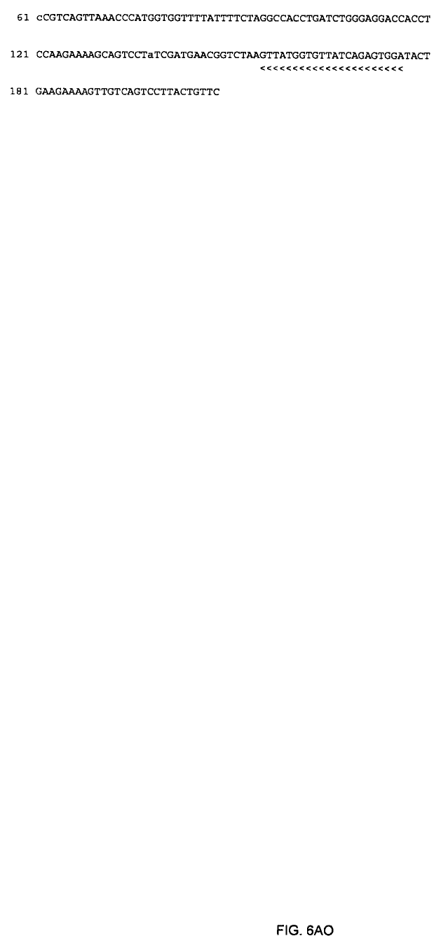

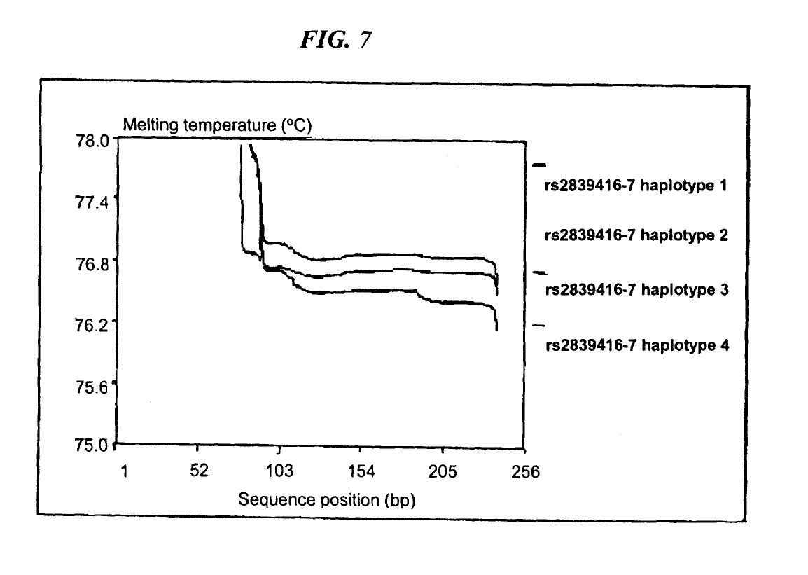

Target sequences covering tandem SNPs were designed using Vector NTI and WinMelt software. As an example, the melting map of a CDCE or CTCE target covering two tandem SNPs (dbSNP rs2839416 and rs2839417) on chromosome 21 was calculated using WinMelt according to the algorithm of Lerman and Silverstein (Lerman et al., Methods Enzymol, 1987. 155: p. 482-501) and is depicted in FIG. 7.

FIG. 7 depicts a DNA melting map of a CDCE or CTCE target sequence covering tandem SNPs. All four haplotypes can be theoretically separated according to DNA melting temperature. The curves for the four different haplotypes (haplotype 1 (G,A); haplotype 2 (T,A); haplotype 3 (G,G); and haplotype 4 (T,G)) are identified on the figure.

HiFi PCR optimization for each target sequence was performed using Pfu polymerase. One of primers flanking the target sequence was ˜20 bases in length and labeled 5′ with a fluorescein molecule. The other primer was about 74 bases including a ˜20-base target specific sequence and the 54-base clamp sequence. A standard HiFi PCR condition was applied to all target sequences, varying only annealing temperatures. These PCR amplicons were subjected to CDCE or CTCE electrophoretic separation. The resulting electropherograms were analyzed for yield and purity of the PCR products. The purity was evaluated by comparing the peak area of the desired products to that of the byproducts and nonspecific amplification. Target sequences that could be amplified with a high PCR efficiency (≧45% per cycle) and low levels of byproducts and nonspecific amplification (≦0.1% of the desired products) were subjected to CDCE or CTCE optimization. For those target sequences that did not have acceptable PCR products in the first stage, increasing amounts of Mg⁺² concentrations (up to about 7 mM) in combination with different annealing temperatures were tested. For the remaining target sequences that still did not work, primer positions were changed and the entire optimization process is repeated.

For CDCE or CTCE optimization, the relevant haplotypes were created for the targets using pools of 96 individuals. The optimal separation condition for each haplotype should provide the greatest resolution among the observed peaks. Initial optimization is done around the theoretical melting temperature (Tₘ) in a 2° C. temperature range in increments of 0.2° C. which covers (Tₘ−1° C.±a predetermined offset) to (Tₘ+1° C.±a predetermined offset).

Electropherogram and peak measurements were transferred to a spreadsheet for analysis. To ensure the quality of the data, minimum and maximum peak heights were used. Individual markers were failed if electrophoretic spikes occur. Peak areas were used to calculate allele ratios. A check for allelic preferential amplification was performed on all 96 tandem SNPs.

In the fall of 2005, the International HapMap Project publicly released genotypes and frequencies from 270 people of four ethnic populations. Chromosome 21 haplotype data from approximately 40,000 SNPs genotyped across four populations, including U.S. residents with northern and western European ancestry, residents of Ibadan, Nigeria, of Tokyo, Japan, and of Beijing, China, were downloaded (2005-10-24: HapMap Public Release #19) and converted to the +orientation. Tandem SNP candidates fell within 100 basepairs from each other and at least three haplotypes existed in all four ethnic populations. CDCE or CTCE target sequences and primers were designed for the tandem SNPs identified through the HapMap Project. The neighboring sequences for each of the tandem SNPs were imported into a software program, e.g., Sequencher (Gene Codes, Ann Arbor, Mich.) and/or Vector NTI (Invitrogen, Carlsbad, Calif.) for sequence alignment and primer design, and into Winmelt (Medprobe, Oslo, Norway) or Poland software (available at biophys.uniduesseldorf.de/local/POLAND/poland.html) where the algorithm for computing DNA melting temperatures given the Gotoh-Tagashira values for the enthalpy of melting DNA sequences were used to calculate melting temperatures of target sequences. CDCE or CTCE candidates generally have a high melting region adjacent to a low melting region, lie in a low melting region, melting temperatures of the low melting region fall below 80° C., and no “valleys” occur between the high melting region and the low melting region.

All of the 40,000 genotypes on chromosome 21 were analyzed for tandem SNP/CDCE/CTCE marker suitability. 118 tandem SNPs/CDCE/CTCE targets meeting requirements have been identified (see FIG. 5 for the first 42 identified and FIG. 6 for all 118).

Primer sequences for these 118 tandem SNP/CDCE/CTCE targets were designed. These were optimized as described herein using HiFi PCR and CDCE or CTCE. These optimizations included the creation of relevant haplotypes for all targets, a check for allelic preferential amplification during HiFi PCR, and obtaining the greatest resolution among peaks during CDCE or CTCE. Haplotypes may be separated as homoduplex peaks. However, if certain targets cannot be separated out as homoduplexes, maternal DNA can be separated from fetal DNA as heteroduplexes.

### Example 2

### Determining Heterozygosity of Tandem SNPs

Genomic DNA samples from 300 anonymous subjects were obtained from healthy young adults who were less than 35 years old. The samples were anonymous as the only data obtained were the geographic location of the Red Cross blood donor center, donor gender, and whether or not the donor was 35 and under. These samples were reviewed to ensure that at least three haplotypes were present for a given target sequence of interest. These results were compared to haplotypes identified through analysis of the database from the HapMap project as described in Example 1, and it was found that the same or similar haplotypes were identified using both methods.

### Example 3

### Detecting Fetal DNA in Maternal Serum

A cohort of subjects confirmed to have trisomy 21 by traditional karyotype analysis was examined. Tandem SNPs were used to demonstrate detection of trisomy in subjects. DNA from 20 subjects who were characterized by traditional karyotype analysis to have trisomy 21 were analyzed with the tandem SNP panel.

Biological samples, including a buccal (cheek) swab and a blood sample were collected from a cohort of pregnant women. Maternal buccal swab samples were compared to maternal serum to demonstrate that a third (paternal) peak was observed in several of the tandem SNP assays. Approximately 20 maternal buccal swab to maternal serum comparisons were made. To control for experimental artifacts, genomic DNA samples from maternal buccal swabs were utilized for each target sequence. The buccal samples were subjected to the process in parallel with the maternal blood sample. Any artifacts generated by the CDCE/CTCE/HiFi-PCR procedure (including nonspecific PCR amplification and polymerase-induced mutations) were revealed as background peaks in the buccal swab samples.

### Example 4

### Detecting Fetal Chromosomal Abnormalities

A blinded study is performed where the goal is to detect 20 known trisomy 21 fetuses by assaying maternal serum from 40 patients (previously determined by amniocentesis or CVS) (see FIG. 3).

FIG. 1A depicts a schematic illustration of the output of detecting alleles in a sample from maternal buccal swab. Markers exhibiting two alleles were pursued. A baby with trisomy is expected to show either three alleles, evident by three peaks in a 1:1:1 ratio or two alleles in a 2:1 ratio. FIG. 1B depicts a sample from maternal serum. Markers exhibiting three alleles are informative. Maternal serum from a woman carrying a baby with trisomy is expected to exhibit three alleles, evident by two equal peaks with a third smaller peak if the trisomy occurred during meiosis I (75% of T21 cases) or three alleles with different areas if the trisomy occurred during meiosis II (20% of T21 cases) where areas are: peak, x, and peak+2x. FIG. 1C is a schematic illustration of the analysis of a sample from maternal serum. Markers exhibiting three alleles are informative. Maternal serum from a woman with a normal baby with three alleles has three different areas where areas are: peak, x, and peak+x.

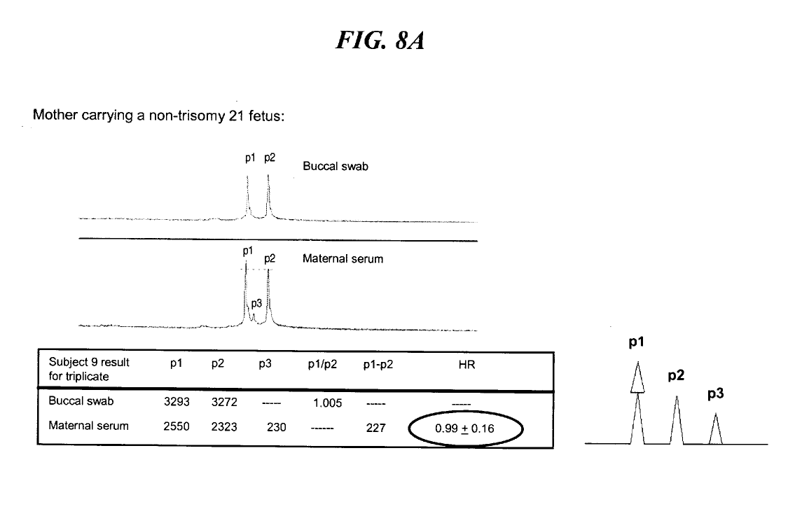

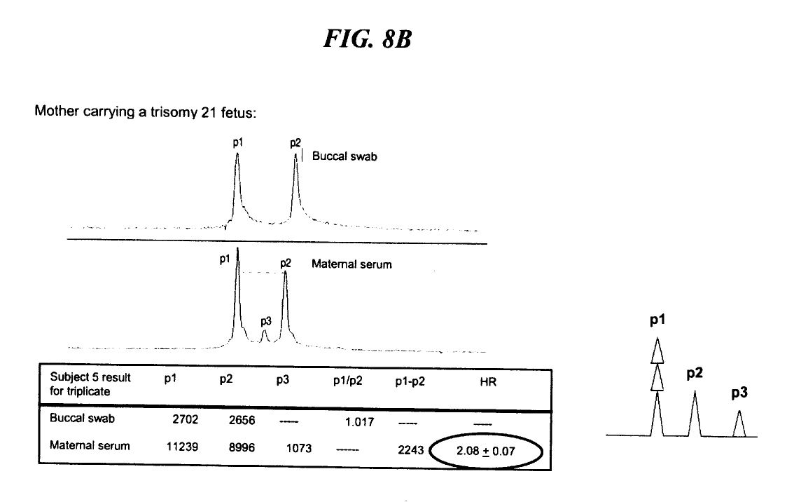

FIG. 8 shows data from a CDCE experiment for two different samples. In the top sample, the mother is carrying a non-trisomy 21 fetus. As expected, the haplotype ratio calculated from comparing the peaks in the output (which each represent a different allele, and the area under each of which provides the number of molecules for each allele) is within a margin of error to 1, the haplotype ratio expected for a normal fetus (see FIG. 8A). FIG. 8B shows the results for a mother carrying a fetus with trisomy 21. The haplotype ratio calculated from these data was within a margin of error to 2, which is one of the haplotype ratios expected for a fetus with a chromosomal abnormality.

For the case of the minimum heterozygosity, where both SNP1 and SNP2 are heterozygous at their respective loci at a rate of 25%, if 96 tandem SNPs are assayed, an average of 43 markers (44.5%) are expected to be heterozygous (two haplotypes) in the mother. The mother's expected heterozygosity is calculated using the following formula:

H=1−Σpᵢ² (Formula III)

wherein I=1 to k alleles and pi=estimated allele frequency.

The allele frequencies at each SNP loci are expected to be 85% and 15% for the majority and minority alleles, respectively, assuming Hardy-Weinberg equilibrium. The desired third haplotype is expected to be present at an average of 6.4 markers (15%) of per maternal-fetal sample tested. Because most loci have a heterozygosity value greater than 25%, for every maternal-fetal sample tested using the panel of 96 tandem SNP assays, greater than about 6.4 markers are most informative. Thus, while a panel of 96 tandem SNPs may be used, 6 or 7 of those tandem SNPs may be informative for any one specific maternal-fetal sample tested, and a ‘positive’ result from any one of those tandem SNPs is informative.

Finally, in order to diagnose a trisomy, a “positive” tandem SNPs should be identified on both the p and the q arm of chromosome 21. Because of the comparative nature of the basic approach, the tandem SNP assay is predicted to have a detection rate of 95% (those that occur during maternal meiosis) for trisomy 21. If paternal samples are available, non-disjunctions that occur during paternal meiosis can also be detected. Thus, detection rates would be higher (about ˜99%) with a 0% false positive rate.

### Example 5

### Identification of Patients with Trisomy 21

A study was approved by the Institutional Review Board for Human Research at the Medical College of Wisconsin. 27 high risk pregnant patients who were scheduled to undergo amniocentesis or CVS or had already had either of these procedures were recruited as subjects at Froedtert Hospital. Informed consent was obtained from each participant before blood draws and buccal swabs were obtained.

Karyotype analysis confirmed 7 pregnancies carried a trisomy 21 fetus while 20 pregnancies carried a disomy 21 fetus. The gestational week and maternal age varied from 9-36.1 weeks and 22-43 years respectively. The gestational age ranged from first to third trimesters and the race and ethnicity of the subjects were (White (non-Hispanic), White (other), Asian, Other, and unknown).

The results from the study are shown in the table below:

TABLE 2
RESULTS OF STUDY CONFIRMED BY KARYOTYPE ANALYSIS

| | Fetal | Haplotype | | |
|---|---|---|---|---|
| | Chromosome 21 | ratio | | % of fetal DNA |
| Subject | status | (HR) | CV | (paternal contribution) |
| Subject 1 | Disomy | 1.21 | 0.39 | 7.4% |
| Subject 2 | Disomy | 1.19 | 0.06 | 10.8% |
| Subject 3 | Trisomy | 2.29 | 0.17 | 4.4% |
| Subject 4 | Trisomy | 2.55 | 0.22 | 1.3% |
| Subject 5 | Trisomy | 2.09 | 0.08 | 5.0% |
| Subject 6 | Trisomy | 2.33 | 0.26 | 3.3% |
| Subject 7 | Trisomy | 2.29 | 0.56 | 1.7% |
| Subject 8 | Trisomy | 0.05 | 0.05 | 7.1% |
| Subject 9 | Disomy | 0.95 | 0.16 | 5.5% |
| Subject 10 | Disomy | 1.04 | 0.05 | 19.7% |
| Subject 11 | Disomy | 1.00 | 0.05 | 8.0% |
| Subject 12 | Disomy | 0.99 | 0.31 | 6.8% |
| Subject 13 | Disomy | 0.90 | 0.26 | 7.3% |
| Subject 14 | Disomy | 1.04 | 0.05 | 24.0% |
| Subject 15 | Disomy | 1.15 | 0.12 | 4.0% |
| Subject 16 | Disomy | 1.06 | 0.11 | 2.9% |
| Subject 17 | Disomy | 0.92 | 0.26 | 4.0% |
| Subject 18 | Disomy | 1.35 | 0.02 | 11.7% |
| Subject 19 | Disomy | 0.95 | 0.13 | 9.2% |
| Subject 20 | Disomy | 1.29 | 0.23 | 3.7% |
| Subject 21 | Disomy | 1.46 | 0.07 | 7.0% |
| Subject 22 | Disomy | 1.21 | 0.14 | 6.4% |
| Subject 23 | Trisomy | 2.07 | 0.13 | 6.8% |
| Subject 24 | Disomy | 1.29 | 0.25 | 7.3% |
| Subject 25 | Disomy | 1.01 | 0.10 | 4.2% |
| Subject 26 | Disomy | 0.85 | 0.04 | 13.7% |
| Subject 27 | Disomy | 1.20 | 0.21 | 9.7% |

As is apparent from the above table, calculation of the haplotype ratio correctly identified each subject carrying a baby with trisomy and the haplotype ratio calculation was also able to correctly identify all normal pregnancies.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A method for determining whether a fetus has at least one genetic abnormality, said method comprising:
(a) detecting a paternally-inherited fetal allele in a sample comprising maternal and fetal nucleic acids, wherein said paternally-inherited fetal allele is not present in the maternal genome;
(b) detecting a first maternal allele and a second maternal allele in said sample;
(c) comparing said paternally-inherited fetal allele to said first and second maternal alleles, wherein said comparing identifies said at least one genetic abnormality.

2. A method according to claim 1, wherein said comparing comprises detecting the number of molecules of each of said paternally-inherited fetal allele, said first maternal allele and said second maternal allele.

3. A method according to claim 2, wherein said comparing comprises calculating an allelic dosage for said paternally-inherited fetal allele and for one or more maternally-inherited fetal alleles.

4. A method according to claim 3, wherein said paternally-inherited fetal allele, said first maternal allele and said second maternal allele are alleles of a tandem single nucleotide polymorphism (SNP).

5. A method according to claim 2, wherein said comparing comprises calculating a haplotype ratio.

6. A method according to claim 5, wherein said haplotype ratio is calculated according to the formula: HR=|P1−P2|/P3, wherein P1 is the number of molecules of said first maternal allele, P2 is the number of molecules of said second maternal allele, and P3 is the number molecules of said paternally-inherited fetal allele.

7. A method according to claim 1, wherein said comparing comprises determining the number of molecules of each of said paternally-inherited fetal allele, said first maternal allele and said second maternal allele, and wherein if the number of molecules of said first maternal allele and said second maternal allele is equal, said fetus has at least one genetic abnormality.

8. A method according to claim 1, wherein said comparing comprises determining the number of molecules present in said sample for each of said paternally-inherited fetal allele, said first maternal allele and said second maternal allele, and wherein if the number of molecules of the three alleles is in the relationship of peak, x and peak+2x, the fetus has at least one genetic abnormality.

9. A method according to claim 1, wherein said comparing comprises determining the number of molecules present in said sample for each of said paternally-inherited fetal allele, said first maternal allele and said second maternal allele, and wherein if the number of molecules of the three alleles is in the relationship of peak, x and peak+x, the fetus does not have a genetic abnormality.

10. A method according to claim 1, wherein said comparing comprises calculating an allelic ratio, and wherein said paternally-inherited fetal allele serves as an internal standard for said allelic ratio.

11. A method according to claim 1, wherein said paternally-inherited fetal allele and said first and second maternal allele each comprise a member selected from: a deletion, an insertion, a variant sequence, methylation, and any combination thereof.
